SEQUENCE LISTING (128 sequences)

SEQ ID NO: 1 - DNA, 3472 bp, Mouse
  gene (1)..(3472)
  misc_feature (1)..(2128)
  Intron (2129)..(2314)
  misc_feature (2315)..(3472)
  5′UTR (1)..(1516)
  CDS (1517)..(2128)
  CDS (2315)..(3106)
  3′UTR (3110)..(3472)
gtcgatccactagttctagagccccagtgaacatcatctcaaaatagctacttccctggctaagtcaggctctggggacctcagcctgtactcacagctatggagtaaaggtcatttttgatgaaaagtgatagaactgaggctatatcagcgagctctggtccctttgtttgtggtactgaagaaggaaaataggaccttttgcatgccagacaagctctgtaccaatgacccatgctccagccgttacctctagctctttgtgtccattctcaagatgaaaatcttcacatagctcttcatgtcctcactcaccatcctcctgtaccagttgttcagactacctacctacctacctacctagacctggccactggcaaggtcctgaaagcatttcaccttggtgtccacatcactgcctgccttctgagaaattactgagttgttccaatgcttccacactactcagaattcatgccactgcagcaggtgcagggcctctaatgtgccttttattaccctatctacatgacaaaatatcaggtagtgatgacattcctcatcctttagtctgaggagctctggggaattctgggatctctccatggcaagagtgttcagaaacaaagggaccactcaggagcagcgcagggttccataggacttgtatgtgtgagcagccccagaagccacacaggctctctctttcaatgctcaaggtgggctatcctgacatgacagggaaggtcgcagagtgctcagggtacgtgtagatggagtttctgacttgtcacgaacagctgccaagggttttcctctatgaagttacacttggcagtgcaaagggggcagctctcaggagaaagccatcagtttccagggcccagatcctttcctagtgaagaggctgaacagataaaaagagaacatgaatgagggctcctttggaaaactcccagttttcctctgcctcacctgctcaggcccttgtgttttccttgtctggcccggggaagggaaacagcagcccacacacaaattctgaggagcagggactggctatgtcctgtgtccaccagtttaccttttccttgtgggccaagagttctgctttgtcccagagtttggttatgagagagagagaaaagacacatcgtttcttcagcctcaatgacaaaattagggaaaggcgttggaactacctagctgagctcagcatctgggagggtaaacccacaagaagaggcaagaacatcgccaaatcccattccttttcccgaggtacacgatgaaagtaacctccctagaacttggtgctaaattaaatcttccttcatccaatctaaagccgctgccctctcccatcattactgcccaggggtctgtttctgtggatggggtgccagcgcggctctgaaacctgaaaagccctggaggacccctccttctgagacctagcccacaccagcagtcctctcccacacgggggtctcttcctttgagacagtgggagcagatggggggctctgcgctggggaggcctcagctggatcctgccgggaagaaaaaagggagccaccgcggaggggggcagccccgccccggtccgccagctctgctgcggattggcccgatgtctctatatctgggatgccttcctggcacgaagctaccacgtccccagtgtctccacccgacgtccatcagacctccgtacttggctttgcagtgcccgccactgtctcctgcgctcccgcgccgcgttccgcccaggccttgcccagctggaatgcagagatcgccgcccggctacggcgcacaggacgacccgccctcccgccgcgactgtgcatgggcccctggaatcggggccgctgctgaggcgcgcggcctccctgtcaccaacgtctcgcccacctcgcccgcctccccgtccagccttccgcggagcccaccgcgcagccccgaatcagggcgctatggctttggccgcggagagcgccaaactgccgacgagttgcgcattcggcggcccatgaacgccttcatggtgtgggcgaaggacgagcgcaagcgactggcgcaacaaaatccggatctgcacaacgcagtactgagcaagatgctgggtgagagcctcgaatgcttagaggggcggcgagggaggggactttgggagagggtcgggattcgggattttatggctggtcgctggggttctatgccatgtttcacgccgcgcgcccaaggcgcacgacggtttggctagtcgtgcgcccgacactggtccactaacaggctcccttcgcctacaggcaaagcgtggaaggagctgaacacggcggagaagcggcccttcgtggaagaggccgaacggctgcgtgtgcagcacttgcgcgaccatcccaactacaagtaccggcctcgccgcaaaaaacaggcgcgcaaggtccggaggctggagccgggcctcttgctcccgggcctcgtgcagccgtctgcgccgcccgaggccttcgctgcagcgtcagggtcagctcgctccttccgcgagctacccactctgggtgcggagttcgatggcttggggctacccacgcccgagcgctcgcctctggacggcctggagcctggcgaggcctccttcttcccaccgcctttggcgcccgaggactgcgctctgcgggctttccgggcaccctatgcccctgagctggcacgggacccgagcttctgctacggggcgcccctggctgaagcgctcaggacagcgccgcctgccgcgccactcgcaggtctctactatggcaccctgggcactccgggcccgtttcccaatcctctgtcaccaccacctgagtccccgtctcttgagggcacagagcaactggagcctaccgccgacctttgggccgatgtggacctcaccgaatttgaccagtatctcaattgcagccggactcgaccggatgccactacactcccctaccacgtggcactggccaaactaggtccgcgcgccatgtcctgtccagaagagagcagcctcatttctgcgctgtctgatgctagcagcgcggtctattacagtgcttgcatctcaggctagacactgtccttgccctccaccgcttctgcatgtggccaagtggcagagttgcctgctcccttcctttcgcatatgtatgttagggtatgcaacagcctttagagctggtggcctaaagatgccatttctgtcgcctcctcatttacacacctccttctgggggttacctgtgctttgggccttccctaggatcgtcaggccctggacgtgcaagctacctctgccaggattggtggtgaagaagctaaggcttttctgccatttatgttctagaatgaggctgttctgtttactttgccgggatatacatatatcatatataatacaatatatttaatttttaattaaacttttttctttaag

SEQ ID NO: 2 - protein, 468 aa, Mouse
  misc_feature (1)..(2128)
  misc_feature (2315)..(3472)
MGGSALGRPQLDPAGKKKGSHRGGGQPRPGPPALLRIGPMSLYLGCLPGTKLPRPQCLHPTSIRPPYLALQCPPLSPALPRRVPPRPCPAGMQRSPPGYGAQDDPPSRRDCAWAPGIGAAAEARGLPVTNVSPTSPASPSSLPRSPPRSPESGRYGFGRGERQTADELRIRRPMNAFMVWAKDERKRLAQQNPDLHNAVLSKMLGKAWKELNTAEKRPFVEEAERLRVQHLRDHPNYKYRPRRKKQARKVRRLEPGLLLPGLVQPSAPPEAFAAASGSARSFRELPTLGAEFDGLGLPTPERSPLDGLEPGEASFFPPPLAPEDCALRAFRAPYAPELARDPSFCYGAPLAEALRTAPPAAPLAGLYYGTLGTPGPFPNPLSPPPESPSLEGTEQLEPTADLWADVDLTEFDQYLNCSRTRPDATTLPYHVALAKLGPRAMSCPEESSLISALSDASSAVYYSACISG

SEQ ID NO: 3 - DNA, 3286 bp, Mouse
  5′UTR (1)..(1516)
  CDS (1517)..(2923)
  3′UTR (2924)..(3286)
gtcgatccactagttctagagccccagtgaacatcatctcaaaatagctacttccctggctaagtcaggctctggggacctcagcctgtactcacagctatggagtaaaggtcatttttgatgaaaagtgatagaactgaggctatatcagcgagctctggtccctttgtttgtggtactgaagaaggaaaataggaccttttgcatgccagacaagctctgtaccaatgacccatgctccagccgttacctctagctctttgtgtccattctcaagatgaaaatcttcacatagctcttcatgtcctcactcaccatcctcctgtaccagttgttcagactacctacctacctacctacctagacctggccactggcaaggtcctgaaagcatttcaccttggtgtccacatcactgcctgccttctgagaaattactgagttgttccaatgcttccacactactcagaattcatgccactgcagcaggtgcagggcctctaatgtgccttttattaccctatctacatgacaaaatatcaggtagtgatgacattcctcatcctttagtctgaggagctctggggaattctgggatctctccatggcaagagtgttcagaaacaaagggaccactcaggagcagcgcagggttccataggacttgtatgtgtgagcagccccagaagccacacaggctctctctttcaatgctcaaggtgggctatcctgacatgacagggaaggtcgcagagtgctcagggtacgtgtagatggagtttctgacttgtcacgaacagctgccaagggttttcctctatgaagttacacttggcagtgcaaagggggcagctctcaggagaaagccatcagtttccagggcccagatcctttcctagtgaagaggctgaacagataaaaagagaacatgaatgagggctcctttggaaaactcccagttttcctctgcctcacctgctcaggcccttgtgttttccttgtctggcccggggaagggaaacagcagcccacacacaaattctgaggagcagggactggctatgtcctgtgtccaccagtttaccttttccttgtgggccaagagttctgctttgtcccagagtttggttatgagagagagagaaaagacacatcgtttcttcagcctcaatgacaaaattagggaaaggcgttggaactacctagctgagctcagcatctgggagggtaaacccacaagaagaggcaagaacatcgccaaatcccattccttttcccgaggtacacgatgaaagtaacctccctagaacttggtgctaaattaaatcttccttcatccaatctaaagccgctgccctctcccatcattactgcccaggggtctgtttctgtggatggggtgccagcgcggctctgaaacctgaaaagccctggaggacccctccttctgagacctagcccacaccagcagtcctctcccacacgggggtctcttcctttgagacagtgggagcagatggggggctctgcgctggggaggcctcagctggatcctgccgggaagaaaaaagggagccaccgcggaggggggcagccccgccccggtccgccagctctgctgcggattggcccgatgtctctatatctgggatgccttcctggcacgaagctaccacgtccccagtgtctccacccgacgtccatcagacctccgtacttggctttgcagtgcccgccactgtctcctgcgctcccgcgccgcgttccgcccaggccttgcccagctggaatgcagagatcgccgcccggctacggcgcacaggacgacccgccctcccgccgcgactgtgcatgggcccctggaatcggggccgctgctgaggcgcgcggcctccctgtcaccaacgtctcgcccacctcgcccgcctccccgtccagccttccgcggagcccaccgcgcagccccgaatcagggcgctatggctttggccgcggagagcgccaaactgccgacgagttgcgcattcggcggcccatgaacgccttcatggtgtgggcgaaggacgagcgcaagcgactggcgcaacaaaatccggatctgcacaacgcagtactgagcaagatgctgggcaaagcgtggaaggagctgaacacggcggagaagcggcccttcgtggaagaggccgaacggctgcgtgtgcagcacttgcgcgaccatcccaactacaagtaccggcctcgccgcaaaaaacaggcgcgcaaggtccggaggctggagccgggcctcttgctcccgggcctcgtgcagccgtctgcgccgcccgaggccttcgctgcagcgtcagggtcagctcgctccttccgcgagctacccactctgggtgcggagttcgatggcttggggctacccacgcccgagcgctcgcctctggacggcctggagcctggcgaggcctccttcttcccaccgcctttggcgcccgaggactgcgctctgcgggctttccgggcaccctatgcccctgagctggcacgggacccgagcttctgctacggggcgcccctggctgaagcgctcaggacagcgccgcctgccgcgccactcgcaggtctctactatggcaccctgggcactccgggcccgtttcccaatcctctgtcaccaccacctgagtccccgtctcttgagggcacagagcaactggagcctaccgccgacctttgggccgatgtggacctcaccgaatttgaccagtatctcaattgcagccggactcgaccggatgccactacactcccctaccacgtggcactggccaaactaggtccgcgcgccatgtcctgtccagaagagagcagcctcatttctgcgctgtctgatgctagcagcgcggtctattacagtgcttgcatctcaggctagacactgtccttgccctccaccgcttctgcatgtggccaagtggcagagttgcctgctcccttcctttcgcatatgtatgttagggtatgcaacagcctttagagctggtggcctaaagatgccatttctgtcgcctcctcatttacacacctccttctgggggttacctgtgctttgggccttccctaggatcgtcaggccctggacgtgcaagctacctctgccaggattggtggtgaagaagctaaggcttttctgccatttatgttctagaatgaggctgttctgtttactttgccgggatatacatatatcatatataatacaatatatttaatttttaattaaacttttttctttaag

SEQ ID NO: 4 - protein, 468 aa, Mouse; identical to SEQ ID NO: 2

SEQ ID NO: 5 - DNA, 1407 bp, Mouse
atggggggctctgcgctggggaggcctcagctggatcctgccgggaagaaaaaagggagccaccgcggaggggggcagccccgccccggtccgccagctctgctgcggattggcccgatgtctctatatctgggatgccttcctggcacgaagctaccacgtccccagtgtctccacccgacgtccatcagacctccgtacttggctttgcagtgcccgccactgtctcctgcgctcccgcgccgcgttccgcccaggccttgcccagctggaatgcagagatcgccgcccggctacggcgcacaggacgacccgccctcccgccgcgactgtgcatgggcccctggaatcggggccgctgctgaggcgcgcggcctccctgtcaccaacgtctcgcccacctcgcccgcctccccgtccagccttccgcggagcccaccgcgcagccccgaatcagggcgctatggctttggccgcggagagcgccaaactgccgacgagttgcgcattcggcggcccatgaacgccttcatggtgtgggcgaaggacgagcgcaagcgactggcgcaacaaaatccggatctgcacaacgcagtactgagcaagatgctgggcaaagcgtggaaggagctgaacacggcggagaagcggcccttcgtggaagaggccgaacggctgcgtgtgcagcacttgcgcgaccatcccaactacaagtaccggcctcgccgcaaaaaacaggcgcgcaaggtccggaggctggagccgggcctcttgctcccgggcctcgtgcagccgtctgcgccgcccgaggccttcgctgcagcgtcagggtcagctcgctccttccgcgagctacccactctgggtgcggagttcgatggcttggggctacccacgcccgagcgctcgcctctggacggcctggagcctggcgaggcctccttcttcccaccgcctttggcgcccgaggactgcgctctgcgggctttccgggcaccctatgcccctgagctggcacgggacccgagcttctgctacggggcgcccctggctgaagcgctcaggacagcgccgcctgccgcgccactcgcaggtctctactatggcaccctgggcactccgggcccgtttcccaatcctctgtcaccaccacctgagtccccgtctcttgagggcacagagcaactggagcctaccgccgacctttgggccgatgtggacctcaccgaatttgaccagtatctcaattgcagccggactcgaccggatgccactacactcccctaccacgtggcactggccaaactaggtccgcgcgccatgtcctgtccagaagagagcagcctcatttctgcgctgtctgatgctagcagcgcggtctattacagtgcttgcatctcaggctag

SEQ ID NO: 6 - DNA, 273 bp, Mouse
  CDS (1)..(273): Novel murine Sox18 5′ coding sequence
atggggggctctgcgctggggaggcctcagctggatcctgccgggaagaaaaaagggagccaccgcggaggggggcagccccgccccggtccgccagctctgctgcggattggcccgatgtctctatatctgggatgccttcctggcacgaagctaccacgtccccagtgtctccacccgacgtccatcagacctccgtacttggctttgcagtgcccgccactgtctcctgcgctcccgcgccgcgttccgcccaggccttgcccagctgga

SEQ ID NO: 7 - protein, 91 aa, Mouse
MGGSALGRPQLDPAGKKKGSHRGGGQPRPGPPALLRIGPMSLYLGCLPGTKLPRPQCLHPTSIRPPYLALQCPPLSPALPRRVPPRPCPAG

SEQ ID NO: 8 - DNA, 237 bp, Mouse
  CDS (1)..(237): HMG box
ttgcgcattcggcggcccatgaacgccttcatggtgtgggcgaaggacgagcgcaagcgactggcgcaacaaaatccggatctgcacaacgcagtactgagcaagatgctgggcaaagcgtggaaggagctgaacacggcggagaagcggcccttcgtggaagaggccgaacggctgcgtgtgcagcacttgcgcgaccatcccaactacaagtaccggcctcgccgcaaaaaacag

SEQ ID NO: 9 - protein, 79 aa, Mouse
LRIRRPMNAFMVWAKDERKRLAQQNPDLHNAVLSKMLGKAWKELNTAEKRPFVEEAERLRVQHLRDHPNYKYRPRRKKQ

SEQ ID NO: 10 - DNA, 282 bp, Mouse
  CDS (1)..(282): Trans-activation domain
gtccggaggctggagccgggcctcttgctcccgggcctcgtgcagccgtctgcgccgcccgaggccttcgctgcagcgtcagggtcagctcgctccttccgcgagctacccactctgggtgcggagttcgatggcttggggctacccacgcccgagcgctcgcctctggacggcctggagcctggcgaggcctccttcttcccaccgcctttggcgcccgaggactgcgctctgcgggctttccgggcaccctatgcccctgagctggcacgggacccgagc

SEQ ID NO: 11 - protein, 94 aa, Mouse
VRRLEPGLLLPGLVQPSAPPEAFAAASGSARSFRELPTLGAEFDGLGLPTPERSPLDGLEPGEASFFPPPLAPEDCALRAFRAPYAPELARDPS

SEQ ID NO: 12 - DNA, 264 bp, Mouse
  CDS (1)..(264): Conserved C-terminal domain
ctgtcaccaccacctgagtccccgtctcttgagggcacagagcaactggagcctaccgccgacctttgggccgatgtggacctcaccgaatttgaccagtatctcaattgcagccggactcgaccggatgccactacactcccctaccacgtggcactggccaaactaggtccgcgcgccatgtcctgtccagaagagagcagcctcatttctgcgctgtctgatgctagcagcgcggtctattacagtgcttgcatctcaggc

SEQ ID NO: 13 - protein, 88 aa, Mouse
LSPPPESPSLEGTEQLEPTADLWADVDLTEFDQYLNCSRTRPDATTLPYHVALAKLGPRAMSCPEESSLISALSDASSAVYYSACISG

SEQ ID NO: 14 - DNA, 1421 bp, Human
  CDS (1)..(1023): Partial human Sox 18 coding sequence
  3′UTR (1024)..(1420)
gggcgggcgcccgcctcgccgcccagcccgcagcgcagtcccccgcgcagccccgagccggggcgctatggcctcagcccggccggccgcggggaacgccaggcggcagacgagtcgcgcatccggcggcccatgaacgccttcatggtgtgggcaaaggacgagcgcaagcggctggctcagcagaacccggacctgcacaacgcggtgctcagcaagatgctgggcaaagcgtggaaggagctgaacgcggcggagaagcggcccttcgtggaggaagccgaacggctgcgcgtgcagcacttgcgcgaccaccccaactacaagtaccggccgcgccgcaagaagcaggcgcgcaaggcccggcggctggagcccggcctcctgctcccgggattagcgcccccgcagccaccgcccgagcctttccccgcggcgtctggctcggctcgcgccttccgcgagctgcccccgctgggcgccgagttcgacggcctggggctgcccacgcccgagcgctcgcctctggacggcctggagcccggcgaggctgccttcttcccaccgcccgcggcgcccgaggactgcgcgctgcggcccttccgcgcgccctacgcgcccaccgagttgtcgcgggaccccggcggttgctacggggctcccctggcggaggcgctcaggaccgcgccccccgcggcgccgctcgctggcctgtactacggcaccctgggcacgcccggcccgtaccccggcccgctgtcgccgccgcccgaggccccgccgctggagagcgccgagccgctggggcccgccgccgatctgtgggccgacgtggacctcaccgagttcgaccagtacctcaactgcagccggactcggcccgacgcccccgggctcccgtaccacgtggcactggccaaactgggcccgcgcgccatgtcctgcccagaggagagcagcctgatctccgcgctgtcggacgccagcagcgcggtctattacagcgcgtgcatctccggctaggccgccggcgccgcccgggtccctgcagcgcttcctcccgcagcccccgcgaccgatccgaccgcgtcgctgccgctctgctctctcatacgcgtgtatgtttggttccatgtcacagccccctaggagccagtgatgctcggccttgcgcccgttccacctcccaggccacccttcctgggcttctgggccacctgccctcggggggcccctgcgagggtgcctggagttcccacgtgtcccggggcttttccaggaagcccgagcccaggacctgttggcagagttgccagggttacatttttgaagcacctgctccttttcttgcagtgtattttctacaaccagattgtattaatattttttactttgcccttttaaaaaatatacctaatccc

SEQ ID NO: 15 - protein, 340 aa, Human
GRAPASPPSPQRSPPRSPEPGRYGLSPAGRGERQAADESRIRRPMNAFMVWAKDERKRLAQQNPDLHNAVLSKMLGKAWKELNAAEKRPFVEEAERLRVQHLRDHPNYKYRPRRKKQARKARRLEPGLLLPGLAPPQPPPEPFPAASGSARAFRELPPLGAEFDGLGLPTPERSPLDGLEPGEAAFFPPPAAPEDCALRPFRAPYAPTELSRDPGGCYGAPLAEALRTAPPAAPLAGLYYGTLGTPGPYPGPLSPPPEAPPLESAEPLGPAADLWADVDLTEFDQYLNCSRTRPDAPGLPYHVALAKLGPRAMSCPEESSLISALSDASSAVYYSACISG

SEQ ID NO: 16 - DNA, 1023 bp, Human
gggcgggcgcccgcctcgccgcccagcccgcagcgcagtcccccgcgcagccccgagccggggcgctatggcctcagcccggccggccgcggggaacgccaggcggcagacgagtcgcgcatccggcggcccatgaacgccttcatggtgtgggcaaaggacgagcgcaagcggctggctcagcagaacccggacctgcacaacgcggtgctcagcaagatgctgggcaaagcgtggaaggagctgaacgcggcggagaagcggcccttcgtggaggaagccgaacggctgcgcgtgcagcacttgcgcgaccaccccaactacaagtaccggccgcgccgcaagaagcaggcgcgcaaggcccggcggctggagcccggcctcctgctcccgggattagcgcccccgcagccaccgcccgagcctttccccgcggcgtctggctcggctcgcgccttccgcgagctgcccccgctgggcgccgagttcgacggcctggggctgcccacgcccgagcgctcgcctctggacggcctggagcccggcgaggctgccttcttcccaccgcccgcggcgcccgaggactgcgcgctgcggcccttccgcgcgccctacgcgcccaccgagttgtcgcgggaccccggcggttgctacggggctcccctggcggaggcgctcaggaccgcgccccccgcggcgccgctcgctggcctgtactacggcaccctgggcacgcccggcccgtaccccggcccgctgtcgccgccgcccgaggccccgccgctggagagcgccgagccgctggggcccgccgccgatctgtgggccgacgtggacctcaccgagttcgaccagtacctcaactgcagccggactcggcccgacgcccccgggctcccgtaccacgtggcactggccaaactgggcccgcgcgccatgtcctgcccagaggagagcagcctgatctccgcgctgtcggacgccagcagcgcggtctattacagcgcgtgcatctccggctag

SEQ ID NO: 17 - DNA, 1919 bp, Human
  gene (1)..(1919): Exon 1
  misc_feature (1)..(482)
  Intron (483)..(678): Intron 1
  misc_feature (679)..(1919): Exon 2
  CDS (126)..(482)
  CDS (679)..(1473)
ccaccgccgtccccaccgccatccgccctcccggcctggcctgcccttgcgcccggctccccagtgcccgccgcccgcccgccgcgctcccgcgctccgttccgcccaggccgcgcccagctggaatgcagagatcgccgcccggctacggcgcacaggacgacccgcccgcccgccgcgactgtgcatgggccccgggacacggggccgccgctgacacgcgcggcctcgccgccggccccgccgccctcgccgcgcccgccgcgcccgcctcgccgcccagcccgcagcgcagtcccccgcgcagccccgagccggggcgctatggcctcagcccggccggccgcggggaacgccaggcggcagacgagtcgcgcatccggcggcccatgaacgccttcatggtgtgggcaaaggacgagcgcaagcggctggctcagcagaacccggacctgcacaacgcggtgctcagcaagatgctgggtgagcggcgggagggcggcagagaagggggagagggcggggggggctcgggccggggtggggcggggggggcaggggtcccgggccgcggggtgcgggggctgcgccaaaccctcgcggggcgtcccgggcgcacggagggctcggcgcccacccgcccgacggcgttccactcactggcgcccacggcccgcaggcaaagcgtggaaggagctgaacgcggcggagaagcggcccttcgtggaggaagccgaacggctgcgcgtgcagcacttgcgcgaccaccccaactacaagtaccggccgcgccgcaagaagcaggcgcgcaaggcccggcggctggagcccggcctcctgctcccgggattagcgcccccgcagccaccgcccgagcctttccccgcggcgtctggctcggctcgcgccttccgcgagctgcccccgctgggcgccgagttcgacggcctggggctgcccacgcccgagcgctcgcctctggacggcctggagcccggcgaggctgccttcttcccaccgcccgcggcgcccgaggactgcgcgctgcggcccttccgcgcgccctacgcgcccaccgagttgtcgcgggaccccggcggttgctacggggctcccctggcggaggcgctcaggaccgcgccccccgcggcgccgctcgctggcctgtactacggcaccctgggcacgcccggcccgtaccccggcccgctgtcgccgccgcccgaggccccgccgctggagagcgccgagccgctggggcccgccgccgatctgtgggccgacgtggacctcaccgagttcgaccagtacctcaactgcagccggactcggcccgacgcccccgggctcccgtaccacgtggcactggccaaactgggcccgcgcgccatgtcctgcccagaggagagcagcctgatctccgcgctgtcggacgccagcagcgcggtctattacagcgcgtgcatctccggctaggccgccggcgccgcccgggtccctgcagcgcttcctcccgcagcccccgcgaccgatccgaccgcgtcgctgccgctctgctctctcatacgcgtgtatgtttggttccatgtcacagccccctaggagccagtgatgctcggccttgcgcccgttccacctcccaggccacccttcctgggcttctgggccacctgccctcggggggcccctgcgagggtgcctggagttcccacgtgtcccggggcttttccaggaagcccgagcccaggacctgttggcagagttgccagggttacatttttgaagcacctgctccttttcttgcagtgtattttctacaaccagattgtattaatattttttactttgcccttttaaaaaatatacctaatacaatatatttaatttttaattaaactcttaaacttttcttccaagaa

SEQ ID NO: 18 - protein, 384 aa, Human [sequence omitted]

SEQ ID NO: 19 - DNA, 1730 bp, Human
  CDS (123)..(1277)
  5′UTR (1)..(122)
  3′UTR (1278)..(1730)
ccacgcgtccgccgccatccgccctcccggcctggcctgcccttgcgcccggctccccagtgcccgccgctcgcctcgccgcgctcccgcgctccgttccgcccaggccgcgcccagctggaatgcagagatcgccgcccggctacggcgcacaggacgacccgcccgcccgccgcgactgtgcatgggccccgggacacggggccgccgctgacacgcgcggcctcgccgccggccccgccgccctcgccgcgcccgccgcgcccgcctcgccgcccagcccgcagcgcagtcccccgcgcagccccgagccggggcgctatggcctcagcccggccggccgcggggaacgccaggcggcagacgagtcgcgcatccggcggcccatgaacgccttcatggtgtgggcaaaggacgagcgcaagcggctggctcagcagaacccggacctgcacaacgcggtgctcagcaagatgctgggcaaagcgtggaaggagctgaacgcggcggagaagcggcccttcgtggaggaagccgaacggctgcgcgtgcagcacttgcgcgaccaccccaactacaagtaccggccgcgccgcaagaagcaggcgcgcaaggcccggcggctggagcccggcctcctgctcccgggattagcgcccccgcagccaccgcccgagcctttccccgcggcgtctggctcggctcgcgccttccgcgagctgcccccgctgggcgccgagttcgacggcctggggctgcccacgcccgagcgctcgcctctggacggcctggagcccggcgaggctgccttcttcccaccgcccgcggcgcccgaggactgcgcgctgcggcccttccgcgcgccctacgcgcccaccgagttgtcgcgggaccccggcggttgctacggggctcccctggcggaggcgctcaggaccgcgccccccgcggcgccgctcgctggcctgtactacggcaccctgggcacgcccggcccgtaccccggcccgctgtcgccgccgcccgaggccccgccgctggagagcgccgagccgctggggcccgccgccgatctgtgggccgacgtggacctcaccgagttcgaccagtacctcaactgcagccggactcggcccgacgcccccgggctcccgtaccacgtggcactggccaaactgggcccgcgcgccatgtcctgcccagaggagagcagcctgatctccgcgctgtcggacgccagcagcgcggtctattacagcgcgtgcatctccggctaggccgccggcgccgcccgggtccctgcagcgcttcctcccgcagcccccgcgaccgatccgaccgcgtcgctgccgctctgctctctcatacgcgtgtatgtttggttccatgtcacagccccctaggagccagtgatgctcggccttgcgcccgttccacctcccaggccacccttcctgggcttctgggccacctgccctcggggggcccctgcgagggtgcctggagttcccacgtgtcccggggcttttccaggaagcccgagcccaggacctgttggcagagttgccagggttacatttttgaagcacctgctccttttcttgcagtgtattttctacaaccagattgtattaatattttttactttgcccttttaaaaaatatacctaatacaatatatttaatttttaattaaactcttaaacttttcttccaaaaaaaaaaaaaaa

SEQ ID NO: 20 - protein, 384 aa, Human [sequence omitted]

SEQ ID NO: 21 - DNA, 1155 bp, Human
atgcagagatcgccgcccggctacggcgcacaggacgacccgcccgcccgccgcgactgtgcatgggccccgggacacggggccgccgctgacacgcgcggcctcgccgccggccccgccgccctcgccgcgcccgccgcgcccgcctcgccgcccagcccgcagcgcagtcccccgcgcagccccgagccggggcgctatggcctcagcccggccggccgcggggaacgccaggcggcagacgagtcgcgcatccggcggcccatgaacgccttcatggtgtgggcaaaggacgagcgcaagcggctggctcagcagaacccggacctgcacaacgcggtgctcagcaagatgctgggcaaagcgtggaaggagctgaacgcggcggagaagcggcccttcgtggaggaagccgaacggctgcgcgtgcagcacttgcgcgaccaccccaactacaagtaccggccgcgccgcaagaagcaggcgcgcaaggcccggcggctggagcccggcctcctgctcccgggattagcgcccccgcagccaccgcccgagcctttccccgcggcgtctggctcggctcgcgccttccgcgagctgcccccgctgggcgccgagttcgacggcctggggctgcccacgcccgagcgctcgcctctggacggcctggagcccggcgaggctgccttcttcccaccgcccgcggcgcccgaggactgcgcgctgcggcccttccgcgcgccctacgcgcccaccgagttgtcgcgggaccccggcggttgctacggggctcccctggcggaggcgctcaggaccgcgccccccgcggcgccgctcgctggcctgtactacggcaccctgggcacgcccggcccgtaccccggcccgctgtcgccgccgcccgaggccccgccgctggagagcgccgagccgctggggcccgccgccgatctgtgggccgacgtggacctcaccgagttcgaccagtacctcaactgcagccggactcggcccgacgcccccgggctcccgtaccacgtggcactggccaaactgggcccgcgcgccatgtcctgcccagaggagagcagcctgatctccgcgctgtcggacgccagcagcgcggtctattacagcgcgtgcatctccggctag

SEQ ID NO: 22 - DNA, 237 bp, Human
  CDS (1)..(237): HMG box
tcgcgcatccggcggcccatgaacgccttcatggtgtgggcaaaggacgagcgcaagcggctggctcagcagaacccggacctgcacaacgcggtgctcagcaagatgctgggcaaagcgtggaaggagctgaacgcggcggagaagcggcccttcgtggaggaagccgaacggctgcgcgtgcagcacttgcgcgaccaccccaactacaagtaccggccgcgccgcaagaagcag

SEQ ID NO: 23 - protein, 79 aa, Human
SRIRRPMNAFMVWAKDERKRLAQQNPDLHNAVLSKMLGKAWKELNAAEKRPFVEEAERLRVQHLRDHPNYKYRPRRKKQ

SEQ ID NO: 24 - DNA, 282 bp, Human
  CDS (1)..(282): Transactivation domain
gcccggcggctggagcccggcctcctgctcccgggattagcgcccccgcagccaccgcccgagcctttccccgcggcgtctggctcggctcgcgccttccgcgagctgcccccgctgggcgccgagttcgacggcctggggctgcccacgcccgagcgctcgcctctggacggcctggagcccggcgaggctgccttcttcccaccgcccgcggcgcccgaggactgcgcgctgcggcccttccgcgcgccctacgcgcccaccgagttgtcgcgggacccc

SEQ ID NO: 25 - protein, 94 aa, Human
ARRLEPGLLLPGLAPPQPPPEPFPAASGSARAFRELPPLGAEFDGLGLPTPERSPLDGLEPGEAAFFPPPAAPEDCALRPFRAPYAPTELSRDP

SEQ ID NO: 26 - DNA, 264 bp, Human
  CDS (1)..(264): Conserved C-terminal domain
ctgtcgccgccgcccgaggccccgccgctggagagcgccgagccgctggggcccgccgccgatctgtgggccgacgtggacctcaccgagttcgaccagtacctcaactgcagccggactcggcccgacgcccccgggctcccgtaccacgtggcactggccaaactgggcccgcgcgccatgtcctgcccagaggagagcagcctgatctccgcgctgtcggacgccagcagcgcggtctattacagcgcgtgcatctccggc

SEQ ID NO: 27 - protein, 88 aa, Human
LSPPPEAPPLESAEPLGPAADLWADVDLTEFDQYLNCSRTRPDAPGLPYHVALAKLGPRAMSCPEESSLISALSDASSAVYYSACISG

SEQ ID NO: 28 - protein, 9 aa, Human
SDASSAVYY

SEQ ID NO: 29 - protein, 6 aa, Human
LSPPPE

SEQ ID NO: 30 - protein, 88 aa, Artificial Sequence
  Conserved C-terminal domain consensus
  VARIANT (7)..(7): Xaa is any amino acid, preferably Pro, Ala, Gly, Ser or Thr, more preferably Ala or Ser
  VARIANT (8)..(8): Xaa is any amino acid, preferably Pro, Ala, Gly, Ser or Thr, more preferably Ala
  VARIANT (9)..(9): Xaa is any amino acid, preferably Pro, Ala, Gly, Ser, Thr, His,Lys or Arg
  VARIANT (10)..(10): Xaa is any amino acid, preferably Leu, Ile, Val or Met, more preferably Leu or Met
  VARIANT (12)..(12): Xaa is any amino acid, preferably Pro, Ala, Gly, Ser or Thr, more preferably Gly or Ser
  VARIANT (13)..(13): Xaa is any amino acid, preferably Pro, Ala, Gly, Ser, Thr, Val,Leu or Ile, more preferably Ala
  VARIANT (14)..(14): Xaa is any amino acid, preferably Gln, Asn, Glu or Asp, more preferably Glu or Asp
  VARIANT (15)..(16): Xaa is any amino acid, preferably Gln, Asn, Glu, Asp, Pro, Ala, Gly, Ser, Thr, His, Lys or Arg
  VARIANT (17)..(17): Xaa is any amino acid, preferably Gln, Asn, Glu, Asp, Pro, Ala, Gly, Ser or Thr, more preferably Glu
  VARIANT (18)..(18): Xaa is any amino acid, preferably Gln, Asn, Glu, Asp, Pro, Ala, Gly, Ser or Thr
  VARIANT (19)..(19): Xaa is any amino acid, preferably Pro, Ala, Gly, Ser or Thr, more preferably Thr or Ala
  VARIANT (20)..(20): Xaa is any amino acid, preferably Pro, Ala, Gly, Ser or Thr, more preferably  Ala
  VARIANT (24)..(24): Xaa is any amino acid, preferably Pro, Ala, Gly, Ser or Thr, more preferably Thr or Ala
  VARIANT (28)..(28): Xaa is any amino acid, preferably Leu, Val, Ile, His, Lys or Arg, more preferably Leu
  VARIANT (29)..(29): Xaa is any amino acid, preferably Pro, Ala, Gly, Ser, Thr, Gln, Asn, Glu or Asp
  VARIANT (37)..(37): Xaa is any amino acid, preferably Cys, Ser, Met, Ile, Leu or Val
  VARIANT (43)..(43): Xaa is any amino acid, preferably Gln, Asn, Glu or Asp, more preferably Asp or Glu
  VARIANT (45)..(45): Xaa is any amino acid, preferably Pro, Ala, Gly, Ser or Thr, more preferably Pro
  VARIANT (46)..(46): Xaa is any amino acid, preferably Pro, Ala, Gly, Ser or Thr, more preferably Glu
  VARIANT (52)..(52): Xaa is any amino acid, preferably Pro, Ala, Gly, Ser or Thr, more preferably Ala
  VARIANT (54)..(54): Xaa is any amino acid, preferably Pro, Ala, Gly, Ser or Thr, more preferably Ala
  VARIANT (56)..(56): Xaa is any amino acid, preferably Leu, Ile or Val, more preferably Leu or Val
  VARIANT (57)..(57): Xaa is any amino acid, preferably Pro, Ala, Gly, Ser or Thr, more preferably Gly
  VARIANT (60)..(60): Xaa is any amino acid, preferably Pro, Ala, Gly, Ser or Thr, more preferably Ala
  VARIANT (61)..(61): Xaa is any amino acid, preferably Leu, Ile, Val or Met, more preferably Ile or Met
  VARIANT (64)..(64): Xaa is any amino acid, preferably Pro, Ala, Gly, Ser or Thr, more preferably Pro
  VARIANT (84)..(84): Xaa is any amino acid, preferably Pro, Ala, Gly, Ser or Thr, more preferably Pro
  VARIANT (87)..(87): Xaa is any amino acid, preferably Pro, Ala, Gly, Ser or Thr, more preferably Ser
LSPPPEXXXXEXXXXXXXXXDLWXDVDXXEFDQYLNXSRTRPXAXXLPYHVXLXKXXPRXXSCXEESSLISALSDASSAVYYSXCIXG

SEQ ID NO: 31 - DNA, 1593 bp, Chicken
  CDS (226)..(1479)
tttttttgggggggggaaaacggggaaaaaaaaaaaaaaagaaaaagagcaaaggacagaaaccaaaacaacccaaggcgaccccaaatccccccgaaaccacccgaacgcgggcgaagaagcgagcggaggtgctcggcggctgcaggaggcggcgggacgcgcgggactcggttctctctccggcacccgctcgggtcggacacaaagtgctgtccagctggaatgaatatatctgagtcaaactactgccgagaggagatatcgcaaccccggggcgactgttcatgggtcaccggcgccgtgccggccgctgagcccgggctcgccttccctcggcccccgggagccgcctccccctccagccgcacgcccagccccgagcccggcttcgccttcggccccgcggcccccggggcggcccccggagcggcccccagccgcacgcccagccccgagccgggctatggatacagccccccggcgggccgagccgaagggaaggccggggaggattcccgcatccgccgccccatgaacgccttcatggtttgggcgaaggatgagcgcaagcggctggcgcagcaaaaccccgacctgcacaacgccgtgctcagcaagatgctgggccaatcgtggaaagccttgagcgccagcgacaagcgtccctttgtggaagaggccgagcggctgcgaatccagcacctccaggatcaccccaactacaagtaccgcccgaggaggaagaagcaagccaagaaaatcaagaggatggaacccaatatcctcctgcataacctttcccagccttgcagtgacaacttcagcatgagtcaccacagcggcagccagccgggccacccccagcctcccccactcaaccacttcagagaactccactccatggggtcggatattgaaaactatggcttgccaactcccgagatgtctcccttggatgtcttggaacagaccgagccggcgtttttccccccgcacatgcaggaggactgcagcatgatgcccttccgcgggtaccaccaccaccacccccagatggagtttccccaggagaagtgcctgggccgggacgtggccgtgccctacgcgcagcccccggcacacttggccgatgccatgaggactccccacccctccggcctctactacaaccagatgtgctcggggactcagagcgggctttccgcccacctgggccagctctcccccccacccgaagcccaccacatggagagcgtggatcacttgaaccaaaccgacctttggacggacgttgaccgcaatgagtttgaccagtatttgaacatgagcaggactcgtcccgaagcctcgggactgccttatcatgtctccctgtccaaagtgactcctaggagcatctcctgcgaggagagcagcttgatatccgccctgtccgacgccagcagcgccgtctactacagcccatgcatcaccggctaggctcgcgcgtgcgcccaacgcaacaagccaacgagcagccatcctcctccaaatgcgagctccgtaatatgtagagtatctcattaaatgcatcgctttctttcttaaaaa

SEQ ID NO: 32 - protein, 418 aa, Chicken
MNISESNYCREEISQPRGDCSWVTGAVPAAEPGLAFPRPPGAASPSSRTPSPEPGFAFGPAAPGAAPGAAPSRTPSPEPGYGYSPPAGRAEGKAGEDSRIRRPMNAFMVWAKDERKRLAQQNPDLHNAVLSKMLGQSWKALSASDKRPFVEEAERLRIQHLQDHPNYKYRPRRKKQAKKIKRMEPNILLHNLSQPCSDNFSMSHHSGSQPGHPQPPPLNHFRELHSMGSDIENYGLPTPEMSPLDVLEQTEPAFFPPHMQEDCSMMPFRGYHHHHPQMEFPQEKCLGRDVAVPYAQPPAHLADAMRTPHPSGLYYNQMCSGTQSGLSAHLGQLSPPPEAHHMESVDHLNQTDLWTDVDRNEFDQYLNMSRTRPEASGLPYHVSLSKVTPRSISCEESSLISALSDASSAVYYSPCITG

SEQ ID NO: 33 - DNA, 3266 bp, Mouse
  CDS (51)..(1193)
  misc_feature (180)..(392): HMG; Region: high mobility group
  misc_feature (183)..(389): HMG box
  polyA_signal (3246)..(3251)
caggtcagcgccggccccacgaggcgaagccaagtgacccgcgttcggccatggcctcgctgctgggcgcctatccgtggaccgagggactggagtgtcccgccctggaagccgagctgtcggatgggctgtcgccgcccgccgtcccccgaccttcaggggacaagagttcggaaagccggatccggcggcccatgaatgccttcatggtgtgggccaaggatgagaggaaacgtctggcagtgcagaacccggacctgcacaacgcggagctcagcaagatgctgggaaagtcatggaaggcgctgacactgtcccagaagagaccctatgtggatgaggcagagcggctgcgcctgcagcacatgcaggattaccccaactacaagtaccggccccgcaggaagaaacaaggcaagcgcctctgcaagcgcgtggaccctggcttcctcctcagctccctctctcgtgaccagaacacgctgcctgagaaaaacggcattggcaggggggagaaggaggacaggggtgagtactccccaggggccaccttgcctggactgcacagctgctaccgcgaaggtgcagctgctgcccctggcagtgtggacacgtatccctacgggctgcccacacctccggagatgtcgcccctggatgcgctggagccagagcagaccttcttctcgtcctcatgtcaggaggagcatggtcacccccatcacctcccccatctaccagggcccccttactcaccggagttcacacctagtcccctccactgcagccaccctctaggttctttagcccttggccaatccccaggggtttctatgatgtcctctgtttctggatgtcccccatctccagcctattactcccatgccacctaccaccctctccaccccaacctccaggcccacctgggccagctgtccccacctccggagcaccctggctttgacaccttggatcagctaagccaggtggaacttctgggagacatggatcgcaatgaatttgatcagtatttgaacactcccggccaccctgactctgctgcaggggttggaaccctcactgggcatgtcccgctctcccaggggactcccacaggccctacagagaccagcctcatctcagtcctggctgatgccacggccacgtattacaacagctacagtgtgtcatagagctggaggaatggagcctggcccagccctgccatcccctcctccctatgaagcactgagctgagcagaaactgtggcgatgccactccttggtggcaagaggtcaaaactgctgttccagagccttccggtcaaaagccaggatccagcgctccttctgtccctttaaatgtgttttgtcggcttcatcaaggaagcacagccttgggagctgtttgcagtaggatttcctagccgctctacaccatcagtgtagctgatgggaagaagcccagtgcattggagctcctgcttttggtgtagctacttctgatctgtggtgctcccagtaagatggaccctgggcctggggtccatctcagttcagtctctaactggcaaaaccccactctggcttgaccagccctgactattctctcctgtacagtgagggttggcttcatagccaagccctcgctctatggatctccaagttacagccctctcaaggccaaggaaagtatgaatgaatgactgcccaaggacccaggttttctgctttcctataatactgtccacagccaccctagaattccaaagaaactccttcagtcgagaaaaatattcacctttggtctggcaagatggtgtgggatgtctcagtgggtaagactgcctgctgccaagtctgatgacctacgttctatatccggagcctacatggtggaagaagagaactgactcctgcaagttgtattctgacttccaaatgcacgtgtgcccctgcattatatgtgtgcttgagcatgctcacatgcacacacacacacacacacacactaaatgtaatttttttaaaggaaaagatctaccctggttcggaaaattaatgagtatgttcaactgcatcactgaaagcaaaatcagattagaacttgttttgttttgttttgttttgttttgttttgttttgttttgttttgttctgagacagggtttctctgtgtaaccctggctgccctggaactcactgtgtagaccaggctggcctcaacctcaaaagtaagcctgcctctgcctcctgggtactgggattaaaggtgagtggcaccacacccagcctggatttttttttaatatatactttagatacattatttttgttacatacatctcaggcgactacagtgtgagaaatgaccttatgcaggcatgcctattcccaacactgtgccccaggtgtgtaccctgatttagattcttctcaactgtgacaatgtctcatcccctcatcacagaatgaaccagagagctagaaacctaaaacgtctgtccacaaaagtgttggggtgtgctgatttctagagcagagctgtagcatactcttatctacagatgcaaaagatgactctctgcccaatgtctgctgccccttgctcccaggagggcttttctctcttctctgttactctccctccaaactggcccctaattcaaagggcccctcgcttggaatcccaggcaccattcccctgggcgtgaagggaacttggttcttgtttctcaagtggatacatcagaacaatcacctatcccacagtcctttggctgtcccaaacaaacaccctgtgacacccaaagatgtttctgagctttggtcgcaccccagacatttccattctctaaacctcctttgaaggaacataccaaacactgagctaacagactcttgtctgcccttggacacgtgtatattattgcctttatgaaatgtgtttttatattctgctattatgtccaccttatattttgtattgttgacagatccccaagaagtcttattataatatcacttataagatatacttaatctttatgctattactgtatataccatttggtaacaaatagtgaattgttgatgatgtaattgactggtgcccagtcccggatggatattaataatctcgtgaaacactatcacaggcattaagctactctgttccattttttcaagaaaactaaatatgcatggaacagttatgaaattgatttttcaaaatgtttaatttaaataaatttttttgtaccttc

SEQ ID NO: 34 - protein, 380 aa, Mouse
  misc_feature (180)..(392): HMG; Region: high mobility group
  misc_feature (183)..(389): HMG box
MASLLGAYPWTEGLECPALEAELSDGLSPPAVPRPSGDKSSESRIRRPMNAFMVWAKDERKRLAVQNPDLHNAELSKMLGKSWKALTLSQKRPYVDEAERLRLQHMQDYPNYKYRPRRKKQGKRLCKRVDPGFLLSSLSRDQNTLPEKNGIGRGEKEDRGEYSPGATLPGLHSCYREGAAAAPGSVDTYPYGLPTPPEMSPLDALEPEQTFFSSSCQEEHGHPHHLPHLPGPPYSPEFTPSPLHCSHPLGSLALGQSPGVSMMSSVSGCPPSPAYYSHATYHPLHPNLQAHLGQLSPPPEHPGFDTLDQLSQVELLGDMDRNEFDQYLNTPGHPDSAAGVGTLTGHVPLSQGTPTGPTETSLISVLADATATYYNSYSVS

SEQ ID NO: 35 - DNA, 1512 bp, Mouse
  CDS (250)..(1509)
  misc_feature (448)..(654): HMG; Region: high mobility group
  misc_feature (451)..(654): HMG box
ctgaagtgcggttggccccaacactcctcccaaagtatctatcaagagaatggtcagcagaagttagatctagtgagcagcacctccagacatctgaatttcagccttcctatttccccaagaggtcttggcgccagcgcccggctccagccagttttccccaaggctagcttccgatccctgcctcagggtcgggggaagcggcgtgtcccgtggccatagcagagctcggggtcggtctggagagccatgagcagcccggatgcgggatacgccagtgacgaccagagccagccccggagcgcgcagcccgcggtgatggcagggttgggcccctgtccctgggccgagtccctgagccccctcggggatgtaaaggtgaaaggcgaggtggtggcgagtagcggggcgccagccgggacgtcgggccgagccaaagcggagtctcgcatccggcggccgatgaacgcctttatggtgtgggccaaagacgaacgcaagcggttggcacagcagaacccagatctgcacaacgcagagctaagcaagatgctaggcaagtcttggaaggcgttgaccttggcagagaagcggcccttcgtggaagaggccgagcggctgcgcgtgcagcatatgcaggaccaccccaactacaagtaccggccgcggcggcgcaagcaggtgaagcgcatgaagcgggtggagggaggcttcctgcacgctctcgtcgagccccaggccggcgcgcttggtcccgagggcggccgcgtggccatggatggcctgggtctgcctttcccggagccgggctatccggccggtcctccgctgatgtctccgcacatgggcccccactatcgggactgccagggactgggcgctcccgcgctcgacggctaccctctgcccactccggacacatccccgctggatggcgtggagcaggacccggctttctttgcagccccgctgccaggggactgcccggcggccggcacctacacttacgctccagtctcggactatgcagtgtccgtagagccgcccgctggccccatgcgagtggggccggacccctcgggccctgcgatgccggggatcctggcgccccccagcgctctgcacctgtactacggcgcgatgggctcgcccgccgcaagtgcggggcgcggtttccacgcgcaaccccagcagccgctgcaaccgcaggcaccgccgccgccaccgcagcagcagcacccagcgcacggccccgggcaaccttcgccccctcccgaggctctgccctgccgggatggcacggaatccaaccagcccactgagctcctaggggaggtggaccgcacggaattcgaacagtatctgccctttgtgtataagcccgagatgggtcttccctaccagggacacgactgcggagtgaacctctcagacagccacggagccatttcctccgtggtgtccgacgctagctcagcggtctactattgcaactaccccgacatttgacgg

SEQ ID NO: 36 - protein, 419 aa, Mouse
  misc_feature (448)..(654): HMG; Region: high mobility group
  misc_feature (451)..(654): HMG box
MSSPDAGYASDDQSQPRSAQPAVMAGLGPCPWAESLSPLGDVKVKGEVVASSGAPAGTSGRAKAESRIRRPMNAFMVWAKDERKRLAQQNPDLHNAELSKMLGKSWKALTLAEKRPFVEEAERLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAPPPPPQQQHPAHGPGQPSPPPEALPCRDGTESNQPTELLGEVDRTEFEQYLPFVYKPEMGLPYQGHDCGVNLSDSHGAISSVVSDASSAVYYCNYPDI

SEQ ID NO: 37 - protein, 26 aa, Mouse
CALRAFRAPYAPELARDPSFCYGAPL

SEQ ID NO: 38 - DNA, 1701 bp, Mouse
gtcgatccactagttctagagccccagtgaacatcatctcaaaatagctacttccctggctaagtcaggctctggggacctcagcctgtactcacagctatggagtaaaggtcatttttgatgaaaagtgatagaactgaggctatatcagcgagctctggtccctttgtttgtggtactgaagaaggaaaataggaccttttgcatgccagacaagctctgtaccaatgacccatgctccagccgttacctctagctctttgtgtccattctcaagatgaaaatcttcacatagctcttcatgtcctcactcaccatcctcctgtaccagttgttcagactacctacctacctacctacctagacctggccactggcaaggtcctgaaagcatttcaccttggtgtccacatcactgcctgccttctgagaaattactgagttgttccaatgcttccacactactcagaattcatgccactgcagcaggtgcagggcctctaatgtgccttttattaccctatctacatgacaaaatatcaggtagtgatgacattcctcatcctttagtctgaggagctctggggaattctgggatctctccatggcaagagtgttcagaaacaaagggaccactcaggagcagcgcagggttccataggacttgtatgtgtgagcagccccagaagccacacaggctctctctttcaatgctcaaggtgggctatcctgacatgacagggaaggtcgcagagtgctcagggtacgtgtagatggagtttctgacttgtcacgaacagctgccaagggttttcctctatgaagttacacttggcagtgcaaagggggcagctctcaggagaaagccatcagtttccagggcccagatcctttcctagtgaagaggctgaacagataaaaagagaacatgaatgagggctcctttggaaaactcccagttttcctctgcctcacctgctcaggcccttgtgttttccttgtctggcccggggaagggaaacagcagcccacacacaaattctgaggagcagggactggctatgtcctgtgtccaccagtttaccttttccttgtgggccaagagttctgctttgtcccagagtttggttatgagagagagagaaaagacacatcgtttcttcagcctcaatgacaaaattagggaaaggcgttggaactacctagctgagctcagcatctgggagggtaaacccacaagaagaggcaagaacatcgccaaatcccattccttttcccgaggtacacgatgaaagtaacctccctagaacttggtgctaaattaaatcttccttcatccaatctaaagccgctgccctctcccatcattactgcccaggggtctgtttctgtggatggggtgccagcgcggctctgaaacctgaaaagccctggaggacccctccttctgagacctagcccacaccagcagtcctctcccacacgggggtctcttcctttgagacagtgggagcagatggggggctctgcgctggggaggcctcagctggatcctgccgggaagaaaaaagggagccaccgcggaggggggcagccccgccccggtccgccagctctgctgcggattggcccgatgtctctatatctgggatgccttcctggcacgaagctaccacgtccccagtgtctccacccgacgtc

SEQ ID NO: 39 - DNA, 186 bp, Mouse
ggtgagagcctcgaatgcttagaggggcggcgagggaggggactttgggagagggtcgggattcgggattttatggctggtcgctggggttctatgccatgtttcacgccgcgcgcccaaggcgcacgacggtttggctagtcgtgcgcccgacactggtccactaacaggctcccttcgcctaca

SEQ ID NO: 40 - DNA, 20 bp, Artificial Sequence
  Interspecific backcross primer 1
accaatgacccatgctccag

SEQ ID NO: 41 - DNA, 19 bp, Artificial Sequence
  Interspecific backcross primer 2
gcaggcagtaatgtggaca

SEQ ID NO: 42 - DNA, 29 bp, Artificial Sequence
  Primer A
tccaaagccgctgccctctcccatcatta

SEQ ID NO: 43 - DNA, 33 bp, Artificial Sequence
  Primer B
gcggaattcagacctagcccacaccagcagtcc

SEQ ID NO: 44 - DNA, 33 bp, Artificial Sequence
  Primer C
gcggaattcaccatggggggctctgcgctgggg

SEQ ID NO: 45 - DNA, 36 bp, Artificial Sequence
  Primer D
gcgaattcaccatgcagagatcgccgcccggctacg

SEQ ID NO: 46 - DNA, 22 bp, Artificial Sequence
  Primer E
caaagcgtggaaggagctgaac

SEQ ID NO: 47 - DNA, 30 bp, Artificial Sequence
  Primer F
gcgaattcctgggagccgggtctcttggtc

SEQ ID NO: 48 - DNA, 32 bp, Artificial Sequence
  Primer N
gcgaattcaccgggacccgagcttctgctacg

SEQ ID NO: 49 - DNA, 32 bp, Artificial Sequence
  Primer G
cagaattcaccgtcggcagtttggcgctctcc

SEQ ID NO: 50 - DNA, 29 bp, Artificial Sequence
  Primer H
gcggaattcctgtaggcgaagggagcctg

SEQ ID NO: 51 - DNA, 32 bp, Artificial Sequence
  Primer I
gcgaattcttatttagctccagcctccggacc

SEQ ID NO: 52 - DNA, 22 bp, Artificial Sequence
  Primer J
cagagtgggtagctcacggaag

SEQ ID NO: 53 - DNA, 30 bp, Artificial Sequence
  Primer K
gcgaattcagcagaagctcgggtcccgtgc

SEQ ID NO: 54 - DNA, 30 bp, Artificial Sequence
  Primer L
gcgaattcttatctagcctgagatgcaagc

SEQ ID NO: 55 - DNA, 30 bp, Artificial Sequence
  Primer M
taggccaccagctctaaaggctgttgcata

SEQ ID NO: 56 - DNA, 30 bp, Artificial Sequence; identical to SEQ ID NO: 53
  Transactivation assay primer 1

SEQ ID NO: 57 - DNA, 29 bp, Artificial Sequence
  Transactivation assay primer 2
gcgaattcctggagccgggtctcttggtc

SEQ ID NO: 58 - DNA, 7 bp, Artificial Sequence
  DNA motif recognized by all Sox members
aacaaag

SEQ ID NO: 59 - DNA, 21 bp, Artificial Sequence
  HMG box primer 1
ctggagccgggcctcttgctc

SEQ ID NO: 60 - DNA, 26 bp, Artificial Sequence
  HMG box primer 2
aattctatctagcctgagatgcaagc

SEQ ID NO: 61 - DNA, 22 bp, Artificial Sequence
  neo R primer
caagctcttcagcaatatcacg

SEQ ID NO: 62 - DNA, 22 bp, Artificial Sequence
  neo F primer
atctcctgtcatctcaccttgc

SEQ ID NO: 63 - DNA, 20 bp, Artificial Sequence
  Sox 18 box A primer
ccaacgtctcgcccacctcg

SEQ ID NO: 64 - DNA, 20 bp, Artificial Sequence
  Sox18 box B primer
gccgcttctccgccgtgttc

SEQ ID NO: 65 - DNA, 22 bp, Artificial Sequence
  neo R primer
caagctcttcagcaatatcacg

SEQ ID NO: 66 - DNA, 22 bp, Artificial Sequence
  neo F primer
atctcctgtcatctcaccttgc

SEQ ID NO: 67 - DNA, 20 bp, Artificial Sequence
  Lacz A primer
cagcacatccccctttcgcc

SEQ ID NO: 68 - DNA, 20 bp, Artificial Sequence
  Lacz B primer
ccaacgcagcaccatcaccg

SEQ ID NO: 69 - DNA, 20 bp, Artificial Sequence
  Sox18 A primer
ggctttccgggcaccctatg

SEQ ID NO: 70 - DNA, 20 bp, Artificial Sequence
  Sox18 B primer
aagcggtggagggcaaggac

SEQ ID NO: 71 - DNA, 20 bp, Artificial Sequence
  Sox18 box A primer
ccaacgtctcgcccacctcg

SEQ ID NO: 72 - DNA, 20 bp, Artificial Sequence
  Sox18 box B primer
gccgcttctccgccgtgttc

SEQ ID NO: 73 - DNA, 21 bp, Artificial Sequence
  5′ Sox18 A primer
tgagacagtgggagcagatgg

SEQ ID NO: 74 - DNA, 21 bp, Artificial Sequence
  5′ Sox18 B primer
gcaaagccaagtacggaggtc

SEQ ID NO: 75 - DNA, 18 bp, Artificial Sequence
  GAPDH F primer
tcggtgtgaacggatttg

SEQ ID NO: 76 - DNA, 18 bp, Artificial Sequence
  GAPDH R primer
attctcggccttgactgt

SEQ ID NO: 77 - DNA, 18 bp, Artificial Sequence
  GMUQ450 primer
gctcccttttttcttccc

SEQ ID NO: 78 - DNA, 20 bp, Artificial Sequence
  GMUQ480 primer
ggaaaaggaatgggatttgg

SEQ ID NO: 79 - DNA, 28 bp, Artificial Sequence
  GMUQ238 primer
gcgaattcctggagccgggcctcttgct

SEQ ID NO: 80 - DNA, 30 bp, Artificial Sequence; identical to SEQ ID NO: 53
  GMUQ239 primer

SEQ ID NO: 81 - DNA, 23 bp, Artificial Sequence
  GMUQ529 primer
gcgcggcctccctgtcaccaacg

SEQ ID NO: 82 - DNA, 23 bp, Artificial Sequence
  GMUQ530 primer
ccaaaggcggtgggaagaaggag

SEQ ID NO: 83 - DNA, 36 bp, Artificial Sequence; identical to SEQ ID NO: 45
  GMUQ401 primer

SEQ ID NO: 84 - DNA, 29 bp, Artificial Sequence
  GMUQ503 primer
gcggaattcctgtaggcgaagggagcctg

SEQ ID NO: 85 - DNA, 17 bp, Artificial Sequence
  hSOX18 primer A
gcccagaggagagcagc

SEQ ID NO: 86 - DNA, 17 bp, Artificial Sequence
  hSOX18 primer B
gcgccgcaagaagcagg

SEQ ID NO: 87 - DNA, 17 bp, Artificial Sequence
  hSOX18 primer C
gctgctctcctctgggc

SEQ ID NO: 88 - DNA, 17 bp, Artificial Sequence
  hSOX18 primer D
caggaggccgggctcca

SEQ ID NO: 89 - DNA, 17 bp, Artificial Sequence
  hSOX18 primer E
gcccgccgccgatctgt

SEQ ID NO: 90 - DNA, 17 bp, Artificial Sequence
  hSOX18 primer F
gcccgttccacctccca

SEQ ID NO: 91 - DNA, 17 bp, Artificial Sequence
  hSOX18 primer G
ccctacgcgcccaccga

SEQ ID NO: 92 - DNA, 17 bp, Artificial Sequence
  hSOX18 primer H
tcggtgggcgcgtaggg

SEQ ID NO: 93 - DNA, 17 bp, Artificial Sequence
  M13 forward primer
gtaaaacgacggccagt

SEQ ID NO: 94 - DNA, 17 bp, Artificial Sequence
  M13 reverse primer
caggaaacagctatgac

SEQ ID NO: 95 - DNA, 24 bp, Artificial Sequence
  SOX18 specific primer 1
ccgacgtggacctcaccgagttcg

SEQ ID NO: 96 - DNA, 24 bp, Artificial Sequence
  SOX18 specific primer 2
aggtggccagaagcccaggaaggg

SEQ ID NO: 97 - protein, 8 aa, Artificial Sequence
  Geysen library peptide
  MOD_RES (1)..(1): Acetylation
  PEPTIDE (1)..(2): Xaa is any amino acid
  PEPTIDE (3)..(4): Xaa is defined amino acid
  PEPTIDE (5)..(8): Xaa is any amino acid
XXXXXXXX

SEQ ID NO: 98 - protein, 378 aa, Mouse
MQRSPPGYGAQDDPPSRRDCAWAPGIGAAAEARGLPVTNVSPTSPASPSSLPRSPPRSPESGRYGFGRGERQTADELRIRRPMNAFMVWAKDERKRLAQQNPDLHNAVLSKMLGKAWKELNTAEKRPFVEEAERLRVQHLRDHPNYKYRPRRKKQERKVRRLEPGLLVPGLVQSSAPAEVFAAASGSARSFRELPTLGAEFDGLALPTPERRLLTGWSLAKSPFFPPALEALRTVLFGLSGHPMPLSWHGTRASATGRPWVKRSGQRRLPRHSQVSTMAPWATPGPFPNPLSPPPESPSLEGTEQLEPTADLWADVDLTEFDQYLNCSRTRPDATTLPYHVALAKLGPRAMSCPEESSLISALSDASSAVYYSACISG

SEQ ID NO: 99 - protein, 462 aa, Human
GRAPASPPSPQRSPPRSPEPGRYGLSPAARGERQAADESRIRRPMNAFMVWAKDERKRLAQQNPDLHNAVLSKMLGKAWKELNAAEKRPFVEEAERVCVQHLRDHPNYKYRPRRKKQARKARRLEPGLLLPGLAPPQPPPEPFPAASGSARAFRGSPAGAEFDGLGLPTPERSPLDGLEPGEAAFFPPPAAPRTARWRPSAPPTAHRVVAGPRRLLRGSPGGGAQDRAPRARSLACTTAPWARPARTPARCRRRPRPRRWRAPSPGARRRSVGRRGPHRVRPVPQLQPDSARRPRAPVPRGTGQTGPARHVLPRGEQPDSRCRTPAARSITARASRAAAPPGPCSASSRSPRDRSEPAVRSALSYACMFGSMSQPPRSQCCPCARSTSQATLPGLLATCLGGPLRGCLEFPRVPGLFQEARAQDLLAELPGLHFSTCSFSCSVFSTTRLYYFLLCPFKKYTS

SEQ ID NO: 100 - protein, 470 aa, Human
GRAPASPPSPQRSPPRSPEPGRYGLSPAGRGERQAADESRIRRPMNAFMVWAKDERKRLAQQNPDLHNAVLSKMLGKAWKELNAAEKRPFVEEAERLRVQHLRDHPNYKYRPRRKKQARKARRLEPGLLLPGLAPPQPPPEPFPAASGSARAFRELPPLGAEFDGLGLPTPERSPLDGLEPGEAAFFPPPAAPEDCALRPFRAPYAPTELSRDPGGCYGAPLAEALRTAPPAAPLAGLYYGTLGTPGPYPGPLSPPPEAPPLESAEPLGPAADLWADVDLTEFDQYLNCSRTRPDAPGLPYHVALAKLGPRAMSCPEESSLISALSDASSAVYYSACISGAAGAARVPAALPPAAPATDPTASLPLCSLIRVYVWFHVTAPEPVMLGLAPVPPPRPPFLGFWATCPRGAPARVPGVPTCPGAFPGSPSPGPVGRVARVTFLKHLLLFLQCIFYNQIVLIFFTLPFKIYLI

SEQ ID NO: 101 - DNA, 1225 bp, Mouse
catcagacctccgtacttggctttgcagtgcccgccactgtctcctgcgctcccgcgccgcgttccgcccaggccttgcccagctggaatgcagagatcgccgcccggctacggcgcacaggacgacccgccctcccgccgcgactgtgcatgggcccctggaatcggggccgctgctgaggcgcgcggcctccctgtcaccaacgtctcgcccacctcgcccgcctccccgtccagccttccgcggagcccaccgcgcagccccgaatcagggcgctatggctttggccgcggagagcgccaaactgccgacgagttgcgcattcggcggcccatgaacgccttcatggtgtgggcgaaggacgagcgcaagcgactggcgcaacaaaatccggatctgcacaacgcagtactgagcaagatgctgggcaaagcgtggaaggagctgaacacggcggagaagcggcccttcgtggaagaggccgaacggttgcgtgtgcagcacttgcgcgaccatcccaactacaagtaccggcctcgtcgtaaaaaacaggagcgcaaggtccggaggctggagccgggtctcttggtcccgggcctcgtgcagtcgtctgcgccggccgaggtcttcgctgcagcgtcagggtcagctcgctccttccgtgagctacccactctgggtgcggagttcgatgggttggcgctacccacgcccgagcgtcgtctcttgacgggctggagcctggcgaagtctcccttcttcccaccggctttggaggcccttaggactgtgctctttgggctttccgggcaccctatgcccctgagctggcacgggacccgagcttctgctacggggcgcccctgggtgaagcgctcaggacagcgccgcctgccgcgccactcgcaggtctctactatggcaccctgggccactccgggcccgtttcccaatcctctgtcaccaccacctgagtccccgtctcttgagggcacagagcaactggagcctaccgccgacctttgggccgatgtggacctcaccgaatttgaccagtatctcaattgcagccggactcgaccggatgccactacactcccctaccacgtggcactggccaaactaggtccgcgcgccatgtcctgtccagaagagagcagcctcatttctgcgctgtctgatgctagcagcgcggtctattacagtgcttgcatctcaggctag

SEQ ID NO: 102 - DNA, 1402 bp, Human
gggcgggcgcccgcctcgccgcccagcccgcagcgcagtcccccgcgcagccccgagccggggcgctatggcctcagcccggccgcccgcggggaacgccaggcggcagacgagtcgcgcatccggcggcccatgaacgccttcatggtgtgggcaaaggacgagcgcaagcggctggctcagcagaacccggacctgcacaacgcggtgctcagcaagatgctgggcaaagcgtggaaggagctgaacgcggcggagaagcggcccttcgtggaggaagccgaacgcgtgtgcgtgcagcacttgcgcgaccaccccaactacaagtaccggccgcgccgcaagaagcaggcgcgcaaggcccggcggctggagcccggcctcctgctcccgggattagcgcccccgcagccaccgcccgagcctttccccgcggcgtctggctcggctcgcgccttccgcggtagccccgctggcgccgagttcgacggcctggggctgcccacgcccgagcgctcgcctctggacggcctggagcccggcgaggctgccttcttcccaccgcccgcggccccgaggactgcgcgctggcgcccttccgcgccgcctaccgcgcaccgagttgtcgcgggaccccggcggttgctacggggctcccctggcggaggcgctcaggaccgcgccccccgcgcgcgctcgctggcctgtactacggcaccctgggcacgcccggcccgtaccccggcccgctgtcgccgccgcccgaggccccgccgctggagagcgccgagccctggggcccgccgccgatctgtgggccgacgtggacctcaccgagttcgaccagtacctcaactgcagccggactcggcccgacgcccccgggctcccgtaccacgtggcactggccaaactgggcccgcgcgccatgtcctgcccagaggagagcagcctgattcgcgctgtcggacgccagcagcgcggtctattacagcgcgtgcatctcgctaggccgccgcgccgccgggtccctgcagcgcttcctcccgcagcccccgcgaccgatccgagcccgccgtgcgctctgctctctcatacgcgtgtatgtttggttccatgtcacagccccctaggagccagtgatgctgcccttgcgcccgttccacctcccaggccacccttcctgggcttctggccacctgcctcggggggcccctgcgagggtgcctggagttcccacgtgtcccggggcttttccaggaagcccgagcccaggacctgttggcagagttgccagggttacatttttgaagcacctgctccttttcttgcagtgtattttctacaaccagattgtattaatattttttactttgcccttttaaaaaatatacctaatccc

SEQ ID NO: 103 - protein, 93 aa, Artificial Sequence
  Trans-activation domain sequence from SOX18M mutant polypeptide
LEPGLLVPGLVQPSAPPEAFAAASGSARSFRELPTLGAEFDGLGLPTPERSPLDGLEPGEASFFPPPLAPEDCALRAFRAPYAPELARDPSFC

SEQ ID NO: 104 - protein, 93 aa, Artificial Sequence
  Trans-activation domain sequence from SOX18 mutant polypeptide
LEPGLLVPGLVQPSAPPEAFAAASGAARAFRELPTLGAEFDGLGLPTPERSPLDGLEPGEASFFPPPLAPEDCALRAFRAPYAPELARDPSFC

SEQ ID NO: 105 - protein, 93 aa, Artificial Sequence
  Trans-activation domain sequence from SOX18 mutant polypeptide
LEPGLLVPGLVQPSAPPEAFAAASGSARFFRELPTLGAEFDGLGLPTPERSPLDGQEPGEASFFPPPLAPEDCALRAFRAPYAPELARDPSFC

SEQ ID NO: 106 - protein, 92 aa, Artificial Sequence
  Trans-activation domain sequence from SOX18 mutant polypeptide
LEPGLLVPGLVQPSAPPEAFAAASGSARSFRELPTLGAEFDGLGLPTPERSPLDGLEPGEASFFPPPLAPEDCALRAFRAPYAPELARDRLL

SEQ ID NO: 107 - protein, 86 aa, Artificial Sequence
  Trans-activation domain sequence from SOX18 mutant polypeptide
LEPGLLVPGLVQPSAPPEAFAAASGSARSFRELPTLGAEFDGLGLPTPERSPLDGLEPGEASLPGAGSLGPGHPMPLSWHGTRASA

SEQ ID NO: 108 - protein, 93 aa, Artificial Sequence
  Trans-activation domain sequence from SOX18 mutant polypeptide
LEPGLLVPGLVQPSAPPEAFAAASGSARSFRELPTLGAEFDGLGLPTPERSPLDGLEPGETSFFPPPLAPEDCALRAFRAPYAPELARDPSFC

SEQ ID NO: 109 - protein, 93 aa, Artificial Sequence
  Trans-activation domain sequence from SOX18 mutant polypeptide
LEPGLLVPGLVQPSAPPEAFAAASGSARSFRELPTLGAEFDGLGLPTPERSPLDGLEPGEVSFFPPPLAPEDCALRAFRAPYAPELARDPSFC

SEQ ID NO: 110 - protein, 93 aa, Artificial Sequence
  Trans-activation domain sequence from SOX18 mutant polypeptide
LEPGLLVPGLVQPSAPPEAFAAASGSARSFRELPTLGAEFDGLGLPTPERSPLDGLEPGEASFFPPPLAPEDCALRTFRAPYAPEMARDPSFC

SEQ ID NO: 111 - protein, 93 aa, Artificial Sequence
  Trans-activation domain sequence from SOX18 mutant polypeptide
LEPGLLVPGLVQPSAPPEAFAAASGSARSFRELPTLGAEFDGLGLPTPERSPMDGLEPGEASFFPPPLAPEDCALRAFRAPYAPELARDPSFL

SEQ ID NO: 112 - protein, 93 aa, Artificial Sequence
  Trans-activation domain sequence from SOX18 mutant polypeptide
LEPGLLVPGLVQPSAPPEAFAAASGSARSFRELLTLGAEFDGLGLPTPERSPLDGLEPGEASFFPPPLAPEDCALRAFRAPYAPELARDPSFW

SEQ ID NO: 113 - protein, 93 aa, Artificial Sequence
  Trans-activation domain sequence from SOX18 mutant polypeptide
LEPGLLVPGLVQPSAPPEAFAAASGSARSFRELPTLGAEFDGLGLPTPERSPLDGLEPGEASFFPPPLAPEDCAMRAFRAPYAPELARDPSFC

SEQ ID NO: 114 - protein, 93 aa, Artificial Sequence
  Trans-activation domain sequence from SOX18 mutant polypeptide
LEPGLLVPGLVQPSAPPEAFAAASGSARSFRELPTLGAEFDGLGLPTPERSPLDGLEPGEASFFPPPLAPGDCALRAFRAPYAPELARDPSFC

SEQ ID NO: 115 - protein, 93 aa, Artificial Sequence
  Trans-activation domain sequence from SOX18 mutant polypeptide
LEPGLLVPGLVQPSAPPGAFAAASGSARSFRELPTLGVEFDGLGLPTPERSPLDGLEPGEASFFPPPLAPEDCALRAFRAPYAPELARDPSFC

SEQ ID NO: 116 - protein, 93 aa, Artificial Sequence
  Trans-activation domain sequence from SOX18 mutant polypeptide
LDPGLLVPGLVQPSAPPEAFAAASGSARSFRELPTLGAEFDGLGLPTPERSPLDGLEPGEASFFPPPLAPEDCALRAFRAPYAPELARDPSFC

SEQ ID NO: 117 - protein, 93 aa, Artificial Sequence
  Trans-activation domain sequence from SOX18 mutant polypeptide
LEPGLLVPGLVQPSAPPEAFAAASGSARSFRELPTLGAEFDGLGLPTPERSSLDGLEPGEASFFPPPLAPEDCALRAFRAPYAPELARDPSFC

SEQ ID NO: 118 - protein, 93 aa, Artificial Sequence
  Trans-activation domain sequence from SOX18 mutant polypeptide
LEPGLLVPGLVQPSAPPEAFAAASGSARSFREIPTLGAEFDGLGIPTPERSPLDGLEPGEASFFPPPLAPEDCALRAFRAPYAPELARDPSFC

SEQ ID NO: 119 - protein, 93 aa, Artificial Sequence
  Trans-activation domain sequence from SOX18 mutant polypeptide
LEPGLLVPGLVQPSAPPEAFAAASGSARSFRELSTLGAEFDGLGLPTPERSPLDGLEPGEASFFPPPLAPEDCALRAFRAPYAPELARDPSFC

SEQ ID NO: 120 - protein, 93 aa, Artificial Sequence
  Trans-activation domain sequence from SOX18 mutant polypeptide
LEPGLLVPGLVQPSAPPEAFAAASGSTRSFRELPTLGAEFDGLGLPTPERSPLDGLEPGEASFFPPPLAPEDCALRAFRAPYAPELARDPSFC

SEQ ID NO: 121 - protein, 93 aa, Artificial Sequence
  Trans-activation domain sequence from SOX18 mutant polypeptide
LEPGLLVPGLVQPSAPPEAFVAASGSARSFRELPTLGAEFDGLGLPTPERSPLDGMEPGEASFFPPPLAPEDCALRAFRAPYAPELARDPSFC

SEQ ID NO: 122 - protein, 93 aa, Artificial Sequence
  Trans-activation domain sequence from SOX18 mutant polypeptide
LEPGLLVPGLVQPSAPPEAFAATSGSARSFRELPTLGAEFDGLGLPTPERSPLDGLEPGEASFFPPPLAPEDCALRAFRAPYAPELARDPSFC

SEQ ID NO: 123 - protein, 93 aa, Artificial Sequence
  Trans-activation domain sequence from SOX18 mutant polypeptide
LEPGLLVPGPVQPSAPPEAFAAASGSARSFRELPTLGAEFDGLGLPTPERSPLDGLEPGEASFFPPPLAPEDCALRAFRAPYAPELARDPSFC

SEQ ID NO: 124 - protein, 93 aa, Artificial Sequence
  Trans-activation domain sequence from SOX18 mutant polypeptide
LEPGLLVPGLVQPSAPPEAFAAASGSARSFRELPTLDAEFDGLGLPTPERSPLDGLEPGEASFFPPPLAPEDCALRAFRAPYAPELARDPSFC

SEQ ID NO: 125 - protein, 93 aa, Artificial Sequence
  Trans-activation domain sequence from SOX18 mutant polypeptide
LEPGLLVPGLVQPSAPPEAFAAASGSARSFRELPTLGAEFDGLGLPTLERSPLDGLEPGEASFFPPPLAPEDCALRAFRAPYAPELARDPSFC

SEQ ID NO: 126 - protein, 337 aa, Human
GRAPASPPSPQRSPPRSPEPGRYGLSPAGRGERQAADESRIRRPMNAFMVWAKDERKRLAQQNPDLHNAVLSKMLGKAWKELNAAEKRPFVEEAERLRVQHLRDHPNYKYRPRRKKQARKARRLEPGLLLPGLAPPQPPPEPFPAASGSARAFRELPPLGAEFDGLGLPTPERSPLDGLEPGEAAFFPPPAAPRTARWRPSAPPTAHRVVAGPRRLLRGSPGGGAQDRAPRARSLACTTAPWARPARTPARCRRRPRPRRWRAPSPGARRRSVGRRGPHRVRPVPQLQPDSARRPRAPVPRGTGQTGPARHVLPRGEQPDSRCRTPAARSITARASR

SEQ ID NO: 127 - protein, 93 aa, Artificial Sequence
  Activation domain sequence from SOX18 mutant polypeptide
LEPGLLVPGLVQPSAPPEAFAAASGSARSFRELPTLGAEFDGLGLPTPERSPLDGLEPGEAPSSHRLWRPRTALCGLSGHPMPLSWHGTRASA

SEQ ID NO: 128 - protein, 93 aa, Artificial Sequence
  Activation domain sequence from SOX18 mutant polypeptide
LEPGLLVPGLVQPSAPPEAFAAASGSARSFRELPTLGAEFDGLGLPTPERSPLDGLEPGEPPSSHRLWRPRTALCGLSGHPMPLSWHGTRASA

(19) United States
(12) Patent Application Publication
(10) Pub. No.: US 2002/0142415 A1
(43) Pub. Date: Oct. 3, 2002

(54) Novel polypeptides and polynucleotides and methods of using them

(76) Inventors: Peter Anthony Koopman, Queensland (AU); George Eugene Orlando Muscat, Queensland (AU)

Correspondence Address: HELLER EHRMAN WHITE & MCAULIFFE LLP, 1666 K STREET,NW, SUITE 300, WASHINGTON, DC, 20006, US

(21) Appl. No.: 09/814,777

(22) Filed: Mar. 23, 2001

(30) Foreign Application Priority Data
Mar. 24, 2000 (AU) PQ6457

(51) Int. Cl. C12Q 1/68; C07H 21/04; C12N 9/00; C12P 21/02; C12N 5/06; C07K 14/435

(52) U.S. Cl. 435/183; 435/6; 435/69.1; 435/325; 435/320.1; 536/23.2; 530/350

(57) ABSTRACT

Novel isolated Sox18 molecules are described for use in modulating cell differentiation, vasculogenesis, angiogenesis and/or hair follicle development, and in compositions for treating and/or preventing conditions that are associated, at least in part, with aberrant Sox18 expression or that are ameliorable, at least in part, by modulation of Sox18 expression as described hereinafter. The present invention also describes modulatory agents that modulate the expression of subgroup F Sox genes and to the use of these agents for prophylactic and/or therapeutic purposes. Further, the invention describes antigen-binding molecules that are immuno-interactive with the polypeptides of the invention.

### FIELD OF THE INVENTION

This invention relates generally to agents that modulate cell differentiation, vasculogenesis, angiogenesis and/or hair follicle development. More particularly, the present invention relates to novel SOX18 polypeptides that promote one or more of cell differentiation, vasculogenesis, angiogenesis and hair follicle development, and to polynucleotides encoding these novel polypeptides. The invention also relates to biologically active fragments of the novel SOX18 polypeptides as well as to variants and derivatives of these polypeptides. The invention further relates to the use of the polypeptides and polynucleotides of the invention in compositions for treating and/or preventing conditions that are associated with aberrant Sox18 expression or that are capable to be ameliorated by Sox18 expression. The invention also extends to modulatory agents that modulate Sox18 expression and to the use of these agents for prophylactic and/or therapeutic purposes. Further, the invention relates to antigen-binding molecules that are immuno-interactive with the polypeptides of the invention and to the use of these antigen-binding molecules for diagnostic purposes.

Bibliographic details of the publications referred to in this specification are collected at the end of the description.

### BACKGROUND OF THE INVENTION

Establishment and maintenance of a network of blood vessels is critical for the growth and survival of any complex organism. Vascular growth and remodelling is an important part of many physiological processes, such as embryogenesis, ovulation and the menstrual cycle, and a key component of many pathological conditions such as diabetic retinopathy, restenosis, wound healing and tumorigenesis. The control of vasculogenesis and angiogenesis has become one of the major issues in biomedical science, and is the focus of intense pharmaceutical interest.

The formation of blood vessels de novo during embryo development is known as vasculogenesis, and involves the differentiation of endothelial cells from mesenchymal precursors and their organisation into tubular vessels that subsequently recruit smooth muscle. The development of capillary networks through sprouting of existing vessels is known as angiogenesis; this process predominantly occurs in the embryo, but is also important during ovulation and the menstrual cycle, and a key component of many pathological conditions such as wound healing and tumorigenesis.

The roles of a number of genes and their protein products in the development and maintenance of blood vessels have been revealed through the study of gene knockouts in mice. These include vascular endothelial growth factor (VEGF) (Carmeliet, P., 1996) a well characterised inducer of angiogenesis; its tyrosine kinase receptors Flk1 (Shalaby, F. et al., 1995) and Flt1 (Fong, G. H. et al., 1995) involved in endothelial cell differentiation and organisation, respectively; the endothelial-specific tyrosine kinase angiopoietin-1 receptors Tie1 (Puri, M. C. et al., 1995) and Tie2 (otherwise known as Tek) (Dumont, D. J. et al., 1994) required for maintenance of blood vessel integrity and vascular remodelling, respectively.

Recently, it has been demonstrated that the MADS box transcription factor, MEF2C is expressed in developing endothelial cells and smooth muscle cells, as well as the surrounding mesenchyme, during embryogenesis. Targeted deletion of the mouse MEF2C gene resulted in severe vascular abnormalities and lethality in homozygous mutants by embryonic day 9.5. Endothelial cells were present and were able to differentiate, but failed to organise normally into a vascular plexus, and smooth muscle cells did not differentiate in mutant embryos. These vascular defects resemble those in mice lacking VEGF or its receptor Flt-1 (Lin, Q. et al., 1998).

We thus have some insights into the molecular pathways involved in vasculogenesis and angiogenesis. However, the transcriptional control mechanisms by which endothelial cell differentiation and function are achieved, are unclear.

The Sox (Sry-like HMG box) gene family takes its name from the first member isolated, the mammalian Y-linked testis-determining gene Sry (Gubbay, J. et al., 1990, Wright, E. M. et al., 1993). Sox genes are characterised by a conserved DNA sequence encoding a 79 amino acid “HMG” domain responsible for sequence-specific DNA binding. Several SOX proteins including SRY have been shown to bind to DNA, specifically to variations of the AACAAA/TG motif, and to activate transcription in vitro (Hosking, B M, et al., 1995; Ng, L J, et al., 1997; Schepers, G, et al., in press. Accepted January 2000), suggesting that SOX proteins function as transcriptional regulators. A large number of Sox genes have been identified to date, in diverse species such as human, mouse, chicken, frog and fruitfly (Bowles, J. et al 1996).

Expression studies, functional analysis through homologous recombination and mutational analyses in mice, and involvement of several Sox genes in human disease have illuminated their roles in development (reviewed in Wegner, M., 1999). For example, Sox9 is expressed in the developing skeleton (Wright, E M, et al., 1995), directly regulates the cartilage-specific genes Col2a1 (Ng, L J, et al., 1997; Bell, D M, et al., 1997), CD-RAP (Xie, W. F., et al., 1999) and aggrecan (Sekiya, I, et al., 2000), is essential for the differentiation of chondrocytes (Bi, W., et al., 1999), and is defective in patients with the skeletal syndrome campomelic dysplasia (Foster, J. W., et al., 1994; Wagner, T., et al., 1994). Sox10 expression is associated with neural crest cells in the mouse embryo, and mutations in SOX10 have been found in patients with the neurocristopathy Waardenburg-Shah syndrome (Kuhlbrodt, K., et al., 1998). The emerging picture is that Sox genes encode transcription factors with essential roles in directing cell fate determination and/or differentiation.

In the course of studying the Sox gene family, the present inventors isolated a Sox18 cDNA from a mouse heart cDNA library. Sequencing of these clones revealed an open reading frame of 1216 base pairs (bp) encoding a 378 amino acid (aa) polypeptide containing an HMG box. Northern analysis revealed abundant expression of a single 1.6-kilobase (kb) transcript in adult lung, skeletal muscle and heart (Dunn, T L, et al., 1995; W097/04090). Bacterially expressed SOX18 protein binds specifically to the DNA motif AACAAAG, recognised by all members of the SOX family characterised to date. It was also shown using GAL4 hybrid experiments, that SOX18 is a potent trans-activator of gene expression (Hosking, B M, et al., 1995; W097/0409).

### SUMMARY OF THE INVENTION

The present invention arises in part from the unexpected discovery that the open reading frame of murine Sox18 extends a further 273 bp upstream from that previously published and thus comprises an open reading frame of 1407 bp, encoding a 468 aa polypeptide. The inventors have also determined polynucleotide and polypeptide sequences relating to the human homologue of Sox18 (SOX18). A comparative sequence analysis between the mouse and human SOX18 polypeptides revealed a region of unusually high sequence conservation at the C-terminus (about 89% sequence identity) hereinafter referred to as the conserved C-terminal (CCT) domain, which is believed to represent a further, previously unrecognised functional domain of SOX18. The unusually high degree of conservation of this C-terminal region strongly argues for an important function associated therewith, perhaps in protein-protein interactions.

It has also been surprisingly discovered that mutations in the Sox18 gene underlie cardiovascular and hair follicle defects in ragged (Ra) mice. The inventors found in this regard that Sox18 is expressed in the developing vascular endothelium and hair follicles in mouse embryos. Further, no recombination was found between Sox18 and Ra in an interspecific backcross segregating for the Ra phenotype. The inventors also found point mutations in Sox18 from two different Ra alleles that result in mis-sense translation and premature truncation of the encoded protein. Interestingly, these mis-sense translations result in deletion of the CCT region referred to above, consistent with the hypothesis that this region of SOX18 has important function. Fusion proteins containing the mis-sense translation Ra mutations were also shown to have lost the ability to activate transcription relative to wild type controls in an in vitro assay. These observations implicate mutations in Sox18 as the underlying cause of the Ra phenotype, and identify Sox18 as a critical gene for cardiovascular and hair follicle formation. However, an analysis of Sox18^{-/-} mice produced by gene targeting has shown that despite the profound effects seen in Ra mice, Sox18^{-/-} mice have no obvious cardiovascular defects and only a mild coat defect with a reduced proportion of zigzag hairs. A reduction in the amount of pheomelanin pigmentation in hair follicles was also observed. Because of the mild effect of the mutation on the phenotype of Sox18^{-/-} M mice, it is believed that the semi-dominant nature of the Ra mutations is due to a trans-dominant negative effect mediated by the mutant SOX18 proteins rather than haploin sufficiency as has been observed for other SOX genes. Not wishing to be bound by any one particular theory or mode of action, the inventors believe that the mild phenotype of Sox18^{-/-} mice could be accounted for by the functional redundancy of SOX18 with SOX7 and SOX17, all of which belong to the F subgroup of SOX proteins on the basis of their sequence similarity and overlap of expression. Thus, it is predicted that cardiovascular and hair follicle formation could be modulated by increasing or decreasing the level and/or functional activity of each of the aforementioned subgroup F SOX proteins.

The inventors have also identified the myocyte-specific enhancer-binding factor 2 protein (MEF2C; Leifer et al., 1993) as a putative interacting partner protein for SOX18 during vascular development. In this respect, it was also found that mutant SOX18 proteins produced by Ra, RaJ, RaOp, and Ragl mice do not interact with MEF2C. This finding underscores the biological significance of the interaction between SOX18 and MEF2C, and further supports that Sox18 mutations in Ra mice act in a dominant-negative fashion.

It has also been discovered that Sox18 is expressed during angiogenesis in wound healing in adult mice and rats but is undetectable in unwounded skin. The inventors believe in this regard that Sox18 may represent a transcription factor involved in the induction of angiogenesis during wound healing and tissue repair, but not the maintenance of endothelial cells in undamaged tissue.

The inventors have reduced the above discoveries to practice in new isolated molecules for use in modulating cell differentiation, vasculogenesis, angiogenesis, hair follicle development, cell proliferation and/or tumorigenesis, and in compositions for treating and/or preventing conditions that are associated with aberrant Sox18 expression or that are capable of being ameliorated by Sox18 expression as described hereinafter.

Accordingly, in one aspect of the present invention, there is provided an isolated polypeptide, or a biologically active fragment thereof, or a variant or derivative of these, said polypeptide comprising the sequence set forth in any one of SEQ ID Nos. 2, 15 and 18, with the proviso that said fragment of SEQ ID NO. 2 comprises a contiguous sequence of amino acids contained within the sequence set forth in SEQ ID NO: 7.

In another aspect, the invention contemplates an isolated polypeptide, or a biologically active fragment thereof, or a variant or derivative of these, said polypeptide comprising the sequence set forth in SEQ ID NO: 18.

In yet another aspect, the invention features an isolated polypeptide, or a biologically active fragment thereof, or a variant or derivative of these, said polypeptide comprising the sequence set forth in SEQ ID NO: 15.

In still yet another aspect, the invention resides in an isolated polypeptide, or a biologically active fragment thereof, or a variant or derivative of these, said polypeptide comprising the sequence set forth in SEQ ID NO: 2, with the proviso that said fragment comprises a contiguous sequence of amino acids contained within the sequence set forth in SEQ ID NO: 7.

Preferably, the biologically active fragment comprises at least 6, more preferably at least 8, contiguous amino acids contained within the sequence set forth in any one of SEQ ID Nos. 2, 15 and 18. In a preferred embodiment of this type, the biologically active fragment is selected from residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 233-240, 241-248, 249-256, 257-264, 265-272, 273-280, 281-288, 289-296, 297-304, 305-312, 313-320, 321-328, 329-336, 337-344, 345-352, 353-360, 361-368, 369-376, 377-384, 385-392, 393-400, 401-408, 409-416, 417-424, 425-432, 433-440, 441-448, 449-456, 457-464, 261-468 of SEQ ID NO: 18.

In an alternate embodiment of this type, the biologically active fragment is selected from residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 233-240, 241-248, 249-256, 257-264, 265-272, 273-280, 281-288, 289-296, 297-304, 305-312, 313-320, 321-328, 329-336, 333-340 of SEQ ID NO. 15. In another embodiment of this type, the biologically active fragment is selected from residues 1-8, 9-16, 17-24, 25-32, 33-40, 41-48, 49-56, 57-64, 65-72, 73-80, 81-88, 84-91 of SEQ ID NO: 7.

Preferably, the biologically active fragment comprises a conserved C terminal (CCT) domain of SOX18. In a preferred embodiment of this type, the biologically active fragment comprises a contiguous sequence of amino acids at least 8 amino acids in length contained within SEQ ID NO: 30. In a preferred embodiment of this type, the biologically active fragment comprises the sequence set forth in SEQ ID NO: 30. In an alternate embodiment of this type, the biologically active fragment comprises the sequence set forth in SEQ ID NO. 13 or 27 In yet another embodiment of this type, the biologically active fragment comprises the sequence set forth in SEQ ID Nos. 28 or 29.

In another embodiment of the invention, the biologically active fragment, in addition to the CCT domain, further comprises a domain selected from the group consisting of the SOX18 HMG box domain and the SOX18 trans-activation domain, or variant of these. In a preferred embodiment of this type, the HMG box domain comprises the sequence set forth in SEQ ID NO: 9 or 23 and the trans-activation domain comprises the sequence set forth in SEQ ID NO: 11 or 25.

Preferably, the variant has at least 85%, preferably at least 90%, more preferably at least 95%, and still more preferably at least 98% sequence identity to the sequence set forth in any one of SEQ ID NO: 2, 15 and 18, or biologically active fragment thereof. In a preferred embodiment of this type, the variant is distinguished from the sequence set forth in any one of SEQ ID NO: 2, 15 and 18, or biologically active fragment thereof, by the substitution of at least one amino acid. In an especially preferred embodiment of this type, the substitution is a conservative substitution.

The variant may be obtained from any suitable animal, including mammals, birds and aqueous animals. Preferably, the variant is obtained from a mammal.

In yet another aspect, the invention provides an isolated polynucleotide encoding a polypeptide, fragment, variant or derivative as broadly described above. Preferably, the polynucleotide comprises the sequence set forth in any one of SEQ ID NO: 1, 3, 5, 14, 16, 17, 19 and 21, or a fragment thereof, or a polynucleotide variant of these.

Preferably, the polynucleotide fragment comprises at least 18, more preferably at least 24, contiguous nucleotides contained within the sequence set forth in any one of SEQ ID NO: 1, 3, 5, 14, 16, 17, 19 and 21.

Preferably, the polynucleotide fragment comprises a nucleic acid sequence encoding a SOX18 conserved C terminal (CCT) domain. In a preferred embodiment of this type, the polynucleotide fragment comprises a nucleic acid sequence encoding a contiguous sequence of amino acids contained within SEQ ID NO: 30 at least 8 amino acids in length. In a preferred embodiment of this type, the polynucleotide fragment comprises a nucleic acid sequence encoding the sequence set forth in SEQ ID NO: 30. Preferably, the polynucleotide fragment comprises a nucleic acid sequence encoding the sequence set forth in SEQ ID NO: 13 or 27. In a preferred embodiment of this type, the polynucleotide fragment comprises the sequence set forth in SEQ ID NO: 12 or 26. In an alternate embodiment, the polynucleotide fragment comprises a nucleic acid sequence encoding the sequence set forth in SEQ ID NO: 28 or 29.

Preferably, the polynucleotide fragment further comprises a nucleic acid sequence encoding a domain selected from the group consisting of the SOX18 HMG box domain and the SOX18 trans-activation domain, or variant of these. In a preferred embodiment of this type, the nucleic acid sequence encoding the HMG box domain encodes the sequence set forth in SEQ ID NO: 9 or 23 and the nucleic acid sequence encoding the trans-activation domain encodes the sequence set forth in SEQ ID NO: 11 or 25. In an especially preferred embodiment of this type, the nucleic acid sequence encoding the HMG box domain comprises the sequence set forth in SEQ ID NO: 8 or 22 and the nucleic acid sequence encoding the trans-activation domain comprises the sequence set forth in SEQ ID NO: 10 or 24.

In an alternate embodiment, the polynucleotide fragment comprises a contiguous sequence of nucleotides contained within the sequence set forth in any one of SEQ ID NO: 6, 38 and 39. The variant may be obtained from any suitable animal, including mammals, birds and aqueous animals. Preferably, the variant is obtained from a mammal.

In another aspect, the invention contemplates a vector comprising a polynucleotide as broadly described above.

In yet another aspect, the invention features an expression vector comprising a polynucleotide as broadly described above wherein the polynucleotide is operably linked to a regulatory polynucleotide.

In a further aspect, the invention provides a host cell containing a vector or expression vector as broadly described above.

The invention also contemplates a method of producing a recombinant polypeptide, fragment, variant or derivative as broadly described above, comprising:

culturing a host cell containing an expression vector as broadly described above such that said recombinant polypeptide, fragment, variant or derivative is expressed from said polynucleotide; and

isolating said recombinant polypeptide, fragment, variant or derivative.

In another aspect, the invention provides a method of producing a biologically active fragment as broadly described above, comprising:

contacting a MEF2C polypeptide with a fragment of a polypeptide as broadly described above; and

detecting the presence of a complex comprising the MEF2C polypeptide and the fragment, which indicates that said fragment is a biologically active fragment.

In a further aspect, the invention provides a method of producing a biologically active fragment as broadly described above, comprising:

introducing a fragment of the polypeptide or a polynucleotide from which the fragment can be translated into a cell; and

detecting an activity selected from the group consisting of cell differentiation, vasculogenesis, angiogenesis and hair follicle development, which indicates that said fragment is a biologically active fragment.

Preferably, said cell differentiation is endothelial cell differentiation.

In yet a further aspect, the invention provides a method of producing a polypeptide variant of a parent polypeptide comprising the sequence set forth in any one of SEQ ID NO: 2, 15 and 18, or a biologically active fragment thereof, comprising:

providing a modified polypeptide whose sequence is distinguished from the parent polypeptide by substitution, deletion or addition of at least one amino acid;

contacting a MEF2C polypeptide with the modified polypeptide; and

detecting the presence of a complex comprising the MEF2C polypeptide and the modified polypeptide, which indicates that said modified polypeptide is a variant.

In yet a further aspect, the invention provides a method of producing a polypeptide variant of a parent polypeptide comprising the sequence set forth in any one of SEQ ID NO: 2, 15, and 18, or a biologically active fragment thereof, comprising:

providing a modified polypeptide whose sequence is distinguished from the parent polypeptide by substitution, deletion or addition of at least one amino acid;

introducing said modified polypeptide or a polynucleotide from which the modified polypeptide can be translated into a cell; and

detecting modulation of an activity selected from the group consisting of cell differentiation, vasculogenesis, angiogenesis and hair follicle development, which indicates that said modified polypeptide is a polypeptide variant.

The present inventors have determined that down regulation or inactivation of SOX18 is associated, at least in part, with a reduction, abrogation or otherwise inhibition of an activity selected from the group consisting of cell differentiation, vasculogenesis, angiogenesis and hair follicle development. It has also been determined that such down regulation or inactivation is associated, at least in part, with promoting, augmenting or otherwise enhancing an activity selected from the group consisting of cell proliferation and tumorigenesis. They have also determined that, in order to more effectively modulate one or more of the aforementioned activities, it is desirable to also modulate other subgroup F SOX proteins, including, but not restricted to, SOX7 and SOX17. Accordingly, the isolated polypeptides and polynucleotides as broadly described above, together with SOX7 and SOX17 polypeptides and SOX7 and SOX17 polynucleotides can be used to provide both drug targets and regulators to modulate an activity selected from the group consisting of cell differentiation, vasculogenesis, angiogenesis, hair follicle development, cell proliferation and tumorigenesis and to provide diagnostic markers for one or more of these activities during normal or disease stages, e.g. using detectable polypeptides and polynucleotides as broadly described above, or using detectable agents which interact specifically with those polypeptides or polynucleotides.

Thus, in another aspect, the invention contemplates a method for modulating at least one activity selected from the group consisting of cell differentiation, vasculogenesis, angiogenesis and hair follicle development, said method comprising introducing into a cell a SOX18 polypeptide, fragment, variant or derivative as broadly described above or a polynucleotide from which said polypeptide, fragment, variant or derivative can be translated.

In a preferred embodiment, the method further comprises introducing into said cell a member selected from the group consisting of a SOX7 polypeptide, a SOX17 polypeptide, a biologically active fragment of said SOX7 or SOX17 polypeptide, a variant of said SOX7 or SOX17 polypeptide, a variant of said fragment, a derivative of said SOX7 or SOX17 polypeptide, a derivative of said fragment, a polynucleotide from which said SOX7 or SOX17 polypeptide, said fragment, said variant or said derivative can be translated.

Preferably, the SOX7 polypeptide comprises the sequence set forth in SEQ ID NO: 34, or variant thereof. Preferably the variant has at least 70%, more preferably at least 75%, even more preferably at least 80%, even more preferably at least 85%, even more preferably at least 90%, even more preferably at least 95%, and still even more preferably at least 98% sequence identity to the sequence set forth in SEQ ID NO: 34.

Preferably, the SOX17 polypeptide comprises the sequence set forth in SEQ ID NO: 36, or variant thereof Preferably the variant has at least 70%, more preferably at least 75%, even more preferably at least 80%, even more preferably at least 85%, even more preferably at least 90%, even more preferably at least 95%, and still even more preferably at least 98% sequence identity to the sequence set forth in SEQ ID NO: 36.

In a further aspect, the invention extends to a method of screening for an agent which modulates at least one activity selected from the group consisting of cell differentiation, vasculogenesis, angiogenesis, hair follicle development, cell proliferation and tumorigenesis, said method comprising:

contacting a preparation comprising a member selected from the group consisting of a SOX18 polypeptide, fragment, variant and derivative as broadly described above and a genetic sequence encoding said polypeptide, fragment, variant or derivative, with a test agent; and

detecting a change in the level and/or functional activity of said member or an expression product of said genetic sequence.

In yet another aspect, the invention encompasses a method of screening for an agent which modulates at least one activity selected from the group consisting of cell differentiation, vasculogenesis, angiogenesis, hair follicle development, cell proliferation and tumorigenesis, said method comprising:

contacting a preparation comprising a member selected from the group consisting of a SOX7 polypeptide, fragment, variant and derivative as broadly described above and a genetic sequence encoding said polypeptide, fragment, variant or derivative, with a test agent; and

detecting a change in the level and/or functional activity of said member or an expression product of said genetic sequence.

In still yet another aspect, the invention envisions a method of screening for an agent which modulates at least one activity selected from the group consisting of cell differentiation, vasculogenesis, angiogenesis, hair follicle development, cell proliferation and tumorigenesis, said method comprising:

contacting a preparation comprising a member selected from the group consisting of a SOX17 polypeptide, fragment, variant and derivative as broadly described above and a genetic sequence encoding said polypeptide, fragment, variant or derivative, with a test agent; and

detecting a change in the level and/or functional activity of said member or an expression product of said genetic sequence.

According to another aspect, the invention extends to an agent which modulates at least one activity selected from the group consisting of cell differentiation, vasculogenesis, angiogenesis, hair follicle development, cell proliferation and tumorigenesis, wherein said agent is obtained or identified by a screening method as broadly described above.

In another aspect of the invention, there is provided a method for modulating at least one activity selected from the group consisting of cell differentiation, vasculogenesis, angiogenesis, hair follicle development, cell proliferation and tumorigenesis, said method comprising introducing into said cell an agent as broadly described above for a time and under conditions sufficient to modulate the level and/or functional activity of SOX18.

In one embodiment, the agent increases the level and/or functional activity of SOX18. In this instance, the activity that is modulated is preferably selected from the group consisting of cell differentiation, vasculogenesis, angiogenesis, and hair follicle development.

In an alternate embodiment, the agent decreases the level and/or functional activity of SOX18. In such a case, the activity that is modulated is preferably cell proliferation or tumorigenesis.

In yet another aspect, the invention encompasses a method for modulating at least one activity selected from the group consisting of cell differentiation, vasculogenesis, angiogenesis, hair follicle development, cell proliferation and tumorigenesis, said method comprising introducing into said cell an agent as broadly described above for a time and under conditions sufficient to modulate the level and/or functional activity of SOX7.

In one embodiment, the agent increases the level and/or functional activity of SOX7. In this instance, the activity that is modulated is preferably selected from the group consisting of cell differentiation, vasculogenesis, angiogenesis, and hair follicle development.

In an alternate embodiment, the agent decreases the level and/or functional activity of SOX7. In such a case, the activity that is modulated is preferably cell proliferation or tumorigenesis.

In still yet another aspect, the invention features a method for modulating at least one activity selected from the group consisting of cell differentiation, vasculogenesis, angiogenesis, hair follicle development, cell proliferation and tumorigenesis, said method comprising introducing into said cell an agent as broadly described above for a time and under conditions sufficient to modulate the level and/or functional activity of SOX17.

In one embodiment, the agent increases the level and/or functional activity of SOX17. In this instance, the activity that is modulated is preferably selected from the group consisting of cell differentiation, vasculogenesis, angiogenesis, and hair follicle development.

In an alternate embodiment, the agent decreases the level and/or functional activity of SOX17. In such a case, the activity that is modulated is preferably cell proliferation or tumorigenesis.

In another aspect, the invention provides a composition for treatment and/or prophylaxis of at least one condition selected from the group consisting of artherosclerosis, restenosis, pulmonary disease, tissue injury and hair loss, comprising at least one member selected from the group consisting of a subgroup F SOX polypeptide, a biologically active fragment of said polypeptide, a variant of said polypeptide, a variant of said fragment, a derivative of said polypeptide, a derivative of said fragment, a polynucleotide from which said polypeptide, fragment, variant or derivative can be translated, and a modulatory agent that enhances the level and/or functional activity of said polypeptide, together with a pharmaceutically acceptable carrier.

In yet another aspect, the invention resides in a composition for treatment and/or prophylaxis of tumorigenesis, comprising one or more agents that reduce the level and/or functional activity of at least one subgroup F SOX polypeptide, together with a pharmaceutically acceptable carrier.

In a preferred embodiment, the subgroup F SOX polypeptide is selected from SOX7, SOX17 and SOX18.

Preferably, the composition comprises one or more members that enhance the level and/or functional activity of two, and preferably all, of the subgroup F SOX polypeptides. In a preferred embodiment of this type, the composition comprises one or more members that enhance the level and/or functional activity of two, and preferably all, of SOX7, SOX17 and Sox18.

According to another aspect of the invention, there is provided a method for treatment and/or prophylaxis of at least one condition selected from the group consisting of artherosclerosis, restenosis, pulmonary disease, tissue injury and hair loss, said method comprising administering to a patient in need of such treatment a therapeutically effective amount of at least one member selected from the group consisting of a subgroup F SOX polypeptide, a biologically active fragment of polypeptide, a variant of said polypeptide, a variant of said fragment, a derivative of said polypeptide, a derivative of said fragment, a polynucleotide from which said polypeptide, fragment, variant or derivative can be translated, and a modulatory agent that enhances the level and/or functional activity of said polypeptide, and optionally together with a pharmaceutically acceptable carrier.

According to another aspect of the invention, there is provided a method for treatment and/or prophylaxis of tumorigenesis, comprising administering to a patient in need of such treatment a therapeutically effective amount of one or more agents that reduce the level and/or functional activity of at least one subgroup F SOX polypeptide, and optionally together with a pharmaceutically acceptable carrier.

In another aspect, the invention resides in the use of a SOX18 polypeptide, fragment, variant or derivative according to the present invention to produce an antigen-binding molecule that is specifically immuno-interactive with said polypeptide, fragment, variant or derivative.

In yet another aspect, the invention provides antigen-binding molecules so produced.

In another aspect, the invention envisions a method for detecting a specific polypeptide or polynucleotide sequence, comprising detecting a sequence of:

SEQ ID NO: 2, or a fragment thereof, at least 6 amino acids residues in length, comprising a contiguous sequence of amino acids contained within the sequence set forth in SEQ ID NO: 7; or

SEQ ID NO: 1, 3 or 5, or a fragment thereof, at least 18 nucleotides in length comprising a contiguous sequence of nucleotides contained within the sequence set forth in SEQ ID NO: 6; or

SEQ ID NO: 18, or a fragment thereof at least 6 amino acids residues in length; or

SEQ ID NO: 17, 19 or 21, or a fragment thereof at least 18 nucleotides in length; or

SEQ ID NO: 15, or a fragment thereof at least 6 amino acids residues in length; or

SEQ ID NO: 14, or 16, or a fragment thereof at least 18 nucleotides in length.

In yet another aspect, there is provided a method for detecting a polypeptide, fragment, variant or derivative as broadly described above, comprising:

detecting expression in a cell of a polynucleotide encoding said polypeptide, fragment, variant or derivative.

In yet another aspect of the invention, there is provided a method of detecting a polypeptide, fragment, variant or derivative as broadly described above, comprising:

contacting a test polypeptide with a MEF2C polypeptide; and

detecting the presence of a complex comprising said MEF2C polypeptide and said polypeptide, fragment, variant or derivative in said contacted sample.

According to another aspect of the invention, there is provided a method of detecting in a biological sample a polypeptide, fragment, variant or derivative as broadly described above, comprising:

contacting the sample with an antigen-binding molecule as broadly described above; and

detecting the presence of a complex comprising said antigen-binding molecule and said polypeptide, fragment, variant or derivative in said contacted sample.

In yet another aspect, there is provided a method for detecting a polypeptide, fragment, variant or derivative as broadly described above, comprising:

detecting expression in a cell of a polynucleotide encoding said polypeptide, fragment, variant or derivative.

In yet another aspect, the invention encompasses a method for detecting an activity selected from the group consisting of cell differentiation, vasculogenesis, angiogenesis and hair follicle development, comprising

detecting in a biological sample an expression product from at least one subgroup F SOX polynucleotide.

The invention also encompasses the use of the polypeptide, fragment, variant or derivative as well as the modulatory agents as broadly described above in the study, and modulation of cell differentiation, vasculogenesis, angiogenesis, hair follicle development, cell proliferation and tumorigenesis.

### BRIEF DESCRIPTION OF THE DRAWINGS

FIG. 1: Sequence alignment of the novel mouse SOX18 polypeptide with the previously published SOX18 polypeptide. The novel sequence is shown above the known sequence. Asterisks indicate identical amino acids and periods indicate homologous amino acids at corresponding positions in the polypeptide sequence. Hyphens indicate gaps in the sequence to maximise alignment between the two polypeptide sequences.

FIG. 2: Sequence alignment of the novel mouse Sox18 polypeptide with the partial human Sox18 polypeptide. The mouse sequence is shown above the human sequence. Asterisks, periods and hyphens have the same meaning as for FIG. 1.

FIG. 3: Sequence alignment of the published HAF-2 polypeptide with the partial human SOX18 polypeptide. The HAF-2 sequence is shown above the SOX18 sequence. Asterisks, periods and hyphens have the same meaning as for FIG. 1.

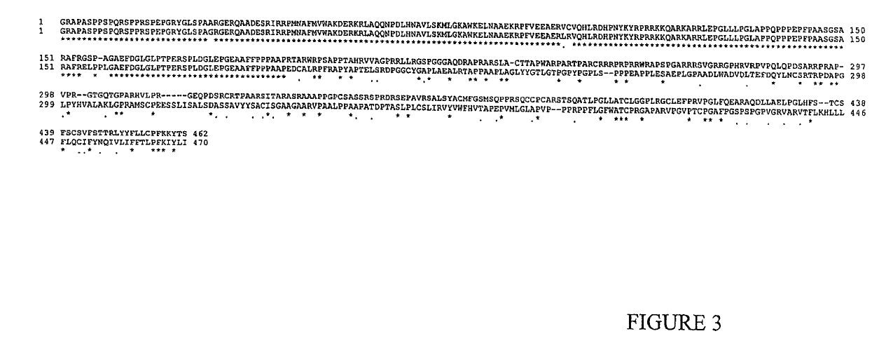

FIG. 4: Sequence alignment of the novel mouse Sox18 polynucleotide with the previously published Sox18 polynucleotide. The novel sequence is shown above the known sequence. Asterisks indicate identical nucleotides at corresponding positions in the polynucleotide sequence. Hyphens indicate gaps in the sequence to maximise alignment between the two polynucleotide sequences.

FIG. 5: Sequence alignment of the published HAF-2 polynucleotide with the partial human Sox18 polynucleotide. The HAF-2 sequence is shown above the Sox18 sequence. Asterisks and hyphens have the same meaning as for FIG. 4.

FIG. 6: Sox18 expression in the developing mouse cardiovascular system. a, Posterior view of a 7.5 dpc embryo revealing Sox18 expression in the allantois (al) and blood islands (bi) of the yolk sac. b, Expression continues in the blood islands and allantois (al) of an 8.0 dpc embryo and is evident in cells fated to become the endocardium (end). The plane of section in FIG. 6j is indicated by a red line. c, At 8.5 dpc expression persists in the allantois (al) and the nascent vasculature of the yolk sac (ys) and is evident in the paired dorsal aortae (da) and heart (ht). d, Sox18 expression is detected in Flt1−/− embryos in a pattern reflecting the disturbed blood vessel development but is absent in Flk1−/− embryos (e). f and g, 9.5 dpc embryos showing Flk1 and Sox18 expression respectively. In both cases expression is detected in the intersomitic vessels (isv) along with the network of smaller vessels in the head and trunk mesenchyme. The planes of section for FIGS. 6k-m are indicated by red lines. h, 11.0 dpc embryo showing Sox18 expression in the intersomitic vessels and the branching network of smaller vessels formed by angiogenesis. i, At 12.5 dpc Sox18 expression is detected in branching vessels on the surface of the abdomen and between the somites. Further, high levels of Sox18 expression are evident the nascent vibrissae follicles (vf). j, section of an 8.0 dpc embryo showing Sox18 expression in the yolk sac mesoderm (ysm), the endothelial cells around the foregut (fg) and in the presumptive endocardial cells (end) of the cardiogenic plate. The neural folds (nf) are indicated. Posterior, p; anterior, a. Scale bar, 100 μM. k, section of a 9.5 dpc mouse embryo showing Sox18 expression in the endothelial cells of the paired dorsal aortae (da). Red blood cells can be seen in the lumens of these vessels. The spinal cord (sc) is labelled. Dorsal, d; ventral, v. Scale bar, 50 μM. l, section of a 9.5 dpc mouse embryo showing Sox18 expression in the endothelial cells of the intersomitic vessels (isv) and the yolk sac mesoderm (ysm). Dorsal, d; ventral, v. Scale bar, 100 μM. m, Higher magnification of a 9.5 dpc mouse embryo section showing Sox18 expression in the endothelial cells of an intersomitic vessel. Scale bar, 20 μM.

FIG. 7: Sox18 expression in developing hair follicles. a, Sox18 expression in the vibrissae follicles (vf) and pelage follicles (p) of a 14.0 dpc embryo. b, c and d show cross sections through the vibrissae follicles of the face of 14.0 dpc embryos. b, Sox18 expression can be seen in the underlying mesenchyme of follicles at all stages of maturity; different stages of follicle development are present simultaneously in this section, with caudal vibrissae follicles being more mature and rostral follicles less mature. c, Shh expression is confined to the invaginating epithelium. d, Ptc1 expression is detected in the invaginating epithelium and the surrounding mesenchyme. e, diagrammatic representation of the domains of gene expression in the developing follicle with Sox18 expression shown in blue, Shh in red and Ptc1 in yellow.

FIG. 8: Mutations in Sox18^{Ra} and Sox18^{RaJ}. a, sequencing chromatograms show the deletion of a cytosine residue at nucleotide position 960 in Sox18^{Ra} and the deletion of a guanine residue at nucleotide position 959 in Sox18^{RaJ}. Wild type Sox18 sequence here is represented by C3H/HeSnJ, the background strain upon which RaJ is maintained. b, the mutation in the Sox18^{Ra}, ORF introduces a frame shift and mis-sense coding from amino acid 314 until a premature stop at position 435. Similarly, the mutation in Sox18^{RaJ} introduces a frame shift in the ORF and mis-sense coding from amino acid position 313 until the premature stop at position 435. The mis-sense amino acids are shown in red type in the translation. c, diagrammatic representations of the deduced protein products of Sox18, Sox18^{Ra} and Sox18^{RaJ} . The numbering indicates the amino acid co-ordinates of the represented boxes. The stippled boxes indicate mis-sense coding in the mutant Sox18 ORFs before the premature stop at position 435.

FIG. 9: Transcriptional trans-activation by wild type and mutant SOX18 proteins in a GAL4-fusion assay. CAT activity of GAL0 indicates the basal level of CAT reporter gene activity. A fusion protein of the GAL4 DNA-binding domain and the wild type SOX18 trans-activation domain (GAL-Sox18^{WT}) showed high levels of CAT activity. In contrast, fusion proteins of the GAL DNA-binding domain and SOX18 trans-activation domain from both the Ra and Ra^{J} mice (GAL-Sox18^{Ra} and GAL-Sox18^{RaJ}) failed to activate transcription of the reporter gene above basal levels. CAT activity is expressed as a percentage of GAL-Sox18^{WT} activity. Error bars represent standard deviation.

FIG. 10: shows an alignment of trans-activation domain sequences from different SOX18 mutant polypeptides and their respective transcriptional trans-activation activities.

FIG. 11: shows an alignment of trans-activation domain sequence portions from Sox18^{WT}, Sox18^{Ra}, and Sox18^{Raj} and the transcriptional trans-activation activities corresponding to these proteins as well as other mutant proteins.

FIG. 12: Expression of Sox18 during wound healing. Silver grains (black) corresponding to Sox18 mRNA overlie nascent capillaries in granulation tissue (arrowed).

FIG. 13: Sox18 mutations in ragged mice. A, Chromatograms showing the single base deletions in Sox18^{Ragl} (left) and Sox18^{Raop} (right), compared to wild-type Sox18 (Sox18WT). Nucleotide numbering is shown. Arrow indicates the position of the deleted base. B, Wild-type and mutant nucleotide and amino acid sequence. Amino acid residues printed in red represent missense coding. Amino acid numbering is shown. C, Schematic representation of the mutant SOX18 proteins compared to that of the wild-type protein. Missense coding is represented by red boxes.

FIG. 14: In vitro trans-activation by mutant and wild-type SOX18. Results are expressed as relative light units (RLU) and compared to GALO, which indicates the basal level of LUC reporter gene activity. Error bars represent standard deviation of triplicate assays. Equivalent expression of all GAL4-fusion proteins was confirmed by Western blot (data not shown).

FIG. 15: Disruption of Sox18 by gene targeting. a, Structure of Sox18 and gene targeting by homologous recombination. The targeting vector was designed to replace the regions of exon 1 and 2 (X1 and X2) encoding the HMG box domain (cross-hatched boxes) with a LacZ reporter cassette (LacZ) and neo^{r} selection cassette (neo). The probe was designed for screening for homologous recombinants in ES cells by HindIII digestion of genomic DNA. pA, poly-adenylation sites, TK, thymidine kinase cassette, Xb, XbaI and H, HindIII restriction sites. b, Detection of homologous recombinants in ES cells. Southern blotting of G418-resistant genomic DNA digested with HindIII (using the external probe, left) reveals a wild-type band of approximately 12 kb and a targeted band of 8.5 kb as expected. Probing the same Southern blot with an internal probe (LacZ fragment, right) reveals a targeted band of 8.5 kb, as expected, indicating that there was a single homologous recombination event. c, Genotyping of Sox18^{-/-} mice by Southern blotting. The same strategy was used as for screening ES cells, left, revealing the genotype of wild-type, heterozygous and homozygous knock out mice. Probing the same Southern blot with a probe specific to the HMG box-encoding region (right) confirmed that this region was removed in the targeted loci.

FIG. 16: RT-PCR analysis of transcripts from the Sox18^{-/-} and wild-type loci. 9.5 dpc littermates were used to provide RNA samples. RT-PCR of a region 5′ to the HMG box domain expected to be in both the wild-type and mutant transcripts (labeled 5′), RT-PCR of a portion of the LacZ reporter, expected to be present only in mutant transcripts (labeled LacZ), RT-PCR spanning the HMG box region and 186 bp intron (labeled HMG box). This also served as a control for genomic DNA contamination. Genomic bands would be 453 bp in this reaction. RT-PCR of a portion of the neo^{r} marker, expected to be present only in mutant transcripts (labeled neo) and RT-PCR of a region of the trans-activation domain (labeled 3′).

FIG. 17: Analysis of vascular development in Sox18^{-/-} embryos at 9.5 dpc. a, and b, PECAM-1 (CD31) immuno-staining of Sox18^{+/+} and ^{-/-} embryos respectively, showing staining of the aortic arches (aa), atrial chamber (ac), dorsal aorta (da), outflow tract (oft) and ventricular chamber (vc). c, and d, The embryos shown in a, and b, respectively, showing staining of the intersomitic vessels (isv) and the vasculature of the limb bud mesenchyme (lbm).

FIG. 18: Gross morphology and vibrissae formation in Sox18^{-/-} embryos. a, and b, Gross morphology of Sox18^{+/+} and Sox18^{-/-} embryos respectively at 14.5 dpc. c, and d, Close-up view of the embryos in a and b, respectively, showing the grossly visible vibrissae follicle (vf) formation.

FIG. 19: Coat color of Sox18^{-/-} mice. The mice shown in are the same two male, agouti littermates. a, Sox18^{+/+} and Sox18^{-/-} littermates at 8 days post natal. Note the darker color of the Sox18^{-/-} sibling. b, Sox18^{+/+} and Sox18^{-/-} littermates at 2 months post natal; the color difference is less pronounced than at 8 days post natal. c, and d, Vibrissae of the Sox18^{+/+} and Sox18^{-/-} littermates respectively at 3 months post natal showing no obvious differences.

FIG. 20: Pigmentation of hair types in Sox18^{-/-} mice. The hair samples shown are from agouti littermates. a, and b, Sub-apical pheomelanin banding in auchenes from Sox18^{'/+} and Sox18^{-/-} mice respectively. Arrows indicate pheomelanin bands. Arrowhead indicates a greatly reduced pheomelanin band. Scale bars, 200 μm. c, and d, Sub-apical pheomelanin banding in zigzags from Sox18^{+/+} and Sox18^{-/-} mice respectively. The arrow indicates a pheomelanin band. The arrowhead indicates a greatly reduced pheomelanin band. Note the absence of any pheomelanin in the zigzag on the right in d. Scale bars, 100 μm. e, and f, Awls from Sox18^{+/+} and Sox18^{-/-} mice respectively. Note the presence of a sub-apical pheomelanin band in an awl from a Sox18^{+/+} mouse (indicated by an arrow), but not in awl from a Sox18^{-/-} mice. Scale bars, 1 mm.

FIG. 21: Survey of the prevalence of hair types in Sox18^{-/-} mice. Note the reduced proportion of zigzag hairs amongst the coat of Sox18^{-/-} mice. Error bars represent standard deviation.

FIG. 22: The finalized gene and deduced amino acid sequence of Sox18. The A nucleotide of the ATG codon that defines the start of translation is designated as number 1. The dashed double underlined nucleotide sequence denotes the intron present within the HMG box at precisely the position in all published Sox genes from the sub-group D and Sox17 in the F sub-group. An in-frame stop codon, denoted by the bold star at nucleotide position 751, is encoded within the intron. The single dashed underline denotes the section of Sox18 empirically determined to encompass the trans-activation domain.

FIG. 23: Comparison of intron positions present in the HMG-box of the sub-groups F and G. The HMG-box is represented by the black box. The amino acid position of the intron relative to the start of the HMG-box is given above each protein.

FIG. 24: A comparison of the nucleotide (A) and the amino acid (B) sequence of the original, published cDNA sequence (Sox18 orig) and the gene sequence of the ORF for Sox18 (Sox18 ORF). The solid underline denotes the DNA binding/HMG box which contains three silent nucleotide changes. Each, interestingly, is a change from T to C. The dashed underline denotes the trans-activation domain and contains 20 nucleotide changes and four frame-shifts.

FIG. 25: Protein sequence comparison between Sox 18 and the deduced proteins of HAF-2. Haf2-2 is the protein translated from the modified DNA sequence of HAF-2 after comparison with Sox18 and insertion of gaps (no other changes to the DNA sequence were allowed). Haf2-1 is the original deduced protein sequence from Stevens et al., (1996). The solid underline denotes the HMG box, while the dashed underline denotes the trans-activation domain of murine Sox18.

FIG. 26: Analysis of the Sox18 activation domain The activity of wild type and mutant Sox 18 activation domains. (A) and (B) COS-1 cells were co-transfected with pG5E1bCAT reporter (3.0 mg) and the GAL4-Sox18 wild type and mutant activation domains and assayed for CAT activity 48±72 h after transfection as described previously. Results shown are mean ± SD and were derived from three independent experiments. (C) The positions of the mutations in the Sox18 activation domain are denoted by bold type, while those amino acids that were unchanged are indicated by a dash.

FIG. 27: Analysis of the Sox18 expression profile via RT-PCR, in various adult mouse tissues. Total RNA was isolated, 5 mg was subjected to reverse transcription with Superscript II and a maximum of 1/10 of the reaction used for PCR. The quantity of cDNA added to the PCR was controlled for by HPRT (giving a band of 380 bp) which has ubiquitous expression. All cDNA samples were amplified in parallel with equivalent amounts of RNA as a control for genomic DNA contamination. The number of cycles of amplification appears on the left of the figure and emphasizes the fact that semi-quantitation of the RT-PCR products could occur before the saturation point of the amplification process.

FIG. 28: Sequence and genomic organization of human SOX18. a, Alignment of the deduced peptide sequences of mouse (m) and human (h) SOX18 and HAF-2. Identical amino acid residues are indicated by dark shading and conservative amino acid changes by light shading. The region of the HMG box domain is indicated by a solid bar and the mouse SOX18 trans-activation domain by a broken bar above the peptide sequence. Arrowheads indicate the site of the intron in mouse and human SOX18. Accession numbers are: mouse Sox18, AF288518; human Sox18, AF270652. Sequence in lower case is from a genomic clone (AL356790). Asterisk indicates in-frame stop codon. Closed circle marks the first methionine residue in the human SOX18 open reading frame. b, Splice donor and acceptor sites associated with the 196 bp intron of human Sox18. Upper case letters indicate coding nucleotides, lower case letters indicate non-coding nucleotides and bold letters indicate the deduced amino acid residues. Arrowheads indicate the splice sites.

FIG. 29: Northern blot analysis showing Sox18 expression in various human tissues. Br, brain; H, heart; SkM, skeletal muscle; Co, colon (non-mucosal); Th, thymus; Sp, spleen; K, kidney; Li, liver; SI, small intestine; Pl, placenta; Lu, lung; Leu, peripheral blood leukocyte. The transcripts are marked by arrowheads and size markers by arrows. The probe used for Sox18 expression was a fragment from nucleotide 949 to 1730 of EST A1744846. The northern filter was probed with β-actin as a control for RNA loading.

FIG. 30: Chromosomal localization of Sox18 by radiation hybrid mapping, 22.44 cR distal to the marker D20s173.

### BRIEF DESCRIPTION OF THE SEQUENCES: SUMMARY TABLE

TABLE A

| SEQUENCE ID | | |
|---|---|---|
| NUMBER | SEQUENCE | LENGTH |
| SEQ ID NO: 1 | Full-length mouse Sox18 genomic sequence | 3472 bases |
| SEQ ID NO: 2 | Full-length mouse SOX18 polypeptide sequence | 468 residues |
| SEQ ID NO: 3 | Mouse Sox18 exonic sequence (intron removed) | 3286 bases |
| SEQ ID NO: 4 | Mouse SOX18 polypeptide encoded by SEQ ID NO: 3 | 468 residues |
| SEQ ID NO: 5 | Mouse Sox18 CDS | 1407 bases |
| SEQ ID NO: 6 | Novel murine Sox18 5′ CDS | 273 bases |
| SEQ ID NO: 7 | Polypeptide encoded by SEQ ID NO: 6 | 91 residues |
| SEQ ID NO: 8 | Polynucleotide sequence encoding mouse HMG Box | 237 bases |
| SEQ ID NO: 9 | Polypeptide encoded by SEQ ID NO: 8 | 79 residues |
| SEQ ID NO: 10 | Polynucleotide sequence encoding mouse trans- | 282 bases |
| | activation domain | |
| SEQ ID NO: 11 | Polypeptide encoded by SEQ ID NO: 10 | 94 residues |
| SEQ ID NO: 12 | Polynucleotide sequence encoding murine CCT domain | 264 bases |
| SEQ ID NO: 13 | Polypeptide encoded by SEQ ID NO: 12 | 88 residues |
| SEQ ID NO: 14 | Partial human SOX18 cDNA | 1421 bases |
| SEQ ID NO: 15 | Polypeptide encoded by SEQ ID NO: 14 | 340 residues |
| SEQ ID NO: 16 | Partial human SOX18 CDS | 1023 bases |
| SEQ ID NO: 17 | Full-length human SOX18 genomic sequence | 1919 bases |
| SEQ ID NO: 18 | Human SOX18 polypeptide encoded by SEQ ID NO: 17 | 384 residues |
| SEQ ID NO: 19 | Full-length human SOX18 cDNA | 1730 bases |
| SEQ ID NO: 20 | Human SOX18 polypeptide encoded by SEQ ID NO: 19 | 384 residues |
| SEQ ID NO: 21 | Full-length human SOX18 CDS | 1155 bases |
| SEQ ID NO: 22 | Polynucleotide sequence encoding human HMG Box | 237 bases |
| SEQ ID NO: 23 | Polypeptide encoded by SEQ ID NO: 22 | 79 residues |
| SEQ ID NO: 24 | Polynucleotide sequence encoding human trans- | 282 bases |
| | activation domain | |
| SEQ ID NO: 25 | Polypeptide encoded by SEQ ID NO: 24 | 94 residues |
| SEQ ID NO: 26 | Polynucleotide sequence encoding human CCT domain | 264 bases |
| SEQ ID NO: 27 | Polypeptide encoded by SEQ ID NO: 26 | 88 residues |
| SEQ ID NO: 28 | CCT domain motif I | 9 residues |
| SEQ ID NO: 29 | CCT domain motif II | 6 residues |
| SEQ ID NO: 30 | CCT domain consensus | 88 residues |
| SEQ ID NO: 31 | Chicken Sox18 cDNA | 1593 bases |
| SEQ ID NO: 32 | Polypeptide encoded by SEQ ID NO: 31 | 418 residues |
| SEQ ID NO: 33 | Mouse Sox7 cDNA | 3266 bases |
| SEQ ID NO: 34 | Polypeptide encoded by SEQ ID NO: 33 | 380 residues |
| SEQ ID NO: 35 | Mouse Sox17 cDNA | 1512 bases |
| SEQ ID NO: 36 | Polypeptide encoded by SEQ ID NO: 35 | 419 residues |
| SEQ ID NO: 37 | Putative Sox18 ligand binding site | 26 residues |
| SEQ ID NO: 38 | Novel mouse Sox18 5′ genomic sequence | 1701 bases |
| SEQ ID NO: 39 | Mouse Sox18 intronic sequence | 186 bases |
| SEQ ID NO: 40 | Interspecific backcross primer 1 | 20 bases |
| SEQ ID NO: 41 | Interspecific backcross primer 2 | 19 bases |
| SEQ ID NO: 42 | Primer A | 29 bases |
| SEQ ID NO: 43 | Primer B | 33 bases |
| SEQ ID NO: 44 | Primer C | 33 bases |
| SEQ ID NO: 45 | Primer D | 36 bases |
| SEQ ID NO: 46 | Primer E | 22 bases |
| SEQ ID NO: 47 | Primer F | 30 bases |
| SEQ ID NO: 48 | Primer N | 32 bases |
| SEQ ID NO: 49 | Primer G | 32 bases |
| SEQ ID NO: 50 | Primer H | 29 bases |
| SEQ ID NO: 51 | Primer I | 32 bases |
| SEQ ID NO: 52 | Primer J | 22 bases |
| SEQ ID NO: 53 | Primer K | 30 bases |
| SEQ ID NO: 54 | Primer L | 30 bases |
| SEQ ID NO: 55 | Primer M | 30 bases |
| SEQ ID NO: 56 | Transactivation assay primer 1 | 30 bases |
| SEQ ID NO: 57 | Transactivation assay primer 2 | 29 bases |
| SEQ ID NO: 58 | DNA motif recognised by all Sox members | 7 bases |
| SEQ ID NO: 59 | HMG box primer 1 | 21 bases |
| SEQ ID NO: 60 | HMG box primer 2 | 26 bases |
| SEQ ID NO: 61 | neo R primer | 22 bases |
| SEQ ID NO: 62 | neo F primer | 22 bases |
| SEQ ID NO: 63 | Sox18 box A primer | 20 bases |
| SEQ ID NO: 64 | Sox18 box B primer | 20 bases |
| SEQ ID NO: 65 | neo R primer | 22 bases |
| SEQ ID NO: 66 | neo F primer | 22 bases |
| SEQ ID NO: 67 | Lacz A primer | 20 bases |
| SEQ ID NO: 68 | Lacz B primer | 20 bases |
| SEQ ID NO: 69 | Sox18 A primer | 20 bases |
| SEQ ID NO: 70 | Sox18 B primerG | 20 bases |
| SEQ ID NO: 71 | Sox18 box A primer | 20 bases |
| SEQ ID NO: 72 | Sox18 box B primer | 20 bases |
| SEQ ID NO: 73 | 5′ Sox18 A primer | 21 bases |
| SEQ ID NO: 74 | 5′ Sox18 B primer | 21 bases |
| SEQ ID NO: 75 | GAPDH F primer | 18 bases |
| SEQ ID NO: 76 | GAPDH R primer | 18 bases |
| SEQ ID NO: 77 | GMUQ450 primer | 18 bases |
| SEQ ID NO: 78 | GMUQ480 primer | 20 bases |
| SEQ ID NO: 79 | GMUQ238 primer | 28 bases |
| SEQ ID NO: 80 | GMUQ239 primer | 30 bases |
| SEQ ID NO: 81 | GMUQ529 primer | 23 bases |
| SEQ ID NO: 82 | GMUQ530 primer | 23 bases |
| SEQ ID NO: 83 | GMUQ401 primer | 36 bases |
| SEQ ID NO: 84 | GMUQ503 primer | 29 bases |
| SEQ ID NO: 85 | hSOX18 primer A | 17 bases |
| SEQ ID NO: 86 | hSOX18 primer B | 17 bases |
| SEQ ID NO: 87 | hSOX18 primer C | 17 bases |
| SEQ ID NO: 88 | hSOX18 primer D | 17 bases |
| SEQ ID NO: 89 | hSOX18 primer E | 17 bases |
| SEQ ID NO: 90 | hSOX18 primer F | 17 bases |
| SEQ ID NO: 91 | hSOX18 primer G | 17 bases |
| SEQ ID NO: 92 | hSOX18 primer H | 17 bases |
| SEQ ID NO: 93 | M13 forward primer | 17 bases |
| SEQ ID NO: 94 | M13 reverse primer | 17 bases |
| SEQ ID NO: 95 | SOX18 specific primer 1 | 24 bases |
| SEQ ID NO: 96 | SOX18 specific primer 2 | 24 bases |
| SEQ ID NO: 97 | Geysen library peptide | 8 residues |

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” is used herein to refer to polypeptides that vary by as much as 30%, preferably by as much as 20%, and more preferably by as much as 10% to the length of a reference polypeptide.

By “agent” is meant a naturally occurring or synthetically produced molecule which interacts either directly or indirectly with a target member, the level and/or functional activity of which are to be modulated.

“Amplification product” refers to a nucleic acid product generated by nucleic acid amplification techniques.

By “antigen-binding molecule” is meant a molecule that has binding affinity for a target antigen. It is understood that this term extends to immunoglobulins, immunoglobulin fragments and non-immunoglobulin derived protein frameworks that exhibit antigen-binding activity.

As used herein, the term “binds specifically” “specifically immuno-interactive” and the like refers to antigen-binding molecules that bind, or are otherwise immuno-interactive with, the polypeptide or polypeptide fragments of the invention but do not significantly bind to, or do not otherwise specifically immuno-interact with, homologous prior art polypeptides.

By “biologically active fragment” is meant a fragment of a full-length parent polypeptide which fragment retains the activity of the parent polypeptide. A biologically active fragment therefore modulates at least one activity selected from the group consisting of cell differentiation, vasculogenesis, angiogenesis, hair follicle development, cell proliferation and tumorigenesis, or elicit an immunogenic response to produce elements (e.g., antigen-binding molecules) that specifically bind to the parent polypeptide. As used herein, the term “biologically active fragment” includes deletion mutants and small peptides, for example of at least 8, preferably at least 10, more preferably at least 15, even more preferably at least 20 and even more preferably at least 30 contiguous amino acids, which comprise the above activities. . Peptides of this type may be obtained through the application of standard recombinant nucleic acid techniques or synthesised using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

The term “biological sample” as used herein refers to a sample that may be extracted, untreated, treated, diluted or concentrated from an animal. The biological sample may be selected from the group consisting of whole blood, serum, plasma, saliva, urine, sweat, ascitic fluid, peritoneal fluid, synovial fluid, amniotic fluid, cerebrospinal fluid, skin biopsy, and the like. Preferably, the biological sample is a tissue biopsy.

Throughout this specification, unless the context requires otherwise, the words “comprise”, “comprises” and “comprising” is understood to imply the inclusion of a stated step or element or group of steps or elements but not the exclusion of any other step or element or group of steps or elements.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions, or deletions that provide for functionally equivalent molecules. Accordingly, the term derivative encompasses molecules that have an activity selected from the group consisting of cell differentiation, vasculogenesis, angiogenesis, hair follicle development, cell proliferation, tumorigenesis, and the elicitation of an immunogenic response to produce elements (e.g., antigen-binding molecules) that specifically bind to the parent polypeptide.

“Homology” refers to the percentage number of amino acids that are identical or constitute conservative substitutions as defined in Table B infra. Homology may be determined using sequence comparison programs such as GAP (Deveraux et al. 1984). In this way, sequences of a similar or substantially different length to those cited herein might be compared by insertion of gaps into the alignment, such gaps being determined, for example, by the comparison algorithm used by GAP.

“Hybridisation” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that do not hybridise efficiently.

Reference herein to “immuno-interactive” includes reference to any interaction, reaction, or other form of association between molecules and in particular where one of the molecules is, or mimics, a component of the immune system.

By “immuno-interactivefragment” is meant a fragment of the polypeptide set forth in any one of SEQ ID NO: 2, 15 and 18 which fragment elicits an immune response, including the production of elements that specifically bind to said polypeptide, or variant or derivative thereof. As used herein, the term “immuno-interactivefragment” includes deletion mutants and small peptides, for example of at least six, preferably at least 8 and more preferably at least 12, even more preferably at least 15, even more preferably at least 18 and still even more preferably at least 20 contiguous amino acids, which comprise antigenic determinants or epitopes. Several such fragments may be joined together.

By “isolated” is meant material that is substantially or essentially free from components that normally accompany it in its native state. For example, an “isolated polynucleotide”, as used herein, refers to a polynucleotide, which has been purified from the sequences which flank it in a naturally occurring state, e.g., a DNA fragment which has been removed from the sequences which are normally adjacent to the fragment.

By “modulating” is meant increasing or decreasing, either directly or indirectly, the level and/or functional activity of a target molecule. For example, an agent may indirectly modulate the said level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

By “obtained from” is meant that a sample such as, for example, a nucleic acid extract or polypeptide extract is isolated from, or derived from, a particular source of the host. For example, the extract may be obtained from a tissue or a biological fluid isolated directly from the host.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it is understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

By “operably linked” is meant that transcriptional and translational regulatory nucleic acids are positioned relative to a polypeptide-encoding polynucleotide in such a manner that the polynucleotide is transcribed and the polypeptide is translated.

The term “patient” refers to patients of human or other mammal and includes any individual it is desired to examine or treat using the methods of the invention. However, it is understood that “patient” does not imply that symptoms are present. Suitable mammals that fall within the scope of the invention include, but are not restricted to, primates, livestock animals (e.g. sheep, cows, horses, donkeys, pigs), laboratory test animals (e.g. rabbits, mice, rats, guinea pigs, hamsters), companion animals (e.g. cats, dogs) and captive wild animals (e.g. foxes, deer, dingoes).

By “pharmaceutically-acceptable carrier” is meant a solid or liquid filler, diluent or encapsulating substance that may be safely used in topical or systemic administration.

The term “polynucleotide” or “nucleic acid” as used herein designates mRNA, RNA, cRNA, cDNA or DNA. The term typically refers to oligonucleotides greater than 30 nucleotides in length. Polynucleotide sequences are understood to encompass complementary strands as well as alternative backbones described herein.

The terms “polynucleotide variant” and “variant” refer to polynucleotides displaying substantial sequence identity with a reference polynucleotide sequence or polynucleotides that hybridise with a reference sequence under stringent conditions that are defined hereinafter. These terms also encompasses polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides in which one or more amino acids have been replaced by different amino acids. It is well understood in the art that some amino acids may be changed to others with broadly similar properties without changing the nature of the activity of the polypeptide (conservative substitutions) as described hereinafter. Accordingly, polypeptide variants as used herein encompass polypeptides that have an activity selected from the group consisting of cell differentiation, vasculogenesis, angiogenesis, hair follicle development, cell proliferation and tumorigenesis.

By “primer” is meant an oligonucleotide which, when paired with a strand of DNA, is capable of initiating the synthesis of a primer extension product in the presence of a suitable polymerising agent. The primer is preferably single-stranded for maximum efficiency in amplification but may alternatively be double-stranded. A primer must be sufficiently long to prime the synthesis of extension products in the presence of the polymerisation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotides, although it may contain fewer nucleotides. Primers can be large polynucleotides, such as from about 200 nucleotides to several kilobases or more. Primers may be selected to be “substantially complementary” to the sequence on the template to which it is designed to hybridise and serve as a site for the initiation of synthesis. By “substantially complementary”, it is meant that the primer is sufficiently complementary to hybridise with a target nucleotide sequence. Preferably, the primer contains no mismatches with the template to which it is designed to hybridise but this is not essential. For example, non-complementary nucleotides may be attached to the 5′ end of the primer, with the remainder of the primer sequence being complementary to the template. Alternatively, non-complementary nucleotides or a stretch of non-complementary nucleotides can be interspersed into a primer, provided that the primer sequence has sufficient complementarity with the sequence of the template to hybridise therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another nucleic acid, often called the “target nucleic acid”, through complementary base pairing. Probes may bind target nucleic acids lacking complete sequence complementarity with the probe, depending on the stringency of the hybridisation conditions. Probes can be labelled directly or indirectly.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” is understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridisation and washing procedures. The higher the stringency, the higher will be the degree of complementarity between immobilised target nucleotide sequences and the labelled probe polynucleotide sequences that remain hybridised to the target after washing.

“Stringent conditions” refers to temperature and ionic conditions under which only nucleotide sequences having a high frequency of complementary bases will hybridise. The stringency required is nucleotide sequence dependent and depends upon the various components present during hybridisation and subsequent washes, and the time allowed for these processes. Generally, in order to maximise the hybridisation rate, non-stringent hybridisation conditions are selected; about 20 to 25° C. lower than the thermal melting point (Tₘ). The Tₘ is the temperature at which 50% of specific target sequence hybridises to a perfectly complementary probe in solution at a defined ionic strength and pH. Generally, in order to require at least about 85% nucleotide complementarity of hybridised sequences, highly stringent washing conditions are selected to be about 5 to 15° C. lower than the Tₘ. In order to require at least about 70% nucleotide complementarity of hybridised sequences, moderately stringent washing conditions are selected to be about 15 to 30° C. lower than the Tₘ. Highly permissive (low stringency) washing conditions may be as low as 50° C. below the Tₘ, allowing a high level of mismatching between hybridised sequences. Those skilled in the art will recognise that other physical and chemical parameters in the hybridisation and wash stages can also be altered to affect the outcome of a detectable hybridisation signal from a specific level of homology between target and probe sequences. Other examples of stringency conditions are described in section 3.3.

By “therapeutically effective amount”, in the context of treating a condition selected from the group consisting of artherosclerosis, restenosis, pulmonary disease, and tissue injury, is meant the administration of that amount of SOX18 or modulatory agent that modulates the expression of Sox18 to an individual in need of such treatment, either in a single dose or as part of a series, that is effective for treatment of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a mini- chromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector typically depends on the compatibility of the vector with the host cell into which the vector is to be introduced. The vector may also include a selection marker such as an antibiotic resistance gene that can be used for selection of suitable transformants. Examples of such resistance genes are well known to those of skill in the art.

As used herein, underscoring or italicizing the name of a gene shall indicate the gene, in contrast to its protein product, which is indicated in the absence of any underscoring or italicizing. For example, “SOX18” shall mean the Sox18 gene or cDNA sequence, whereas “SOX18” shall indicate the protein product of the “Sox18” gene.

2. Isolated Polypeptides, Biologically Active Fragments, Polypeptide Variants and Derivatives

2.1 Polypeptides of the Invention

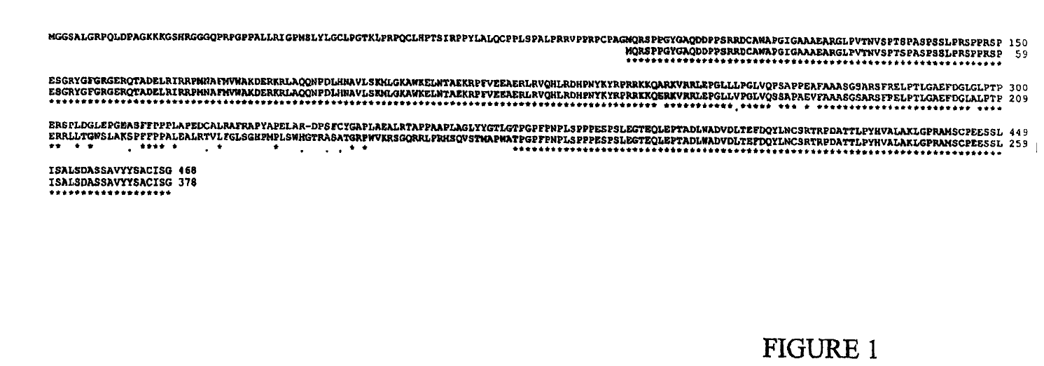

In work leading up to the present invention, the inventors discovered that the deduced polypeptide sequence of mouse Sox18 extends a further 91 residues upstream of that previously published (Dunn et al., 1995, W097/04090). Thus, the full-length mouse SOX18 polypeptide comprises 468 residues instead of 378 residues. Further sequencing of the mouse Sox18 genomic sequence also revealed a number of sequencing errors in the region encoding the SOX18 trans-activation domain. The revised deduced polypeptide sequence (SEQ ID NO: 2) has 65 amino acid substitutions and one deletion relative to the previously published sequence (see FIG. 1 for a sequence comparison). This corresponds to a percentage sequence identity between the sequences of about 83%.

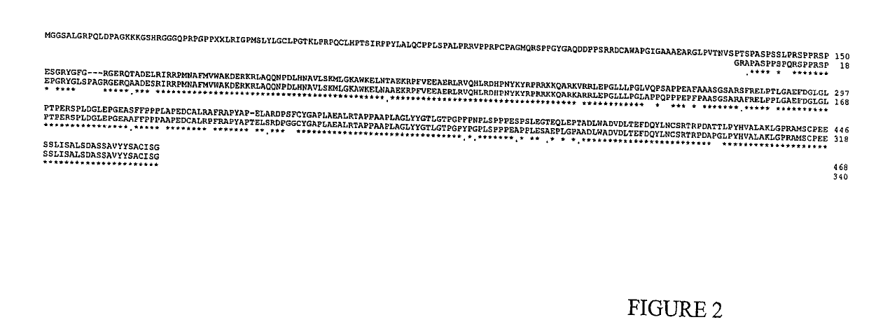

The deduced polypeptide sequence relating to human SOX18 has also been determined. In this respect, the present inventors surprisingly discovered that a sequence previously published as encoding the human HMG-box activating factor-2 (HAF-2; Stevens et al., 1996), a transcription factor that binds the human immunoglobulin heavy chain enhancer, in fact contains 33 sequence errors, which when rectified result in a nucleic acid sequence encoding a polypeptide (SEQ ID NO: 15) with less than 50% sequence identity to the published HAF-2 polypeptide sequence but displaying high sequence identity to murine SOX18 (about 88%; see FIG. 2 for a sequence comparison of murine and human SOX18 polypeptides). It was concluded that HAF-2 is human SOX18.

The full-length human SOX18 polypeptide sequence (SEQ ID NO: 18) was deduced subsequently from sequence analysis of a 1730 bp full-length Sox18 cDNA clone. The human sequence is shorter at the amino terminal end by 50 amino acids than the extended mouse Sox18 polypeptide sequence set forth in SEQ ID NO: 2.

2.2 Biologically Active Fragments

Biologically active fragments may be produced according to any suitable procedure known in the art. For example, a suitable method may include first producing a fragment of said isolated polypeptide and then testing the fragment for the appropriate biological activity. In one embodiment, biological activity of the fragment may be tested by contacting a MEF2C protein with a fragment of the polypeptide, and detecting the presence of a complex comprising the MEF2C and the fragment, which indicates that said fragment is a biologically active fragment.

Any suitable technique for determining formation of the complex may be used. For example, the complex may be detected by Western blotting, precipitation techniques inclusive of “GST pull-down ” assays and immuno-precipitation, surface plasmon resonance (e.g., BIAcore™ system from Pharmacia Biosensor and chromatographic techniques which are well known those of skill in the art.

In another embodiment, biological activity of the fragment is tested by introducing a fragment of the polypeptide or a polynucleotide from which the fragment can be translated into a cell, and detecting an activity selected from the group consisting of cell differentiation, vasculogenesis, angiogenesis and hair follicle development, which is indicative of said fragment being a said biologically active fragment. In a preferred embodiment, the cell differentiation is endothelial cell differentiation.

The biological fragment preferably comprises one or more of a SOX18 C terminal domain (CCT), a SOX18 HMG box domain and a SOX18 trans-activation domain. In one embodiment, the biologically active fragment comprises a CCT domain of SOX18. In a preferred embodiment of this type, the biologically active fragment comprises a contiguous sequence of amino acids contained within SEQ ID NO: 30 at least 8 amino acids in length. In a preferred embodiment of this type, the biologically active fragment comprises the sequence set forth in SEQ ID NO: 30. In an alternate embodiment of this type, the biologically active fragment comprises the sequence set forth in SEQ ID NO: 13 or 7 In yet another embodiment of this type, the biologically active fragment comprises the sequence set forth in SEQ ID NO: 28 or 29.

Preferably, the biologically active fragment, in addition to the CCT domain, further comprises a domain selected from the group consisting of the SOX18 HMG box domain and the SOX18 trans-activation domain, or variant of these. In a preferred embodiment of this type, the HMG box domain comprises the sequence set forth in SEQ ID NO: 9 or 23 and the trans-activation domain comprises the sequence set forth in SEQ ID NO: 11 or 25.

The invention also extends to biological fragments of the above polypeptides, which can elicit an immune response in an animal and preferably in a heterologous animal from which the polypeptide is obtained. For example exemplary polypeptide fragments of 8 residues in length, which could elicit an immune response, include but are not limited to residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 233-240, 241-248, 249-256, 257-264, 265-272, 273-280, 281-288, 289-296, 297-304, 305-312, 313-320, 321-328, 329-336, 337-344, 345-352, 353-360, 361-368, 369-376, 377-384, 385-392, 393-400, 401-408, 409-416, 417-424, 425-432, 433-440, 441-448, 449-456, 457-464, 261-468 of SEQ ID NO: 18. In an alternate embodiment of this type, the biologically active fragment is selected from residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 233-240, 241-248, 249-256, 257-264, 265-272, 273-280, 281-288, 289-296, 297-304, 305-312, 313-320, 321-328, 329-336, 333-340 of SEQ ID NO: 15. In another embodiment of this type, the biologically active fragment is selected from residues 1-8, 9-16, 17-24, 25-32, 33-40, 41-48, 49-56, 57-64, 65-72, 73-80, 81-88, 84-91 of SEQ ID NO: 7.

2.3 Polypeptide Variants

The invention also contemplates polypeptide variants of the polypeptides of the invention wherein said variants promote at least one activity selected from the group consisting of cell differentiation, vasculogenesis, angiogenesis and hair follicle development. Suitable methods of producing polypeptide variants include, for example, replacing at least one amino acid of a parent polypeptide comprising the sequence set forth in any one of SEQ ID NO: 2 15 and 18, or a biologically active fragment thereof, with a different amino acid to produce a modified polypeptide. The modified polypeptide is then tested by contacting that polypeptide with a MEF2C protein and detecting the presence of a complex comprising the MEF2C and the modified polypeptide, which indicates that said fragment is a polypeptide variant.

In another embodiment, a polypeptide variant is produced by replacing at least one amino acid of a parent polypeptide comprising the sequence set forth in any one of SEQ ID NO: 2, 15 and 18, or a biologically active fragment thereof, with a different amino acid to produce a modified polypeptide, introducing said polypeptide or a polynucleotide from which the fragment can be translated into a cell, and detecting at least one activity selected from the group consisting of cell differentiation, vasculogenesis, angiogenesis and hair follicle development, which indicates that the modified polypeptide is a polypeptide variant.

Suitable assays for assaying the above activities are known to persons of skill in the art. Examples of assays that may be used in accordance with the present invention are described in Section 6.

In general, variants have at least 85% homology, more preferably at least 90% homology, preferably at least 95% homology, and more preferably at least 97% homology to a polypeptide as for example shown in SEQ ID NO: 2, 15 or 18, or fragments thereof. It is preferred that variants display at least 85% identity, more preferably at least 90% identity, preferably at least 95% identity, and more preferably at least 98% identity with a polypeptide as for example shown in SEQ ID NO: 2, 15 or 18, or fragments thereof. In this respect, the window of comparison spans about the full length of the polypeptide.

Suitable variants can be obtained from any suitable animal. For example, the variant may comprise the sequence set forth in SEQ ID NO: 32, corresponding to chicken Sox18. Preferably, the variants are obtained from a mammal as for example described in Section 3.2 infra.

2.4 Methods of Producing Polypeptide Variants

2.4.1 Mutagenesis

Polypeptide variants according to the invention can be identified either rationally, or via established methods of mutagenesis (see, for example, Watson, J. D. et al., “MOLECULAR BIOLOGY OF THE GENE”, Fourth Edition, Benjamin/Cummings, Menlo Park, Calif., 1987). Significantly, a random mutagenesis approach requires no a priori information about the gene sequence that is to be mutated. This approach has the advantage that it assesses the desirability of a particular mutant based on its function, and thus does not require an understanding of how or why the resultant mutant protein has adopted a particular conformation. Indeed, the random mutation of target gene sequences has been one approach used to obtain mutant proteins having desired characteristics (Leatherbarrow, R. 1986; Knowles, J. R., 1987; Shaw, W. V., 1987; Gerit, J. A. 1987). Alternatively, where aparticular sequence alteration is desired, methods of site-directed mutagenesis can be employed. Thus, such methods may be used to selectively alter only those amino acids of the protein that are believed to be important (Craik, C. S., 1985; Cronin, et al., 1988; Wilks, et al., 1988).

Variant peptides or polypeptides, resulting from rational or established methods of mutagenesis or from combinatorial chemistries as hereinafter described, may comprise conservative amino acid substitutions. Exemplary conservative substitutions in an polypeptide or polypeptide fragment according to the invention may be made according to the following table:

TABLE B

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Substantial changes in function are made by selecting substitutions that are less conservative than those shown in TABLE B. Other replacements would be non-conservative substitutions and relatively fewer of these may be tolerated. Generally, the substitutions which are likely to produce the greatest changes in a polypeptide's properties are those in which (a) a hydrophilic residue (e.g., Ser or Asn) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a smaller side chain (e.g., Ala, Ser) or no side chain (e.g., Gly) is substituted for, or by, one having a bulky side chain (e.g., Phe or Trp).

What constitutes suitable variants may be determined by conventional techniques. For example, nucleic acids encoding a polypeptide according to SEQ ID NO: 2, 15 or 18 can be mutated using either random mutagenesis for example using transposon mutagenesis, or site-directed mutagenesis as described, for example, in Section 3.2 infra.

2.4.2 Peptide Libraries Produced by Combinatorial Chemistry

A number of facile combinatorial technologies can be utilised to synthesise molecular libraries of immense diversity. In the present case, variants of a polypeptide, or preferably a polypeptide fragment according to the invention, can be synthesised using such technologies. These polypeptide fragments may be immuno-interactive or may bind to a binding partner involved in an activity selected from the group consisting of cell differentiation, vasculogenesis, angiogenesis, hair follicle development. The specific binding partner is preferably MEF2C. Variants can be screened subsequently using the methods described in Sections 2.3 and 7.

In one embodiment, soluble synthetic peptide combinatorial libraries (SPCLs) are produced which offer the advantage of working with free peptides in solution, thus permitting adjustment of peptide concentration to accommodate a particular assay system. SPCLs are preferably prepared as hexamers. In this regard, a majority of binding sites is known to involve four to six residues. Cysteine is preferably excluded from the mixture positions to avoid the formation of disulphides and more difficult-to-define polymers. Exemplary methods of producing SPCLs are disclosed by Houghten et al. 1991 and 1992, Appel et al. 1992, and Pinilla et al 1992 and 1993.

Preparation of combinatorial synthetic peptide libraries may employ either t-butyloxycarbonyl (t-Boc) or 9-fluorenylmethyloxycarbonyl (Fmoc) chemistries (see Chapter 9.1, of Coligan et al, supra; Stewart and Young, 1984, Solid Phase Peptide Synthesis, 2nd ed. Pierce Chemical Co., Rockford, Ill.; and Atherton and Sheppard, 1989, Solid Phase Peptide Synthesis: A Practical Approach. IRL Press, Oxford) preferably, but not exclusively, using one of two different approaches. The first of these approaches, preferably termed the “split-process-recombine” or “split synthesis” method, was described first by Furka et al. 1988 and 1991 and Lam et al. 1991, and reviewed later by Eichler et al. 1995 and Balkenhohl et al. 1996. Briefly, the split synthesis method involves dividing a plurality of solid supports such as polymer beads into n equal fractions representative of the number of available amino acids for each step of the synthesis (e.g., 20 L-amino acids), coupling a single respective amino acid to each polymer bead of a corresponding fraction, and then thoroughly mixing the polymer beads of all the fractions together. This process is repeated for a total of x cycles to produce a stochastic collection of up to N^{x} different compounds. The peptide library so produced may be screened for example with a suitably labelled antigen-binding molecule or any other binding partner (e.g., MEF2C, as hereinafter described) that binds specifically to a polypeptide according to any one of SEQ ID NO: 2, 15 or 18. Upon detection, some of the positive beads are selected for sequencing to identify the active peptide. Such peptide may be subsequently cleaved from the beads, and assayed using the same antigen-binding molecule or other binding partner to identify the most active peptide sequence.

The second approach, the chemical ratio method, prepares mixed peptide resins using a specific ratio of amino acids empirically defined to give equimolar incorporation of each amino acid at each coupling step. Each resin bead contains a mixture of peptides. Approximate equimolar representation can be confirmed by amino acid analysis (Dooley and Houghten, 1993; Eichler and Houghten, 1993). Preferably, the synthetic peptide library is produced on polyethylene rods, or pins, as a solid support, as for example disclosed by Geysen et al. (1986). An exemplary peptide library of this type may consist of octapeptides in which the third and fourth positions represent defined amino acids selected from natural and non-natural amino acids, and in which the remaining six positions represent a randomized mixture of amino acids. This peptide library can be represented by the formula Ac-X₁X₂O₁O₂X₃X₄X₅X₆-S_{S}[SEQ ID NO: 97], where O₁ and O₂ are each defined amino acids, X₁₋₆ are a randomized mixture of amino acids and S, is the solid support. Peptide mixtures remain on the pins when assayed against a soluble receptor molecule. For example, the peptide library of Geysen (1986, Immun. Today 6: 364-369; and Geysen et al., Ibid), comprising for example dipeptides, is first screened for the ability to bind to a target molecule. The most active dipeptides are then selected for an additional round of testing comprising linking, to the starting dipeptide, an additional residue (or by internally modifying the components of the original starting dipeptide) and then screening this set of candidates for the desired activity. The best tripeptide is used as the basis of a tetrapeptide and so on until an optimized sequence up to an octapeptide with the desired properties has been determined.

2.4.3 Alanine Scanning Mutagenesis

In one embodiment, the invention herein utilises a systematic analysis of a polypeptide or polypeptide fragment according to the invention to determine the residues in the polypeptide or fragment that are involved in the interaction with a specific binding partner such as, for example, MEF2C. Such analysis is conveniently performed using recombinant DNA technology. In general, a DNA sequence encoding the polypeptide or fragment is cloned and manipulated so that it may be expressed in a convenient host. DNA encoding the polypeptide or fragment can be obtained from a genomic library, from cDNA derived from mRNA in cells expressing the said polypeptide or fragment, or by synthetically constructing the DNA sequence (Sambrook et al., supra; Ausubel et al., supra).

The wild-type DNA encoding the polypeptide or fragment is then inserted into an appropriate plasmid or vector as described herein. In particular, prokaryotes are preferred for cloning and expressing DNA sequences to produce variants of the polypeptide or fragment. For example, E. coli K12 strain 294 (ATCC No. 31446) may be used, as well as E. coli B, E. coli X1776 (ATCC No. 31537), and E. coli c600 and c600hfl, and E. coli W3110 (F⁻, γ⁻, prototrophic, ATCC No. 27325), bacilli such as Bacillus subtilis, and other enterobacteriaceae such as Salmonella typhimurium or Serratia marcescens, and various Pseudomonas species. A preferred prokaryote is E. coli W3110 (ATCC 27325).

Once the polypeptide or fragment is cloned, site-specific mutagenesis as for example described by Carter et al. (1986) or by Zoller et al (1987), cassette mutagenesis as for example described by Wells et al. (1985), restriction selection mutagenesis as for example described by Wells et al. (1986), or other known techniques may be performed on the cloned DNA to produce the variant DNA that encodes for the changes in amino acid sequence defined by the residues being substituted. When operably linked to an appropriate expression vector, variants are obtained. In some cases, recovery of the variant may be facilitated by expressing and secreting such molecules from the expression host by use of an appropriate signal sequence operably linked to the DNA sequence encoding the variant. Such methods are well known to those skilled in the art. Of course, other methods may be employed to produce such polypeptides or fragments such as the in vitro chemical synthesis of the desired polypeptide variant (Barany et al. In The Peptides, eds. E. Gross and J. Meienhofer (Academic Press: N.Y. 1979), Vol. 2, pp. 3-254).

Once the different variants are produced, they are contacted with a SOX18-specific binding partner (e.g., MEF2C) and the interaction, if any, between this binding partner and each variant is determined. These activities are compared to the activity of the parent polypeptide or fragment with the same binding partner molecule to determine which of the amino acid residues in the active domain are involved in the interaction with the binding partner. The scanning amino acid used in such an analysis may be any different amino acid from that substituted, i.e., any of the 19 other naturally occurring amino acids.

The interaction between the SOX18-specific binding partner, and parent and variant, respectively, can be measured by any convenient assay as for example described herein. While any number of analytical measurements may be used to compare activities, a convenient one for binding of the SOX18-specific binding partner is the dissociation constant K_{d} of the complex formed between the variant and that binding partner as compared to the K_{d} for the parent polypeptide or fragment. Generally, a two-fold increase or decrease in K_{d} per analogous residue substituted by the substitution may suggest that the substituted residue(s) is active in the interaction of the parent polypeptide or fragment with the target binding partner.

When a suspected or known active amino acid residue is subjected to scanning amino acid analysis, the amino acid residues immediately adjacent thereto should be scanned. Three residue-substituted polypeptides can be made. One contains a scanning amino acid, preferably alanine, at position N that is the suspected or known active amino acid. The two others contain the scanning amino acid at position N+1 and N−1. If each substituted polypeptide or fragment causes a greater than about two-fold effect on K_{d} for the receptor, the scanning amino acid is substituted at position N+2 and N-2. This is repeated until at least one, and preferably four, residues are identified in each direction which have less than about a two-fold effect on K_{d} or either of the ends of the parent polypeptide or fragment are reached. In this manner, one or more amino acids along a continuous amino acid sequence that are involved in the interaction with the particular binding partner molecule can be identified.

The active amino acid residue identified by amino acid scan is typically one that contacts the SOX18-specific binding partner directly. However, active amino acids may also indirectly contact the binding partner through salt bridges formed with other residues or small molecules such as H₂O or ionic species such as Na⁺, Ca⁺², Mg⁺², or Zn⁺².

In some cases, the substitution of a scanning amino acid at one or more residues results in a residue-substituted polypeptide which is not expressed at levels that allow for the isolation of quantities sufficient to carry out analysis of its activity with the SOX18-specific binding partner. In such cases, a different scanning amino acid, preferably an isosteric amino acid, can be used.

Among the preferred scanning amino acids are relatively small, neutral amino acids. Such amino acids include alanine, glycine and serine. Alanine is the preferred scanning amino acid among this group because it eliminates the side-chain beyond the beta-carbon and is less likely to alter the main-chain conformation of the variant. Alanine is also preferred because it is the most common amino acid. Further, it is frequently found in both buried and exposed positions (Creighton, The Proteins, W. H. Freeman & Co., N.Y.; Chothia, 1976). If alanine substitution does not yield adequate amounts of variant, an isosteric amino acid can be used. Alternatively, the following amino acids in decreasing order of preference may be used: Ser, Asn, and Leu.

Once the active amino acid residues are identified, isosteric amino acids may be substituted. Such isosteric substitutions need not occur in all instances and may be performed before any active amino acid is identified. Such isosteric amino acid substitution is performed to minimise the potential disruptive effects on conformation that some substitutions can cause. Isosteric amino acids are shown in the table below:

TABLE C

| Polypeptide Amino Acid | Isosteric Scanning Amino Acid |
|---|---|
| Ala (A) | Ser, Gly |
| Glu (E) | Gln, Asp |
| Gln (Q) | Asn, Glu |
| Asp (D) | Asn, Glu |
| Asn (N) | Ala, Asp |
| Leu (L) | Met, Ile |
| Gly (G) | Pro, Ala |
| Lys (K) | Met, Arg |
| Ser (S) | Thr, Ala |
| Val (V) | Ile, Thr |
| Arg (R) | Lys, Met, Asn |
| Thr (T) | Ser, Val |
| Pro (P) | Gly |
| Ile (I) | Met, Leu, Val |
| Met (M) | Ile, Leu |
| Phe (F) | Tyr |
| Tyr (Y) | Phe |
| Cys (C) | Ser, Ala |
| Trp (W) | Phe |
| His (H) | Asn, Gln |

The method herein can be used to detect active amino acid residues within different domains of a polypeptide or fragment according to the invention. Once this identification is made various modifications to the parent polypeptide or fragment may be made to modify the interaction between the parent polypeptide or fragment and a specific binding partner.

2.4.4 Polypeptide or Peptide Libraries Produced by Phage Display

The identification of variants can also be facilitated through the use of a phage (or phagemid) display protein ligand screening system as for example described by Lowman, et al. (1991), Markland, et al. (1991,), Roberts, et al. (1992), Smith, G. P. (1985), Smith, et al. (1990) and Lardner et al. (U.S. Pat. No. 5,223,409). In general, this method involves expressing a fusion protein in which the desired protein ligand is fused to the N-terminus of a viral coat protein (such as the M13 Gene III coat protein, or a lambda coat protein).

In one embodiment, a library of phage is engineered to display novel peptides within the phage coat protein sequences. Novel peptide sequences are generated by random mutagenesis of gene fragments encoding a polypeptide of the invention or biologically active fragment using error-prone PCR, or by in vivo mutation by E. coli mutator cells. The novel peptides displayed on the surface of the phage are placed in contact, with a SOX18-specific binding partner molecule (e.g., MEF2C, an antigen-binding molecule). Phage that display coat protein having peptides that are capable of binding to a binding partner are immobilised by such treatment, whereas all other phage can be washed away. After the removal of unbound phage, the bound phage can be amplified, and the DNA encoding their coat proteins can be sequenced. In this manner, the amino acid sequence of the embedded peptide or polypeptide can be deduced.

In more detail, the method involves (a) constructing a replicable expression vector comprising a first gene encoding a polypeptide or fragment of the invention, a second gene encoding at least a portion of a natural or wild-type phage coat protein wherein the first and second genes are heterologous, and a transcription regulatory element operably linked to the first and second genes, thereby forming a gene fusion encoding a fusion protein; (b) mutating the vector at one or more selected positions within the first gene thereby forming a family of related plasmids; (c) transforming suitable host cells with the plasmids; (d) infecting the transformed host cells with a helper phage having a gene encoding the phage coat protein; (e) culturing the transformed infected host cells under conditions suitable for forming recombinant phagemid particles containing at least a portion of the plasmid and capable of transforming the host, the conditions adjusted so that no more than a minor amount of phagemid particles display more than one copy of the fusion protein on the surface of the particle; (f) contacting the phagemid particles with a SOX18-specific binding partner that binds to the parent polypeptide or fragment so that at least a portion of the phagemid particles bind to the binding partner; and (g) separating the phagemid particles that bind from those that do not. Preferably, the method further comprises transforming suitable host cells with recombinant phagemid particles that bind to the SOX18-specific binding partner and repeating steps (d) through (g) one or more times.

Preferably in this method the plasmid is under tight control of the transcription regulatory element, and the culturing conditions are adjusted so that the amount or number of phagemid particles displaying more than one copy of the fusion protein on the surface of the particle is less than about 1%. Also, preferably, the amount of phagemid particles displaying more than one copy of the fusion protein is less than 10% of the amount of phagemid particles displaying a single copy of the fusion protein. Most preferably, the amount is less than 20%.

Typically in this method, the expression vector could further contain a secretory signal sequence fused to the DNA encoding each subunit of the polypeptide and the transcription regulatory element is a promoter system. Preferred promoter systems are selected from lac Z, λ_{PL}, tac, T7 polymerase, tryptophan, and alkaline phosphatase promoters and combinations thereof. Also, normally the method employs a helper phage selected from M13K07, M13R408, M13-VCS, and Phi X 174. The preferred helper phage is M13K07, and the preferred coat protein is the M13 Phage gene III coat protein. The preferred host is E. coli, and protease-deficient strains of E. coli.

Repeated cycles of variant selection are used to select for higher and higher affinity binding by the phagemid selection of multiple amino acid changes that are selected by multiple selection cycles. Following a first round of phagemid selection, involving a first region or selection of amino acids in the ligand polypeptide, additional rounds of phagemid selection in other regions or amino acids of the ligand polypeptide are conducted. The cycles of phagemid selection are repeated until the desired affinity properties of the ligand polypeptide are achieved.

It will be appreciated that the amino acid residues that form the binding domain of the polypeptide or fragment may not be sequentially linked and may reside on different subunits of the polypeptide or fragment. That is, the binding domain tracks with the particular secondary structure at the binding site and not the primary structure. Thus, generally, mutations are introduced into codons related to amino acids within a particular secondary structure at sites directed away from the interior of the polypeptide so that they will have the potential to interact with the SOX18-specific binding partner.

The phagemid-display method herein contemplates fusing a polynucleotide encoding the polypeptide or fragment (polynucleotide 1) to a second polynucleotide (polynucleotide 2) such that a fusion protein is generated during transcription. Polynucleotide 2 is typically a coat protein gene of a phage, and preferably it is the phage M13 gene III coat protein, or a fragment thereof. Fusion of polynucleotides 1 and 2 may be accomplished by inserting polynucleotide 2 into a particular site on a plasmid that contains polynucleotide 1, or by inserting polynucleotide 1 into a particular site on a plasmid that contains polynucleotide 2.

Between polynucleotide 1 and polynucleotide 2, DNA encoding a termination codon may be inserted, such termination codons being UAG (amber), UAA (ocher), and UGA (opel) (see for example, Davis et al., Microbiology (Harper and Row: New York, 1980), pages 237, 245-247, and 274). The termination codon expressed in a wild-type host cell results in the synthesis of the polynucleotide 1 protein product without the polynucleotide 2 protein attached. However, growth in a suppressor host cell results in the synthesis of detectable quantities of fused protein. Such suppressor host cells contain a tRNA modified to insert an amino acid in the termination codon position of the mRNA, thereby resulting in production of detectable amounts of the fusion protein. Suppressor host cells of this type are well known and described, such as E. coli suppressor strain (Bullock et al., 1987). Any acceptable method may be used to place such a termination codon into the mRNA encoding the fusion polypeptide.

The suppressible codon may be inserted between the polynucleotide encoding the polypeptide or fragment and a second polynucleotide encoding at least a portion of a phage coat protein. Alternatively, the suppressible termination codon may be inserted adjacent to the fusion site by replacing the last amino acid triplet in the polypeptide/fragment or the first amino acid in the phage coat protein. When the phagemid containing the suppressible codon is grown in a suppressor host cell, it results in the detectable production of a fusion polypeptide containing the polypeptide or fragment and the coat protein. When the phagemid is grown in a non-suppressor host cell the polypeptide or fragment is synthesised substantially without fusion to the phage coat protein due to termination at the inserted suppressible triplet encoding UAG, UAA, or UGA. In the non-suppressor cell the polypeptide is synthesised and secreted from the host cell due to the absence of the fused phage coat protein which otherwise anchored it to the host cell.

The polypeptide or fragment may be altered at one or more selected codons. An alteration is defined as a substitution, deletion, or insertion of one or more codons in the gene encoding the polypeptide or fragment that results in a change in the amino acid sequence as compared with the unaltered or native sequence of the said polypeptide or fragment. Preferably, the alterations are made by substitution of at least one amino acid with any other amino acid in one or more regions of the molecule. The alterations may be produced by a variety of methods known in the art, as for example described in Section 2.3. These methods include, but are not limited to, oligonucleotide-mediated mutagenesis and cassette mutagenesis as described for example herein.

For preparing the SOX18-specific binding partner molecule and binding it with the phagemid, the binding partner molecule is attached to a suitable matrix such as agarose beads, acrylamide beads, glass beads, cellulose, various acrylic copolymers, hydroxyalkyl methacrylate gels, polyacrylic acid, polymethacrylic copolymers, nylon, neutral and ionic carriers, and the like. Attachment of the binding partner molecule to the matrix may be accomplished by methods described in Methods Enzymol., 44: (1976), or by other means known in the art.

After attachment of the specific binding partner molecule to the matrix, the immobilised binding partner is contacted with the library of phagemid particles under conditions suitable for binding of at least a portion of the phagemid particles with the immobilised binding partner or target. Normally, the conditions, including pH, ionic strength, temperature, and the like mimic physiological conditions.

Bound phagemid particles (“binders”) having high affinity for the immobilised target are separated from those having a low affinity (and thus do not bind to the target) by washing. Binders may be dissociated from the immobilised target by a variety of methods. These methods include competitive dissociation using the wild-type ligand, altering pH and/or ionic strength, and methods known in the art.

Suitable host cells are infected with the binders and helper phage, and the host cells are cultured under conditions suitable for amplification of the phagemid particles. The phagemid particles are then collected and the selection process is repeated one or more times until binders having the desired affinity for the target molecule are selected.

2.4.5 Rational Drug Design

Variants of an isolated polypeptide according to the invention or a biologically active fragment thereof may also be obtained using the principles of conventional or of rational drug design as for example described by Andrews, et al. (1990), McPherson, A. (1990), Hol,. et al. (1989), Hol, W. G. J. (1989), Hol, W. G. J. (1986).

In accordance with the methods of conventional drug design, the desired variant molecules are obtained by randomly testing molecules whose structures have an attribute in common with the structure of a parent polypeptide or biologically active fragment according to the invention. The quantitative contribution that results from a change in a particular group of a binding molecule can be determined by measuring the capacity of competition or cooperativity between the parent polypeptide or polypeptide fragment and the candidate polypeptide variant.

In one embodiment of rational drug design, the polypeptide variant is designed to share an attribute of the most stable three-dimensional conformation of a polypeptide or polypeptide fragment according to the invention. Thus, the variant may be designed to possess chemical groups that are oriented in a way sufficient to cause ionic, hydrophobic, or van der Waals interactions that are similar to those exhibited by the polypeptide or polypeptide fragment of the invention. In a second method of rational design, the capacity of a particular polypeptide or polypeptide fragment to undergo conformational “breathing” is exploited. Such “breathing”—the transient and reversible assumption of a different molecular conformation—is a well-appreciated phenomenon, and results from temperature, thermodynamic factors, and from the catalytic activity of the molecule. Knowledge of the 3-dimensional structure of the polypeptide or polypeptide fragment facilitates such an evaluation. An evaluation of the natural conformational changes of a polypeptide or polypeptide fragment facilitates the recognition of potential hinge sites, potential sites at which hydrogen bonding, ionic bonds or van der Waals bonds might form or might be eliminated due to the breathing of the molecule, etc. Such recognition permits the identification of the additional conformations that the polypeptide or polypeptide fragment could assume, and enables the rational design and production of mimetic polypeptide variants that share such conformations.

The preferred method for performing rational mimetic design employs a computer system capable of forming a representation of the three-dimensional structure of the polypeptide or polypeptide fragment (such as those obtained using RIBBON (Priestle, J., 1988, J. Mol. Graphics 21: 572), QUANTA (Polygen), INSIGHT 11 (MSI), or Nanovision (American Chemical Society)). Such analyses are exemplified by Hol, et al. (In: “MOLECULAR RECOGNITION: CHEMICAL AND BIOCHEMICAL PROBLEMS”, supra, Hol, W. G. J. (1989, supra) and Hol, W. G. J., (1986, supra).

In lieu of such direct comparative evaluations of candidate polypeptide variants, screening assays may be used to identify such molecules. Such assays preferably exploit the capacity of the variant to bind to a SOX18-specific binding partner such as MEF2C or an antigen-binding molecule specific to a SOX18 polypeptide of the invention and/or to promote an activity selected from the group consisting of cell differentiation, vasculogenesis, angiogenesis and/or hair follicle development.

2.5 Polypeptide Derivatives

With reference to suitable derivatives of the invention, such derivatives include amino acid deletions and/or additions to a polypeptide, fragment or variant of the invention, wherein said derivatives bind a SOX18-specific binding partner or promote at least one activity selected from the group consisting of cell differentiation, vasculogenesis, angiogenesis and/or hair follicle development. “Additions” of amino acids may include fusion of the polypeptides, fragments and polypeptide variants of the invention with other polypeptides or proteins. For example, it will be appreciated that said polypeptides, fragments or variants may be incorporated into larger polypeptides, and that such larger polypeptides may also be expected to bind a SOX18-specific binding partner or to promote an activity as mentioned above.

The polypeptides, fragments or variants of the invention may be fused to a further protein, for example, which is not derived from the original host. The further protein may assist in the purification of the fusion protein. For instance, a polyhistidine tag or a maltose binding protein may be used in this respect as described in more detail below. Other possible fusion proteins are those which produce an immunomodulatory response. Particular examples of such proteins include Protein A or glutathione S-transferase (GST).

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptides, fragments and variants of the invention.

Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2, 4, 6-trinitrobenzene sulphonic acid (TNBS).

The carboxyl group may be modified by carbodiimide activation via O-acylisourea formation followed by subsequent derivitisation, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, omithine, sarcosine, 2-thienyl alanine and/or D-isomers of amino acids. A list of unnatural amino acids contemplated by the present invention is shown in TABLE D.

TABLE D

| Non-conventional amino acid | Non-conventional amino acid |
|---|---|
| α-aminobutyric acid | L-N-methylalanine |
| α-amino-α-methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-medlylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-α-methylalanine | α-methylcyclohexylalanine |
| D-α-methylarginine | α-methylcylcopentylalanine |
| D-α-methylasparagine | α-methyl-α-napthylalanine |
| D-α-methylaspartate | α-methylpenicillamine |
| D-α-methylcysteine | N-(4-aminobutyl)glycine |
| D-α-methylglutamine | N-(2-aminoethyl)glycine |
| D-α-methylhistidine | N-(3-aminopropyl)glycine |
| D-α-methylisoleucine | N-amino-α-methylbutyrate |
| D-α-methylleucine | α-napthylalanine |
| D-α-methyllysine | N-benzylglycine |
| D-α-methylmethionine | N-(2-carbamylediyl)glycine |
| D-α-methylornithiine | N-(carbamylmethyl)glycine |
| D-α-methylphenylalanine | N-(2-carboxyethyl)glycine |
| D-α-methylproline | N-(carboxymethyl)glycine |
| D-α-methylserine | N-cyclobutylglycine |
| D-α-methylthreonine | N-cycloheptylglycine |
| D-α-methyltryptophan | N-cyclohexylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-(2,2-diphenylethyl | N-(N-(3,3-diphenylpropyl |
| carbamylmethyl)glycine | carbamylmethyl)glycine |
| 1-carboxy-1-(2,2-diphenyl-ethyl | |
| amino)cyclopropane | |

Also contemplated is the use of crosslinkers, for example, to stabilise 3D conformations of the polypeptides, fragments or variants of the invention, using homo-bifunctional cross linkers such as bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety or carbodiimide. In addition, peptides can be conformationally constrained, for example, by introduction of double bonds between C_{α} and C_{β} atoms of amino acids, by incorporation of C_{α} and N_{α}-methylamino acids, and by formation of cyclic peptides or analogues by introducing covalent bonds such as forming an amide bond between the N and C termini between two side chains or between a side chain and the N or C terminus of the peptides or analogues. For example, reference may be made to: Marlowe (1993) who describes peptide cyclisation on TFA resin using trimethylsilyl (TMSE) ester as an orthogonal protecting group; Pallin and Tam (1995) who describe the cyclisation of unprotected peptides in aqueous solution by oxime formation; Algin et al (1994) who disclose solid-phase synthesis of head-to-tail cyclic peptides via lysine side-chain anchoring; Kates et al (1993) who describe the production of head-to-tail cyclic peptides by three-dimensional solid phase strategy; Tumelty et al (1994) who describe the synthesis of cyclic peptides from an immobilised activated intermediate, wherein activation of the immobilised peptide is carried out with N-protecting group intact and subsequent removal leading to cyclisation; McMurray et al (1994) who disclose head-to-tail cyclisation of peptides attached to insoluble supports by means of the side chains of aspartic and glutamic acid; Hruby et al (1994) who teach an alternate method for cyclising peptides via solid supports; and Schmidt and Langer (1997) who disclose a method for synthesising cyclotetrapeptides and cyclopentapeptides. The foregoing methods may be used to produce conformationally constrained polypeptides that bind to a SOX18-specific binding partner or promote an activity selected from the group consisting of cell differentiation, vasculogenesis, angiogenesis and/or hair follicle development.

The invention also contemplates polypeptides, fragments or variants of the invention that have been modified using ordinary molecular biological techniques so as to improve their resistance to proteolytic degradation or to optimise solubility properties or to render them more suitable as an immunogenic agent.

2.6 Methods of Preparing the Polypeptides of the Invention

Polypeptides of the inventions may be prepared by any suitable procedure known to those of skill in the art. For example, the polypeptides may be prepared by a procedure including the steps of:

(a) preparing a recombinant polynucleotide comprising a nucleotide sequence encoding a polypeptide comprising the sequence set forth in any one of SEQ ID NO: 2, 15 or 18, or variant or derivative of these, which nucleotide sequence is operably linked to transcriptional and translational regulatory nucleic acid;

(b) introducing the recombinant polynucleotide into a suitable host cell;

(c) culturing the host cell to express recombinant polypeptide from said recombinant polynucleotide; and

(d) isolating the recombinant polypeptide.

Preferably, said nucleotide sequence comprises the sequence set forth in any one of SEQ ID NO: 1, 3, 5, 14, 16, 17, 19 and 21.

The recombinant polynucleotide preferably comprises either an expression vector that may be a self-replicating extra-chromosomal vector such as a plasmid, or a vector that integrates into a host genome.

The transcriptional and translational regulatory nucleic acid are generally appropriate for the host cell used for expression. Numerous types of appropriate expression vectors and suitable regulatory sequences are known in the art for a variety of host cells. Typically, the transcriptional and translational regulatory nucleic acid may include, but is not limited to, promoter sequences, leader or signal sequences, ribosomal binding sites, transcriptional start and stop sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter.

In a preferred embodiment, the expression vector contains a selectable marker gene to allow the selection of transformed host cells. Selection genes are well known in the art and are chosen according to the choice of the particular host cell used.

The expression vector may also include a fusion partner (typically provided by the expression vector) so that the recombinant polypeptide of the invention is expressed as a fusion polypeptide with said fusion partner. The main advantage of fusion partners is that they assist identification and/or purification of said fusion polypeptide. In order to express said fusion polypeptide, it is necessary to ligate a polynucleotide according to the invention into the expression vector so that the translational reading frames of the fusion partner and the polynucleotide coincide. Well known examples of fusion partners include, but are not limited to, glutathione-S-transferase (GST), Fc potion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. In a preferred embodiment, the recombinant polynucleotide is expressed in the commercial vector pFLAG as described more fully hereinafter.

Another fusion partner well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localisation of the fusion polypeptide of the invention, or for isolating cells which express the fusion polypeptide of the invention. Flow cytometric methods such as fluorescence activated cell sorting (FACS) are particularly useful in this latter application.

Preferably, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation.

Fusion partners according to the invention also include within their scope “epitope tags”, which are usually short peptide sequences for which a specific antibody is available. Well known examples of epitope tags for which specific monoclonal antibodies are readily available include c-Myc, influenza virus, haemagglutinin and FLAG tags.

The step of introducing into the host cell the recombinant polynucleotide may be effected by any suitable method including transfection, and transformation, the choice of which is dependent on the host cell employed. Such methods are well known to those of skill in the art.

Recombinant polypeptides of the invention may be produced by culturing a host cell transformed with an expression vector containing nucleic acid encoding a polypeptide, biologically active fragment, variant or derivative according to the invention. The conditions appropriate for protein expression will vary with the choice of expression vector and the host cell. This is easily ascertained by one skilled in the art through routine experimentation.

Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

The recombinant protein may be conveniently prepared by a person skilled in the art using standard protocols as for example described in Sambrook, et al., 1989, in particular Sections 16 and 17; Ausubel et al., (1994-1998), in particular Chapters 10 and 16; and Coligan et al., (1995-1997), in particular Chapters 1, 5 and 6.

Alternatively, the polypeptide, fragments, variants or derivatives of the invention may be synthesised using solution synthesis or solid phase synthesis as described, for example, in Chapter 9 of Atherton and Shephard (supra) and in Roberge et al (1995).

3. Polynucleotides of the Invention

3.1 Polynucleotides Encoding Polypeptides of the Invention

The invention further provides a polynucleotide that encodes a polypeptide, fragment, variant or derivative as defined above. In one embodiment, the polynucleotide comprises the entire sequence of nucleotides set forth in SEQ ID NO: 1. SEQ ID NO: 1 corresponds to the full-length murine 3472 bp Sox18 genomic sequence. This sequence defines: (1) a first exon from nucleotide 1 through nucleotide 2130; (2) an intron from nucleotide 2131 through nucleotide 2314; (3) a second exon from nucleotide 2315 through nucleotide 3472; (4) a 5′ untranslated region from nucleotide 1 through nucleotide 1516; (5) an open reading frame from nucleotide 1517 through nucleotide 2128; (6) another open reading frame from nucleotide 2315 through nucleotide 3109; and (7) a 3′ untranslated region from nucleotide 3110 through nucleotide 3472. The aforementioned open reading frames, when joined together, encode a polypeptide comprising 468 residues set forth in SEQ ID NO: 2.

The mouse Sox18 gene and the human SOX18 gene discussed more fully hereinafter, as well as portions thereof and flanking polynucleotide sequences have utility for isolating or otherwise produce polynucleotide sequences, including genomic and cDNA sequences of other animals, which could be taken advantage to produce non-human transgenic animals. Useful sequences for producing transgenic animals include, but are not restricted to, open reading frames encoding specific polypeptides or domains, introns, and adjacent 5′ and 3′ non-coding nucleotide sequences involved in the regulation of expression, up to about 1 kb beyond the coding region, but possibly further in either direction.

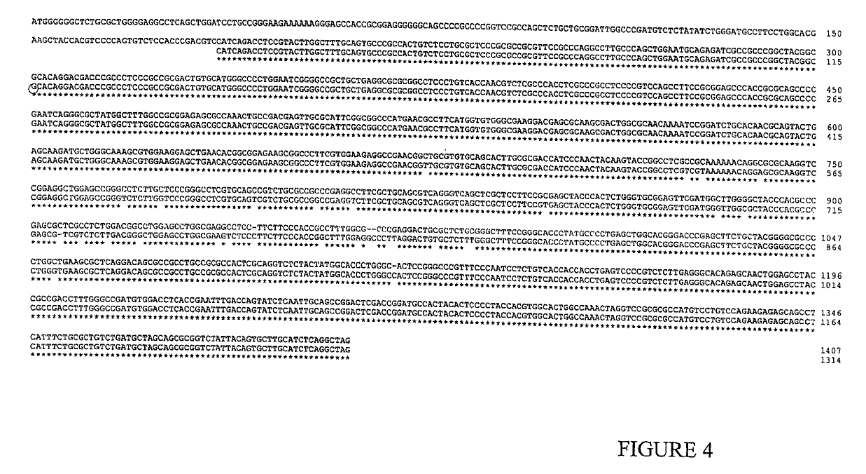

Preferably, the polynucleotide comprises the sequence set forth in SEQ ID NO: 3, which defines the Sox18 exonic sequence, wherein the intron has been removed. Alternatively, the polynucleotide comprises the sequence set forth in SEQ ID NO: 5, which defines the entire coding sequence of Sox18. An alignment of the previously published mouse Sox18 coding sequence with the novel mouse Sox18 coding sequence disclosed herein is presented in FIG. 4. The coding sequence of the present invention comprises an additional 185 bp of sequence at the 5′ end, 19 nucleotide substitutions, one insertion and four deletions relative to the known mouse Sox18 sequence.

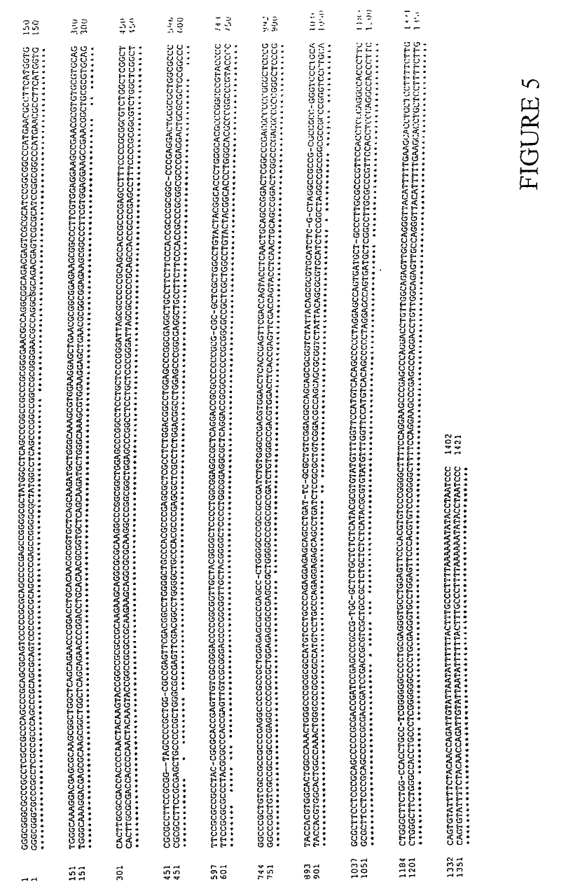

In another embodiment, the polynucleotide comprises the entire sequence of nucleotides set forth in SEQ ID NO: 14, which corresponds to a partial human Sox18 cDNA sequence. As mentioned in Section 2.1 supra, this partial human Sox18 cDNA sequence was surprisingly discovered as a result of resequencing a cDNA molecule, which was previously published by Stevens et al. (1996, supra) as encoding HAF-2, a transcription factor that is putatively involved in establishing the B cell activity of the human immunoglobulin heavy chain enhancer. The present inventors found that upon resequencing, the HAF-2 nucleotide sequence contained 33 sequencing errors made of 14 substitutions and 19 insertions. SEQ ID NO: 14 defines: (1) a partial coding sequence from nucleotide 1 through nucleotide 1023, which encodes the amino acid sequence set forth in SEQ ID NO: 15; and (2) a 3′ untranslated region from nucleotide 1024 through nucleotide 1420. An alignment of the known HAF-2 nucleotide sequence with the novel human Sox18 nucleotide sequence is shown in FIG. 5. In an alternate embodiment, the polynucleotide comprises the sequence set forth in SEQ ID NO: 16, which defines the coding sequence of SEQ ID NO: 14.

The invention also provides an isolated polynucleotide comprising a human Sox18 gene. Preferably, the polynucleotide comprises the entire sequence of nucleotides set forth in SEQ ID NO: 17, which corresponds to a 1919 bp human genomic sequence for Sox18. This sequence defines: (1) a first exon from nucleotide 1 through nucleotide 482, comprising a 5′ untranslated region from nucleotide 1 through nucleotide 125 and a 5′ portion of the Sox18 ORF from nucleotide 126 through nucleotide 482; (2) an intron from nucleotide 483 through nucleotide 678; (3) a second exon from nucleotide 679 through nucleotide 1919, comprising the remaining portion of the Sox18 ORF, from nucleotide 679 through nucleotide 1473. The Sox18 gene and portions thereof, including exons and introns, have utility in a variety of applications, including its use in identifying aberrant SOX18 genes and transcripts that associate with enhancement or promotion of an activity mentioned above.

In another embodiment, the polynucleotide comprises the entire sequence of nucleotides set forth in SEQ ID NO: 19, which corresponds to the full-length human 1730 bp -human SOX18 cDNA sequence. This sequence defines: (1) a 5′ untranslated region from nucleotide 1 through nucleotide 122; (2) an open reading frame from nucleotide 123 through nucleotide 1277; and (3) a 3′ untranslated region from nucleotide 1278 through nucleotide 1730. Preferably, the polynucleotide comprises the sequence set forth in SEQ ID NO: 21, which defines the entire ORF of human SOX18.

3.2 Polynucleotides Variants

In general, polynucleotide variants according to the invention comprise regions that show at least 85%, more preferably at least 90%, preferably at least 95%, and more preferably at least 98% sequence identity over a reference polynucleotide sequence of identical size (“comparison window”) or when compared to an aligned sequence in which the alignment is performed by a computer homology program known in the art. What constitutes suitable variants may be determined by conventional techniques. For example, a polynucleotide according to any one of SEQ ID NO: 11, 3, 5, 14, 16, 17, 19 and 21 can be mutated using random mutagenesis (e.g., transposon mutagenesis), oligonucleotide-mediated (or site-directed) mutagenesis, PCR mutagenesis and cassette mutagenesis of an earlier prepared variant or non-variant version of an isolated natural promoter according to the invention.

Oligonucleotide-mediated mutagenesis is a preferred method for preparing nucleotide substitution variants of a polynucleotide of the invention. This technique is well known in the art as, for example, described by Adelman et al. (1983). Briefly, a polynucleotide according to any one of SEQ ID NO: 1, 3, 5, 14, 16, 17, 19 and 21 is altered by hybridising an oligonucleotide encoding the desired mutation to a template DNA, wherein the template is the single-stranded form of a plasmid or bacteriophage containing the unaltered or parent DNA sequence. After hybridisation, a DNA polymerase is used to synthesise an entire second complementary strand of the template that will thus incorporate the oligonucleotide primer, and will code for the selected alteration in said parent DNA sequence.

Generally, oligonucleotides of at least 25 nucleotides in length are used. An optimal oligonucleotide has 12 to 15 nucleotides that are completely complementary to the template on either side of the nucleotide(s) coding for the mutation. This ensures that the oligonucleotide hybridises properly to the single-stranded DNA template molecule.

The DNA template can be generated by those vectors that are either derived from bacteriophage M13 vectors, or those vectors that contain a single-stranded phage origin of replication as described by Viera et al. (1987). Thus, the DNA that is to be mutated may be inserted into one of the vectors to generate single-stranded template. Production of single-stranded template is described, for example, in Sections 4.21-4.41 of Sambrook et al. (1989, supra).

Alternatively, the single-stranded template may be generated by denaturing double-stranded plasmid (or other DNA) using standard techniques.

For alteration of the native DNA sequence, the oligonucleotide is hybridised to the single-stranded template under suitable hybridisation conditions. A DNA polymerising enzyme, usually the Klenow fragment of DNA polymerase I, is then added to synthesise the complementary strand of the template using the oligonucleotide as a primer for synthesis. A heteroduplex molecule is thus formed such that one strand of DNA encodes the mutated form of the polypeptide or fragment under test, and the other strand (the original template) encodes the native unaltered sequence of the polypeptide or fragment under test. This heteroduplex molecule is then transformed into a suitable host cell, usually a prokaryote such as E. coli. After the cells are grown, they are plated onto agarose plates and screened using the oligonucleotide primer having a detectable label to identify the bacterial colonies having the mutated DNA. The resultant mutated DNA fragments are then cloned into suitable expression hosts such as E. coli using conventional technology and clones that retain the desired antigenic activity are detected. Where the clones have been derived using random mutagenesis techniques, positive clones would have to be sequenced in order to detect the mutation.

Alternatively, linker-scanning mutagenesis of DNA may be used to introduce clusters of point mutations throughout a sequence of interest that has been cloned into a plasmid vector. For example, reference may be made to Ausubel et al., supra, (in particular, Chapter 8.4) which describes a first protocol that uses complementary oligonucleotides and requires a unique restriction site adjacent to the region that is to be mutagenised. A nested series of deletion mutations is first generated in the region. A pair of complementary oligonucleotides is synthesised to fill in the gap in the sequence of interest between the linker at the deletion endpoint and the nearby restriction site. The linker sequence actually provides the desired clusters of point mutations as it is moved or “scanned” across the region by its position at the varied endpoints of the deletion mutation series. An alternate protocol is also described by Ausubel et al., supra, which makes use of site directed mutagenesis procedures to introduce small clusters of point mutations throughout the target region. Briefly, mutations are introduced into a sequence by annealing a synthetic oligonucleotide containing one or more mismatches to the sequence of interest cloned into a single-stranded M13 vector. This template is grown in an E. coli dut⁻ ung⁻ strain, which allows the incorporation of uracil into the template strand. The oligonucleotide is annealed to the template and extended with T4 DNA polymerase to create a double-stranded heteroduplex. Finally, the heteroduplex is introduced into a wild-type E. coli strain, which prevents replication of the template strand due to the presence of apurinic sites (generated where uracil is incorporated), thereby resulting in plaques containing only mutated DNA.

Region-specific mutagenesis and directed mutagenesis using PCR may also be employed to construct polynucleotide variants according to the invention. In this regard, reference may be made, for example, to Ausubel et al., supra, in particular Chapters 8.2A and 8.5.

Alternatively, suitable polynucleotide sequence variants of the invention may be prepared according to the following procedure:

creating primers which are optionally degenerate wherein each comprises a portion of a reference polynucleotide encoding a reference polypeptide or fragment of the invention, preferably encoding the sequence set forth in any one of SEQ ID NO: 2, 15 and 18;

obtaining a nucleic acid extract from an organism, which is preferably an animal, and more preferably a mammal; and

using said primers to amplify, via nucleic acid amplification techniques, at least one amplification product from said nucleic acid extract, wherein said amplification product corresponds to a polynucleotide variant.

Suitable nucleic acid amplification techniques are well known to the skilled addressee, and include polymerase chain reaction (PCR) as for example described in Ausubel et al. (supra); strand displacement amplification (SDA) as for example described in U.S. Pat. No 5,422,252; rolling circle replication (RCR) as for example described in Liu et al., (1996) and International application WO 92/01813) and Lizardi et al., (International Application WO 97/19193); nucleic acid sequence-based amplification (NASBA) as for example described by Sooknanan et al., (1994); and Q-β replicase amplification as for example described by Tyagi et al., (1996).

Typically, polynucleotide variants that are substantially complementary to a reference polynucleotide are identified by blotting techniques that include a step whereby nucleic acids are immobilised on a matrix (preferably a synthetic membrane such as nitrocellulose), followed by a hybridisation step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al. (1994-1998, supra) at pages 2.9.1 through 2.9.20.

According to such methods, Southern blotting involves separating DNA molecules according to size by gel electrophoresis, transferring the size-separated DNA to a synthetic membrane, and hybridising the membrane-bound DNA to a complementary nucleotide sequence labelled radioactively, enzymatically or fluorochromatically. In dot blotting and slot blotting, DNA samples are directly applied to a synthetic membrane prior to hybridisation as above.

An alternative blotting step is used when identifying complementary polynucleotides in a cDNA or genomic DNA library, such as through the process of plaque or colony hybridisation. A typical example of this procedure is described in Sambrook et al (1989) Chapters 8-12.

Typically, the following general procedure can be used to determine hybridisation conditions. Polynucleotides are blotted/transferred to a synthetic membrane, as described above. A reference polynucleotide such as a polynucleotide of the invention is labelled as described above, and the ability of this labelled polynucleotide to hybridise with an immobilised polynucleotide is analysed.

A skilled artisan recognises that a number of factors influence hybridisation. The specific activity of radioactively labelled polynucleotide sequence should typically be greater than or equal to about 10⁸ dpm/mg to provide a detectable signal. A radiolabeled nucleotide sequence of specific activity 10⁸ to 10⁹ dpm/mg can detect approximately 0.5 pg of DNA. It is well known in the art that sufficient DNA must be immobilised on the membrane to permit detection. It is desirable to have excess immobilised DNA, usually 10 μg. Adding an inert polymer such as 10% (w/v) dextran sulfate (MW 500,000) or polyethylene glycol 6000 during hybridisation can also increase the sensitivity of hybridisation (see Ausubel supra at 2.10.10).

To achieve meaningful results from hybridisation between a polynucleotide immobilised on a membrane and a labelled polynucleotide, a sufficient amount of the labelled polynucleotide must be hybridised to the immobilised polynucleotide following washing. Washing ensures that the labelled polynucleotide is hybridised only to the immobilised polynucleotide with a desired degree of complementarity to the labelled polynucleotide.

It will be understood that polynucleotide variants according to the invention hybridise to a reference polynucleotide under at least low stringency conditions. Reference herein to low stringency conditions include and encompass from at least about 1% v/v to at least about 15% v/v formamide and from at least about 1 M to at least about 2 M salt for hybridisation at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridisation at 65° C., and (i) 2× SSC, 0.1% SDS; or (ii) 0.5% BSA, 1 mM EDTA, 40 mM NaHPO₄ (pH 7.2), 5% SDS for washing at room temperature.

Preferably, the polynucleotide variants hybridise to a reference polynucleotide under at least medium stringency conditions. Medium stringency conditions include and encompass from at least about 16% v/v to at least about 30% v/v formamide and from at least about 0.5 M to at least about 0.9 M salt for hybridisation at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridisation at 65° C., and (i) 2× SSC, 0.1% SDS; or (ii) 0.5% BSA, 1 mM EDTA, 40 mM NaHPO₄ (pH 7.2), 5% SDS for washing at 60-65° C.

Preferably, the polynucleotide variants hybridise to a reference polynucleotide under high stringency conditions. High stringency conditions include and encompass from at least about 31% v/v to at least about 50% v/v formamide and from about 0.01 M to about 0.15 M salt for hybridisation at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridisation at 65° C., and (i) 0.2× SSC, 0.1% SDS; or (ii) 0.5% BSA, 1 mM EDTA, 40 mM NaHPO₄ (pH 7.2), 1% SDS for washing at a temperature in excess of 65° C.

Other stringent conditions are well known in the art. A skilled artisan recognises that various factors can be manipulated to optimise the specificity of the hybridisation. Optimisation of the stringency of the final washes can serve to ensure a high degree of hybridisation. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridisation rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula:

Tₘ=81.5+16.6(log₁₀M)+0.41(%G+C)−0.63(%formamide)−(600/length)

wherein: M is the concentration of Na⁺, preferably in the range of 0.01 molar to 0.4 molar; %G+C is the sum of guanosine and cytosine bases as a percentage of the total number of bases, within the range between 30% and 75% G+C; % formamide is the percent formamide concentration by volume; length is the number of base pairs in the DNA duplex.

The Tₘ of a duplex DNA decreases by approximately 1° C. with every increase of 1% in the number of randomly mismatched base pairs. Washing is generally carried out at Tₘ −15° C. for high stringency, or Tₘ−30° C. for moderate stringency.

In a preferred hybridisation procedure, a membrane (e.g., a nitrocellulose membrane or a nylon membrane) containing immobilised DNA is hybridised overnight at 42° C. in a hybridisation buffer (50% deionised formamide, 5× SSC, 5× Denhardt's solution (0.1% ficoll, 0.1% polyvinylpyrollidone and 0.1% bovine serum albumin), 0.1% SDS and 200 mg/mL denatured salmon sperm DNA) containing labelled probe. The membrane is then subjected to two sequential medium stringency washes (i.e., 2× SSC, 0.1% SDS for 15 min at 45° C., followed by 2× SSC, 0.1% SDS for 15 min at 50° C.), followed by two sequential higher stringency washes (i.e., 0.2× SSC, 0.1% SDS for 12 min at 55° C. followed by 0.2× SSC and 0.1% SDS solution for 12 min at 65-68° C.

Methods for detecting a labelled polynucleotide hybridised to an immobilised polynucleotide are well known to practitioners in the art. Such methods include autoradiography, phosphorimaging, and chemiluminescent, fluorescent and colorimetric detection.

4. Antigen-binding Molecules

The invention also contemplates antigen-binding molecules that bind specifically to the aforementioned polypeptides, fragments, variants and derivatives. For example, the antigen-binding molecules may comprise whole polyclonal antibodies. Such antibodies may be prepared, for example, by injecting a polypeptide, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., (1991), and Ausubel et al., (1994-1998, supra), in particular Section III of Chapter 11.

In lieu of the polyclonal antisera obtained in the production species, monoclonal antibodies may be produced using the standard method as described, for example, by Köhler and Milstein (1975), or by more recent modifications thereof as described, for example, in Coligan et al., (1991, supra) by immortalising spleen or other antibody producing cells derived from a production species which has been inoculated with one or more of the polypeptides, fragments, variants or derivatives of the invention.

The invention also contemplates as antigen-binding molecules Fv, Fab, Fab′ and F(ab′)₂ immunoglobulin fragments.

Alternatively, the antigen-binding molecule may comprise a synthetic stabilised Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Kreber et al (1997). Alternatively, they may be prepared by methods described in U.S. Pat. No. 5,091,513, European Patent No 239,400 or the articles by Winter and Milstein (1991) and Plückthun et al (1996).

Alternatively, the synthetic stabilised Fv fragment comprises a disulphide stabilised Fv (dsFv) in which cysteine residues are introduced into the V_{H} and V_{L} domains such that in the fully folded Fv molecule the two residues form a disulphide bond therebetween. Suitable methods of producing dsFv are described for example in (Glockscuther et al.; Reiter et al. 1994¹; Reiter et al. 1994²; Reiter et al. 1994³; Webber et al. 1995).

Also contemplated as antigen-binding molecules are single variable region domains (termed dAbs) as for example disclosed in (Ward et al. 1989; Hamers-Casterman et al. 1993; Davies & Riechmann, 1994).

Alternatively, the antigen-binding molecule may comprise a “minibody”. In this regard, minibodies are small versions of whole antibodies, which encode in a single chain the essential elements of a whole antibody. Preferably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the antigen binding molecule may comprise non-immunoglobulin derived, protein frameworks. For example, reference may be made to (Ku & Schultz, 1995) which discloses a four-helix bundle protein cytochrome b562 having two loops randomised to create complementarity determining regions (CDRs), which have been selected for antigen binding.

The antigen-binding molecule may be multivalent (i.e., having more than one antigen binding site). Such multivalent molecules may be specific for one or more antigens. Multivalent molecules of this type may be prepared by dimerisation of two antibody fragments through a cysteinyl-containing peptide as, for example disclosed by (Adams et al., 1993; Cumber et al., 1992). Alternatively, dimerisation may be facilitated by fusion of the antibody fragments to amphiphilic helices that naturally dimerise (Pack P. Plünckthun, 1992), or by use of domains (such as the leucine zippers jun and fos) that preferentially heterodimerise (Kostelny et al., 1992). In an alternate embodiment, the multivalent molecule may comprise a multivalent single chain antibody (multi-scFv) comprising at least two scFvs linked together by a peptide linker. In this regard, non-covalently or covalently linked scFv dimers termed “diabodies” may be used. Multi-scFvs may be bispecific or greater depending on the number of scFvs employed having different antigen binding specificities. Multi-scFvs may be prepared for example by methods disclosed in U.S. Pat. No. 5,892,020.

The antigen-binding molecules of the invention may be used for affinity chromatography in isolating a natural or recombinant polypeptide or biologically active fragment of the invention. For example reference may be made to immunoaffinity chromatographic procedures described in Chapter 9.5 of Coligan et al., (1995-1997, supra).

The antigen-binding molecules can be used to screen expression libraries for variant polypeptides of the invention as described herein. They can also be used to detect polypeptides, fragments, variants and derivatives of the invention as described hereinafter.

5. Methods of Detection

5.1 Detection of Polypeptides according to the Invention

The invention also extends to a method of detecting in a sample a polypeptide, fragment, variant or derivative as broadly described above, comprising contacting the sample with an antigen-binding molecule as described in Section 4 and detecting the presence of a complex comprising the said antigen-binding molecule and the said polypeptide, fragment, variant or derivative in said contacted sample.

Any suitable technique for determining formation of the complex may be used. For example, an antigen-binding molecule according to the invention, having a reporter molecule associated therewith may be utilised in immunoassays. Such immunoassays include, but are not limited to, radioimmunoassays (RIAs), enzyme-linked immunosorbent assays (ELISAs) and immunochromatographic techniques (ICTs), Western blotting which are well known those of skill in the art. For example, reference may be made to “CURRENT PROTOCOLS IN IMMUNOLOGY” (1994, supra) which discloses a variety of immunoassays that may be used in accordance with the present invention. Immunoassays may include competitive assays as understood in the art or as for example described infra. It will be understood that the present invention encompasses qualitative and quantitative immunoassays.

Suitable immunoassay techniques are described for example in U.S. Pat. Nos. 4,016,043, 4,424,279 and 4,018,653. These include both single-site and two-site assays of the non-competitive types, as well as the traditional competitive binding assays. These assays also include direct binding of a labelled antigen-binding molecule to a target antigen.

Two site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilised on a solid substrate and the sample to be tested brought into contact with the bound molecule. After a suitable period of incubation, for a period of time sufficient to allow formation of an antibody-antigen complex, another antigen-binding molecule, preferably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent. In accordance with the present invention, the sample is one that might contain an antigen including a tissue sample or biopsy, including, but not limited to, cardiovascular tissue, skin and lung tissue.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilised first antibody.

An alternative method involves immobilising the antigen in the biological sample and then exposing the immobilised antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:

(a) direct attachment of the reporter molecule to the antigen-binding molecule;

(b) indirect attachment of the reporter molecule to the antigen-binding molecule; i.e., attachment of the reporter molecule to another assay reagent which subsequently binds to the antigen-binding molecule; and

(c) attachment to a subsequent reaction product of the antigen-binding molecule.

The reporter molecule may be selected from a group including a chromogen, a catalyst, an enzyme, a fluorochrome, a chemiluminescent molecule, a lanthanide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in U.S. Pat. Nos. 4,366,241, 4,843,000, and 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. Nos. 5,573,909 (Singer et al), U.S. Pat. No. 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodate. As will be readily recognised, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex. It is then allowed to bind, and excess reagent is washed away. A solution containing the appropriate substrate is then added to the complex of antibody-antigen-antibody. The substrate reacts with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art. However, other reporter molecules, such as radioisotope, chemiluminescent or bioluminescent molecules may also be employed.

5.2 Detection of Polynucleotides according to the Invention

In another embodiment, the method for detection comprises detecting a polynucleotide encoding said polypeptide, fragment, variant or derivative of the invention. The polynucleotide may correspond to an expression product of the Sox18 gene. Expression of the polynucleotide may be determined using any suitable technique. For example, a labelled polynucleotide encoding a said member may be utilised as a probe in a Northern blot of a RNA extract obtained from the muscle cell. Preferably, a nucleic acid extract from the animal is utilised in concert with oligonucleotide primers corresponding to sense and antisense sequences of a polynucleotide encoding a said member, or flanking sequences thereof, in a nucleic acid amplification reaction such as RT PCR. A variety of automated solid-phase detection techniques are also appropriate. For example, very large scale immobilised primer arrays (VLSIPS™ ) are used for the detection of nucleic acids as for example described by Fodor et al., (1991) and Kazal et al., (1996). The above generic techniques are well known to persons skilled in the art.

6. Screening for Modulators of Sox18

The present invention is predicated in part on the discovery that SOX18 is necessary for angiogenesis and vasculogenesis. Vascular growth and remodelling is an important part of many physiological processes, such as embryogenesis, ovulation and the menstrual cycle, and a key component of many pathological conditions such as diabetic retinopathy, artherosclerosis, pulmonary disease, restenosis, wound healing and tumorigenesis. The control of vasculogenesis and angiogenesis has become one of the major issues in biomedical science, and is the focus of intense pharmaceutical interest. Accordingly, it is believed that modulation of the level and/or functional activity of SOX18, inclusive of fragments, variants and derivatives of SOX18, or modulation of expression of genes encoding these molecules could modulate, for example, one or more of the above physiological processes and/or which can modulate an activity selected from the group consisting of cell differentiation, vasculogenesis, angiogenesis, hair follicle development, cell proliferation and tumorigenesis.

In a preferred embodiment, the modulation of the aforementioned activities is effected by also modulating other subgroup F SOX proteins, including, but not restricted to, SOX7 and SOX17.

Any suitable assay for detecting, measuring or otherwise determining modulation of cell differentiation, vasculogenesis, angiogenesis, hair follicle development, cell proliferation, and tumorigenesis is contemplated by the present invention. Assays of a suitable nature are known to persons of skill in the art. It will be understood, in this regard, that the present invention is not limited to the use or practice of any one particular assay for determining an activity mentioned above.

Typically, for cell proliferation, cell number is determined, directly, by microscopic or electronic enumeration, or indirectly, by the use of chromogenic dyes, incorporation of radioactive precursors or measurement of metabolic activity of cellular enzymes. An exemplary cell proliferation assay comprises culturing cells in the presence or absence of a test compound, and detecting cell proliferation by, for example, measuring incorporation of tritiated thymidine or by colorimetric assay based on the metabolic breakdown of 3-(4,5-dimethylthiazol-2-yl)-2,5-diphenyl tetrazolium bromide (MTT) (Mosman, 1983, J. Immunol. Meth. 65: 55-63).

Cell differentiation may be assessed in several different ways. One such method is by measuring cell phenotypes. The phenotypes of cells and any phenotypic changes can be evaluated by flow cytometry after immunofluorescent staining using monoclonal antibodies that bind membrane proteins characteristic of various cell types. A second means of assessing cell differentiation is by measuring cell function. This may be done biochemically, by measuring the expression of enzymes, mRNAs, genes, proteins, or other metabolites within the cell, or secreted from the cell. Bioassays may also be used to measure functional cell differentiation.

Compounds of interest may be tested for suitability as inhibitors of cell proliferation and enhancers of differentiation using cultured human keratinocytes, as described, for example, in U.S. Pat. No. 5,037,816. Those compounds which inhibit proliferation and induce differentiation in cultured keratinocytes are those potentially useful as therapeutic agents in treating disorders, e.g., precancer, such as actinic keratoses, and cancer, where suppression of cell proliferation is desired.

Cells including, but not restricted to, cardiovascular cells, lung cells, and skin cells, express a variety of cell surface molecules which can be detected with monoclonal antibodies or polyclonal antisera or other antigen-binding molecules. Cells that have undergone differentiation can also be enumerated by staining for the presence of characteristic cell surface proteins by direct immunofluorescence in fixed smears of cultured cells.

Angiogenesis may be assessed, for example, using the method of Parish et al (U.S. Pat. No. 5,976,782). In this assay a compound of interest is cultured in the presence of a blood vessel fragment derived from either venular or arterial origin and a physiological gel (e.g., fibrin, collagen or Matrigel™) suitable nutrients for a time and under conditions sufficient to allow growth of new vascular tissue. The blood vessel fragment is then examined for new vascular tissue growth, and this growth is compared to a control blood vessel fragment, which is cultured in the absence of the compound of interest. Pro-angiogenesis compounds test positive if there is enhanced growth of vascular tissue relative to the control. Conversely, anti-angiogenesis compounds test positive if there is reduced growth of vascular tissue relative to the control.

Alternatively, angiogenesis may be assessed by use of the corneal micropocket angiogenesis assay (CMA). The rat corneal micropocket assay can be used to assess the ability of the compounds to inhibit corneal angiogenesis (see, “Quantitative Angiogenesis Assays: Progress and Problems,” Nat. Med., 3: 1203-1208 (1997) and “Inhibition of Tumor Angiogenesis Using a Soluble Receptor Establishes a Role for Tie2 in Pathologic Vascular Growth.” J. Clin. Invest., 100: 2072-2078. (1997)). In this assay, a compound of interest is mixed with a polymer (e.g., Hydron solution; Interferon Sciences, New Brunswick, N.J.) and implanted in a small pocket surgically created in the superficial layers of the cornea of a rat. Under normal circumstances, this wound stimulates an angiogenic response, which is readily visible as the appearance of neovessels on the normally avascular cornea. If the compound is effective, specifically as an anti-angiogenic agent, it inhibits or blocks this response. In one experimental design, a group of five animals (including a control group with only polymer implants) is tested over a range of drug doses which can induce tumor growth delay. Three doses are tested in the assay. Assessment of an anti-angiogenic response by this method is categorical. In other words, a treated eye is either positive or negative for corneal angiogenesis. This assay determines whether a compound of interest is directly anti-angiogenic in an in vivo mammalian model of angiogenesis.

Candidate compounds may be tested for their ability to enhance wound healing by carrying out a skin punch biopsy test as described, for example, in International Publication No. W092/04039.

Vasculogenesis may be tested by histological study of embryos as for example described in the Experimental section, infra.

Compounds of interest may be tested for their suitability as modulators of hair follicle development or hair growth using an in vitro hair growth assay as, for example, described in International Publication No. WO092/04039. Alternatively, such compounds may be tested in a human hair follicle growth assay as, for example, described in U.S. Pat. No. 6,121,269. Briefly, hair follicles in growth phase (anagen) are cultured in the presence of a compound of interest for a time and under conditions sufficient to allow hair follicle growth. The hair follicles are then examined for growth (e.g., length of hair follicle), and this growth is compared to control hair follicles, which are cultured in the absence of the compound of interest. Promoters of hair follicle growth test positive if there is enhanced growth of hair relative to the control. Conversely, inhibitors of hair growth test positive if there is reduced growth of hair relative to the control.

Cancer or tumor markers are known for a variety of cell or tissue types. Cells or tissues expressing cancer or tumor markers may be detected using monoclonal antibodies, polyclonal antisera or other antigen-binding molecules that are immuno-interactive with these markers or by using nucleic acid analysis techniques, including, for example, detecting the level or presence of marker-encoding polynucleotides.

Modulators contemplated by the present invention includes agonists and antagonists of any one or more of Sox7, Sox17 and Sox18 gene expression. Antagonists of Sox7, Sox17 or Sox18 gene expression include antisense molecules, ribozymes and co-suppression molecules. Agonists include molecules which increase promoter activity or interfere with negative mechanisms. Agonists of Sox7, Sox17 or Sox18 include molecules which overcome any negative regulatory mechanism. Antagonists of Sox7, Sox17 or Sox18 polypeptides include antibodies and inhibitor peptide fragments. Another class of modulators may be designed to mimic or block trans-activation by SOX7, SOX17 or Sox18 polypeptides.

The invention therefore provides a method for screening for an agent which modulates one or more of the conditions or activities mentioned above, comprising contacting a preparation comprising a SOX7, SOX17 or SOX18 polypeptide as broadly described above or a genetic sequence encoding said polypeptide with a test agent; and detecting a change in the level and/or functional activity of said polypeptide or an expression product of said genetic sequence.

Candidate agents encompass numerous chemical classes, though typically they are organic molecules, preferably small organic compounds having a molecular weight of more than 50 and less than about 2,500 Dalton. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogues or combinations thereof.

Small (non-peptide) molecule modulators of SOX7, SOX17 and/or SOX18 are particularly preferred. In this regard, small molecules are particularly preferred because such molecules are more readily absorbed after oral administration, have fewer potential antigenic determinants, and/or are more likely to cross the cell membrane than larger, protein-based pharmaceuticals. Small organic molecules may also have the ability to gain entry into an appropriate cell and affect the expression of a gene (e.g., by interacting with the regulatory region or transcription factors involved in gene expression); or affect the activity of a gene by inhibiting or enhancing the binding of accessory molecules.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogues.

Screening may also be directed to known pharmacologically active compounds and chemical analogues thereof.

Screening for modulatory agents according to the invention can be achieved by any suitable method. For example, the method may include contacting a cell, preferably an endothelial cell, comprising a genetic sequence from which a target protein such as SOX7, SOX17 or SOX18 can be translated, with an agent suspected of having said modulatory activity and screening for the modulation of that protein, or the modulation of expression of the genetic sequence encoding that protein, or the modulation of the activity or expression of a downstream cellular target of said protein. Detecting such modulation can be achieved utilising techniques including, but not restricted to, Western blotting, ELISA, and RT-PCR.

It will be understood that a genetic sequence from which the target protein of interest is regulated or expressed may be naturally occurring in the cell which is the subject of testing or it may have been introduced into the host cell for the purpose of testing. Further, the naturally-occurring or introduced sequence may be constitutively expressed—thereby providing a model useful in screening for agents which down-regulate expression of an encoded product of the sequence wherein said down regulation can be at the nucleic acid or expression product level—or may require activation—thereby providing a model useful in screening for agents that up-regulate expression of an encoded product of the sequence. Further, to the extent that a polynucleotide is introduced into a cell, that polynucleotide may comprise the entire coding sequence which codes for the target protein or it may comprise a portion of that coding sequence (e.g. a domain such as a protein or DNA binding domain, e.g., HMG box domain, trans-activation domain and CCT domain) or a portion that regulates expression of a product encoded by the polynucleotide (e.g., a promoter). For example, the promoter that is naturally associated with the genetic sequence may be introduced into the cell, which is the subject of testing. In this regard, where only the promoter is utilised, detecting modulation of the promoter activity can be achieved, for example, by operably linking the promoter to a suitable reporter polynucleotide including, but not restricted to, luciferase, β-galactosidase and CAT. Modulation of expression may be determined by measuring the activity associated with the reporter polynucleotide.

In another example, the subject of detection could be a downstream regulatory target of the target protein, rather than target protein itself or the reporter molecule operably linked to a promoter of a gene encoding a protein the expression of which is regulated by the target protein.

These methods provide a mechanism for performing high throughput screening of putative modulatory agents such as proteinaceous or non-proteinaceous agents comprising synthetic, combinatorial, chemical and natural libraries. These methods also facilitate the detection of agents which bind either the genetic sequence encoding the target protein or expression product itself or which modulate the expression of an upstream molecule, which subsequently modulates the expression of the genetic sequence encoding the target protein or expression product activity. Accordingly, these methods provide a mechanism of detecting agents, which either directly or indirectly modulate the expression and/or activity of a target protein according to the invention.

In a series of preferred embodiments, the present invention provides assays for identifying small molecules or other compounds (i.e., modulatory agents) which are capable of inducing or inhibiting the expression of Sox7, Sox17 or Sox18 or Sox7-, Sox17- or Sox18-related genes and proteins. The assays may be performed in vitro using non-transformed cells, immortalised cell lines, or recombinant cell lines. In addition, the assays may detect the presence of increased or decreased expression of Sox7, Sox17 or Sox18, or other Sox7-, Sox17- or Sox18-related genes or proteins on the basis of increased or decreased mRNA expression (using, for example, the nucleic acid probes disclosed herein), increased or decreased levels of SOX7, SOX17 or SOX18 or other SOX7-, SOX17- or SOX18-related protein products (using, for example, the anti-SOX7, -SOX17 SOX18 antigen binding molecules), or increased or decreased levels of expression of a reporter gene (e.g., β-galactosidase or luciferase) operably linked to a Sox7, Sox17 or Sox18 5′ regulatory region in a recombinant construct.

Thus, for example, one may culture cells known to express a particular Sox7, Sox17 or Sox18 and add to the culture medium one or more test compounds. After allowing a sufficient period of time (e.g., 6-72 hours) for the compound to induce or inhibit the expression of Sox7, Sox17 and/or Sox18, any change in levels of expression from an established baseline may be detected using any of the techniques described above and well known in the art. In particularly preferred embodiments, the cells are endothelial. Using the nucleic acid probes and/or antigen-binding molecules disclosed herein, detection of changes in the expression of Sox7, Sox17 or Sox18, and thus identification of the compound as an inducer or repressor of Sox7, Sox17 or Sox18 expression, requires only routine experimentation.

In particularly preferred embodiments, a recombinant assay is employed in which a reporter gene such a β-galactosidase or luciferase is operably linked to the 5′ regulatory regions of a Sox gene mentioned above. Such regulatory regions may be easily isolated and cloned by one of ordinary skill in the art in light of the present disclosure of the coding regions of these genes. The reporter gene and regulatory regions are joined in-frame (or in each of the three possible reading frames) so that transcription and translation of the reporter gene may proceed under the control of the Sox gene regulatory elements. The recombinant construct may then be introduced into any appropriate cell type although mammalian cells are preferred, and human cells are most preferred. The transformed cells may be grown in culture and, after establishing the baseline level of expression of the reporter gene, test compounds may be added to the medium. The ease of detection of the expression of the reporter gene provides for a rapid, high throughput assay for the identification of inducers and repressors of the Sox gene.

Compounds identified by this method have potential utility in modifying the expression of Sox7, Sox17 or Sox18, or other Sox7-, Sox17- or Sox18-related genes in vivo. These compounds may be further tested in the animal models to identify those compounds having the most potent in vivo effects. In addition, as described above with respect to small molecules having SOX7-, SOX17- or SOX18-binding activity, these molecules may serve as “lead compounds” for the further development of pharmaceuticals by, for example, subjecting the compounds to sequential modifications, molecular modelling, and other routine procedures employed in rational drug design.

In another embodiment, a method of identifying agents that inhibit SOX7, SOX17 or SOX18 activity is provided in which a purified preparation of a said SOX protein is incubated in the presence and absence of a candidate agent and the level and/or functional activity of the SOX protein is measured by a suitable assay.

For example, an inhibitor of a SOX protein of the subject invention can be identified by measuring the ability of a candidate agent to decrease SOX activity in a cell (e.g., an endothelial cell). In this method, a cell that is capable of expressing Sox7, Sox17 or Sox18 is exposed to, or cultured in the presence and absence of, the candidate agent under conditions, and an activity selected from the group consisting of cell differentiation, vasculogenesis, angiogenesis and hair follicle development is detected. An agent tests positive if the it inhibits any of these activities.

For SOX18, SOX18 -binding partner (e.g., MEF2C) interactions and screens for inhibitors can be carried out using the yeast Two-Hybrid system, which takes advantage of transcriptional factors that are composed of two physically separable, functional domains (Phizicky and Fields, 1994). The most commonly used is the yeast GAL4 transcriptional activator consisting of a DNA binding domain and a transcriptional activation domain. Two different cloning vectors are used to generate separate fusions of the GAL4 domains to genes encoding potential binding proteins. The fusion proteins are co-expressed, targeted to the nucleus and, if interactions occur, activation of a reporter gene (e.g., lacZ) produces a detectable phenotype. In the present case, for example, S. cerevisiae is co-transformed with a vector expressing a MEF2C (or other SOX18-binding partner)-GAL4 activation domain fusion and a vector expressing a SOX18-GAL4 binding domain fusion. If lacZ is used as the reporter gene, co-expression of the fusion proteins produces a blue colour. Small molecules or other candidate compounds which interfere with interaction of SOX18 and the MEF2C will result in loss of colour of the cells. This system could be used to screen for small molecules that inhibit the SOX18-MEF2C interaction. For example, reference may be made to the yeast Two-Hybrid systems as, for example, disclosed by Munder et al. (1999) and by Young et al. (1998), which are especially preferred to screen for small molecules (e.g., non-peptide drugs) that inhibit the SOX18-MEF2C interaction. Molecules thus identified by this system could then be re-tested in mammalian cells.

In yet another embodiment, random peptide libraries consisting of all possible combinations of amino acids attached to a solid phase support may be used to identify peptides that are able to bind to a SOX protein of the invention or to a functional domain thereof (e.g., an HMG box domain, trans-activation domain and/or a CCT domain). Identification of molecules that are able to bind to the SOX protein may be accomplished by screening a peptide library with a recombinant soluble SOX protein. The SOX protein may be purified, recombinantly expressed or synthesised by any suitable technique.

To identify and isolate the peptide/solid phase support that interacts and forms a complex with a SOX protein described herein, it is necessary to label or “tag” the SOX protein. For example, the SOX protein may be conjugated to any suitable reporter molecule, including enzymes such as alkaline phosphatase and horseradish peroxidase and fluorescent reporter molecules such as fluorescein isothyiocynate (FITC), phycoerythrin (PE) and rhodamine. Conjugation of any given reporter molecule, with the SOX protein, may be performed using techniques that are routine in the art. Alternatively, SOX expression vectors may be engineered to express a chimeric SOX protein containing an epitope for which an antigen-binding molecule exists. The epitope specific antigen-binding molecule may be tagged using methods well known in the art including labelling with enzymes, fluorescent dyes or coloured or magnetic beads as for example described in Section 5.

The “tagged” SOX conjugate is incubated with the random peptide library for 30 minutes to one hour at 22° C. to allow complex formation between the SOX protein and peptide species within the library. The library is then washed to remove any unbound SOX protein. If the SOX protein has been conjugated to alkaline phosphatase or horseradish peroxidase the whole library is poured into a petri dish containing a substrate for either alkaline phosphatase or peroxidase, for example, 5-bromo-4-chloro-3-indoyl phosphate (BCIP) or 3,3′,4,4″-diaminobenzidine (DAB), respectively. After incubating for several minutes, the peptide/solid phase-SOX complex changes colour, and can be easily identified and isolated physically under a dissecting microscope with a micromanipulator. If a fluorescent-tagged SOX molecule has been used, complexes may be isolated by fluorescent activated sorting. If a chimeric SOX protein expressing a heterologous epitope has been used, detection of the peptide/SOX complex may be accomplished by using a labelled epitope specific antigen-binding molecule. Once isolated, the identity of the peptide attached to the solid phase support may be determined by peptide sequencing.

7. Method of Modulating a SOX18-related Activity

The invention therefore provides a method for modulating at least one activity selected from the group consisting of cell differentiation, vasculogenesis, angiogenesis, hair follicle development, cell proliferation and tumorigenesis, comprising contacting said cell with an agent for a time and under conditions sufficient to modulate the level and/or functional activity of a polypeptide as broadly described above.

In one embodiment, the agent increases the level and/or functional activity of SOX18. In another embodiment, the agent increases the level and/or functional activity of SOX7 and/or SOX17. In this instance, the agent is preferably used to promote, augment or otherwise enhance an activity selected from the group consisting of cell differentiation, vasculogenesis, angiogenesis and hair follicle development. Any suitable Sox7, Sox17 and/or Sox18 inducers or stabilising/activating agents may be used in this regard and these can be identified or produced by methods for example disclosed in Section 6.

In an alternate embodiment, the agent decreases the level and/or functional activity of at least one SOX protein selected from the group consisting of SOX7, SOX17 and SOX18. In such a case, the agent is preferably used to reduce, repress or otherwise inhibit cell proliferation or tumorigenesis. Suitable SOX7, SOX17 and SOX18 inhibitors may be identified or produced by methods for example disclosed in Section 6.

For example, a suitable SOX18 inhibitor includes a defective SOX18 polypeptide. In this regard, the inventors have discovered that mutant SOX18 proteins produced by Ra, RaJ, RaOp, and Ragl mice (i.e., having defective trans-activation domains) as hereinafter described do not interact with MEF2C in an in vitro GST-pull down assay. This finding underscores the biological significance of the interaction between SOX18 and MEF2C, and suggests that Sox18 mutations in Ra mice act in a dominant-negative fashion. Accordingly, the present invention contemplates use of such defective or mutant SOX18 polypeptides or polynucleotides from which such polypeptides can be translated as inhibitors of SOX18 function.

Alternatively, the SOX protein inhibitor may comprise oligoribonucleotide sequences, that include anti-sense RNA and DNA molecules and ribozymes that function to inhibit the translation of SOX protein-encoding mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of a gene encoding a polypeptide according to the invention, are preferred.

Ribozymes are enzymatic RNA molecules capable of catalysing the specific cleavage of RNA. The mechanism of ribozyme action involves sequence specific hybridisation of the ribozyme molecule to complementary target RNA, followed by a endonucleolytic cleavage. Within the scope of the invention are engineered hammerhead motif ribozyme molecules that specifically and efficiently catalyse endonucleolytic cleavage of Sox RNA sequences.

Specific ribozyme cleavage sites within any potential RNA target are initially identified by scanning the target molecule for ribozyme cleavage sites, which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridisation with complementary oligonucleotides, using ribonuclease protection assays.

Both anti-sense RNA and DNA molecules and ribozymes may be prepared by any method known in the art for the synthesis of RNA molecules. These include techniques for chemically synthesising oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesise antisense RNA constitutively or inducibly, depending on the promoter used, can be introduced stably into cell lines.

Various modifications to the DNA molecules may be introduced as a means of increasing intracellular stability and half-life. Possible modifications include but are not limited to the addition of flanking sequences of ribo- or deoxy- nucleotides to the 5′ and/or 3′ ends of the molecule or the use of phosphorothioate or 2′ O-methyl rather than phosphodiesterase linkages within the oligodeoxyribonucleotide backbone.

8. Compositions

The polypeptides, fragments, variants and derivatives described in Section 2, the polynucleotides and polynucleotide variants described in Section 3, and the modulatory agents described in Section 6 and 7 (therapeutic agents) can be used as actives for the treatment or prophylaxis of various conditions as, for example, described below. These therapeutic agents can be administered to a patient either by themselves, or in pharmaceutical compositions where they are mixed with a suitable pharmaceutically acceptable carrier.

Accordingly, the invention also provides a composition for treatment and/or prophylaxis of at least one condition selected from the group consisting of artherosclerosis, restenosis, pulmonary disease, and tissue injury, comprising an agent selected from the group consisting of a polypeptide, fragment, variant or derivative as broadly described above, a polynucleotide from which said polypeptide, fragment, variant or derivative can be translated, and a modulatory agent that enhances the level and/or functional activity of at least one SOX protein selected from the group consisting of SOX7, SOX17 and SOX18, together with a pharmaceutically acceptable carrier.

The invention also provides a composition for treatment and/or prophylaxis of tumorigenesis, comprising a modulatory agent that reduces the level and/or functional activity of at least one SOX protein selected from the group consisting of SOX7, SOX17 and SOX18, together with a pharmaceutically acceptable carrier.

Depending on the specific conditions being treated, therapeutic agents may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the therapeutic agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunogenic compositions, vaccines and DNA vaccines.

The agents can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of agent administered to a patient should be sufficient to effect a beneficial response in the patient over time such as a reduction in the symptoms associated with the condition, which is preferably a condition selected from the group consisting of artherosclerosis, restenosis, pulmonary disease, tissue injury and tumorigenesis. The quantity of the agent(s) to be administered may depend on the subject to be treated inclusive of the age, sex, weight and general health condition thereof. In this regard, precise amounts of the agent(s) for administration depend on the judgement of the practitioner. In determining the effective amount of the agent to be administered in the treatment or prophylaxis of the condition, the physician may evaluate tissue levels of a polypeptide, fragment, variant or derivative of the invention, and progression of the disorder. In any event, those of skill in the art may readily determine suitable dosages of the therapeutic agents of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilisers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as., for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceutical which can be used orally include push-fit capsules made of gelatin, as well as soft, sealed capsules made of gelatin and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

Dosage forms of the therapeutic agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Therapeutic agents of the invention may be provided as salts with pharmaceutically compatible counterions. Pharmaceutically compatible salts may be formed with many acids, including but not limited to hydrochloric, sulphuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC50 as determined in cell culture (e.g., the concentration of a test agent, which achieves a half-maximal inhibition or enhancement of SOX7, SOX17 and/or SOX18 activity). Such information can be used to more accurately determine useful doses in humans.

Toxicity and therapeutic efficacy of such therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the patient's condition. (See for example Fingl et al., 1975, in “The Pharmacological Basis of Therapeutics”, Ch. 1 p1).

Dosage amount and interval may be adjusted individually to provide plasma levels of the active agent which are sufficient to maintain SOX18-inhibitory or enhancement effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m²/day, typically from 5-100 mg/m2 /day.

Alternately, one may administer the compound in a local rather than systemic manner, for example, via injection of the compound directly into a tissue, which is preferably a heart muscle tissue or a liver tissue, often in a depot or sustained release formulation.

Furthermore, one may administer the drug in a targeted drug delivery system, for example, in a liposome coated with tissue-specific antibody. The liposomes are targeted to and taken up selectively by the tissue.

In cases of local administration or selective uptake, the effective local concentration of the agent may not be related to plasma concentration.

Thus, the present invention also contemplates a method of gene therapy of a mammal. Such a method utilises a gene therapy construct which includes an isolated polynucleotide comprising a nucleotide sequence encoding at least one SOX protein selected from the group consisting of SOX7, SOX17 and SOX18 or a biologically active fragment thereof, wherein said polynucleotide is ligated into a gene therapy vector which provides one or more regulatory sequences that direct expression of said polynucleotide in said mammal.

Typically, gene therapy vectors are derived from viral DNA sequences such as adenovirus, adeno-associated viruses, herpes-simplex viruses and retroviruses. Suitable gene therapy vectors currently available to the skilled person may be found, for example, in Robbins et al., 1998.

If “anti-sense” therapy is contemplated, then one or more selected portions of a Sox7, Sox17 and/or Sox18 nucleic acid may be oriented 3′→5′ in the gene therapy vector.

Administration of the gene therapy construct to said mammal, preferably a human, may include delivery via direct oral intake, systemic injection, or delivery to selected tissue(s) or cells, or indirectly via delivery to cells isolated from the mammal or a compatible donor. An example of the latter approach would be stem-cell therapy, wherein isolated stem cells having potential for growth and differentiation are transfected with the vector comprising a Sox7, Sox17 and/or Sox18 nucleic acid. The stem-cells are cultured for a period and then transferred to the mammal being treated.

Delivery of said gene therapy construct to cells or tissues of said mammal or said compatible donor may be facilitated by microprojectile bombardment, liposome mediated transfection (e.g., lipofectin or lipofectamine), electroporation, calcium phosphate or DEAE-dextran-mediated transfection, for example. A discussion of suitable delivery methods may be found in Chapter 9 of CURRENT PROTOCOLS IN MOLECULAR BIOLOGY (Eds. Ausubel et al.; John Wiley & Sons Inc., 1997 Edition), for example, which is herein incorporated by reference.

For example, a nucleic acid encoding may be introduced into a cell to enhance the ability of that cell to promote cell differentiation, conversely, Sox18 antisense sequences such as 3′→5′ oligonucleotides may be introduced to decrease tumorigenesis in that cell.

In an alternate embodiment, a polynucleotide encoding a modulatory agent of the invention may be used as a therapeutic or prophylactic composition in the form of a “naked DNA” composition as is known in the art. For example, an expression vector comprising said polynucleotide operably linked to a regulatory polynucleotide (e.g. a promoter, transcriptional terminator, enhancer etc) may be introduced into an animal, preferably a mammal, where it causes production of a modulatory agent in vivo, preferably in a heart muscle tissue or a liver tissue.

The step of introducing the expression vector into a target cell or tissue will differ depending on the intended use and species, and can involve one or more of non-viral and viral vectors, cationic liposomes, retroviruses, and adenoviruses such as, for example, described in Mulligan, R. C., (1993). Such methods can include, for example:

A. Local application of the expression vector by injection (Wolff et al., 1990), surgical implantation, instillation or any other means. This method can also be used in combination with local application by injection, surgical implantation, instillation or any other means, of cells responsive to the protein encoded by the expression vector so as to increase the effectiveness of that treatment. This method can also be used in combination with local application by injection, surgical implantation, instillation or any other means, of another factor or factors required for the activity of said protein.

B. General systemic delivery by injection of DNA, (Calabretta et al., 1993), or RNA, alone or in combination with liposomes (Zhu et al., 1993), viral capsids or nanoparticles (Bertling et al., 1991) or any other mediator of delivery. Improved targeting might be achieved by linking the polynucleotide/expression vector to a targeting molecule (the so-called “magic bullet” approach employing, for example, an antigen-binding molecule), or by local application by injection, surgical implantation or any other means, of another factor or factors required for the activity of the protein encoded by said expression vector, or of cells responsive to said protein.

C. Injection or implantation or delivery by any means, of cells that have been modified ex vivo by transfection (for example, in the presence of calcium phosphate: Chen et al., 1987, or of cationic lipids and polyamines: Rose et al., 1991), infection, injection, electroporation (Shigekawa et al., 1988) or any other way so as to increase the expression of said polynucleotide in those cells. The modification can be mediated by plasmid, bacteriophage, cosmid, viral (such as adenoviral or retroviral; Mulligan, 1993; Miller, 1992; Salmons et al., 1993) or other vectors, or other agents of modification such as liposomes (Zhu et al., 1993), viral capsids or nanoparticles (Bertling et al., 1991), or any other mediator of modification. The use of cells as a delivery vehicle for genes or gene products has been described by Barr et al., 1991 and by Dhawan et al., 1991. Treated cells can be delivered in combination with any nutrient, growth factor, matrix or other agent that will promote their survival in the treated subject.

In order that the invention may be readily understood and put into practical effect, particular preferred embodiments will now be described by way of the following non-limiting example.

### EXAMPLES

### Example 1

Treatment of Artherosclerosis, Restenosis using Sox18

Proliferation, abnormal growth and migration of smooth muscle cells leads to intimal hyperplasia and plays a major role in a number of cardiovascular disorders, including atherosclerosis. This characteristic clinical injury is also the basis for “restenosis” or re-blockage of arteries after clogged or stenotic arteries are mechanically dilated with a balloon on a catheter to restore circulation in arteries (i.e., balloon angioplasty). This localised proliferation of smooth muscle cells impinges on the arterial “endothelial cell-populated” lumen and circulatory blood flow.

The expression of Sox18 in endothelial cells (during mouse embryogenesis at 8.5 to 9.5 d.p.c. between the nascent somites and in the adult) and smooth muscle; and its ability to promote differentiation indicates that Sox18 can be used for gene therapy applications in a number of occlusive cardiovascular disorders.

Sox18 delivered by two-balloon catheters via direct gene transfer or liposome mediated techniques and expressed by plasmid or viral vectors can prove useful in regulating smooth muscle proliferation after angioplasty or arterial injury and stimulating/accelerating cell re-population to resurface the arterial lumen which could reduce vasoconstriction and thrombogenesis (the methodologies pertaining to these techniques, i.e., two-balloon catheters via direct gene transfer or liposome mediated techniques and expressed by plasmid or viral vectors, are described in detail in articles by Nabel, R. et al. (1990 and 1993) and Ohno, T. et al. (1994).

For example, direct intra-arterial gene transfer of Sox18 can be performed according to the following procedure:

A double balloon intravascular catheter (CR Baid Inc., Billerica, Mass.) is inserted into the iliofemoral arteries after anaesthetisation and sterile surgical exposure of the artery. Both balloons are inflated, and the segment is irrigated with 5 mL heparinised saline and 5 mL Opti-MEM™ (Gibco BRL) to rinse blood from the vessel. The arterial segmented is partially denuded by inflation and passage of the proximal balloon. Liposomes containing 30 mg of DNA comprising a Sox18 expression vector are mixed with liposomes (DOTMA and DOPE, BRL) and instilled into the arterial segment between the two balloons at 150 mm Hg in the left and right iliofemoral arteries and incubated for 20 min.

Alternatively, the Sox18 gene could be introduced directly with a replication defective Sox18 retroviral vector. A Sox18 transducing Moloney murine leukaemia virus vector, prepared from yCRIP cells as, for example, described by O'Danos et al. (1988) is used to generate viral particles which are filtered and concentrated by centrifugation as, for example, described by Price et al. (1987). The viral supernatant is instilled for 30 min in the central space of the catheter, with polybrene (6 mg/mL) added after introduction of the virus. The catheter is then removed and antegrade blood flow is restored.

This technology has delivered the herpes virus thymidine kinase gene and the retinoblastoma gene product (a cell cycle regulator), separately simultaneous with balloon angioplasty to reduce smooth muscle cell proliferation and neointima formation in rat and porcine femoral artery models of restenosis (Ohno, T. et al., 1994, supra; Chang, M W et al., 1995).

Direct introduction of p21 with adenoviral vectors into malignant cells, completely suppresses growth in vitro and in vivo (Yang, Z Y et al., 1995). Since SOX18 is a regulator of this potent cell cycle inhibitor, Sox18 can also be used for treatment of restenosis using a similar approach to that described above.

### Example 2

Treatment of Pulmonary Diseases using Sox18

Sox18 could be delivered and expressed in the pulmonary vasculature/endothelium and lung parenchyma for the treatment of pulmonary diseases such as, for example, pulmonary fibrosis/thrombosis by delivery into the pulmonary artery of viral/non-viral vectors, cationic liposomes and/or adenoviral vectors comprising Sox18 using percutaneous right heart catheterisation such as, for example, described by Muller, D W M et al. (1994).

For example, the species/animal is sedated, intubated, and anaesthetised. With the use of sterile techniques, percutaneous catheterisation is performed through the right femoral vein. An 8F introducer sheath is placed in the right femoral vein by using the seldinger technique and a 7F end hole balloon tipped catheter (Meditech) is inserted through the sheath inflated with 1 cm³ of air and advanced through the inferior vena cava, right atrium, and right ventricle into the left pulmonary artery under fluoroscopic guidance. The catheter is then floated into the left posterior basal artery and lodged into the pulmonary artery to occlude blood flow. Five mL of contrast is subsequently delivered through the catheter to confirm the catheter position and the arrest of blood flow. Confirmation of the arrest of blood flow is performed and delivery of DNA and vectors is allowed subsequently to proceed antegradely in the artery and not allowed to diffuse retrogradely. The artery is flushed with 20-mL sterile saline through the end hole of the catheter. The Sox18 DNA expression vector and liposomes are prepared 15 minutes before transcatheter injection by dilution of 5 mg DNA in 0.5 mL Opti-MEM™ (Gibco BRL) and 10 mg Lipofectamine (2,3-dioleyloxy-N-[2(sperminecarboxamido) ethyl]-N, N-dimethyl-1-propanaminium trifluroactetate and diolecylphosphatidylethanolamine) in 0.5 mL of Opti-MEM™. The DNA and liposome solution are then mixed by vortexing and diluted in Opti-MEM to a final volume of 1.6 mL.

If Sox18 is introduced in the form of an adenoviral vector, viral stocks of 1.2×10⁹ pfu/mL, are prepared by diluting 1 mL of virus lysate in 0.6 mL PBS to a total volume of 1.6 mL. The plasmid/liposome mix or the viral stock of the Sox18 expression vectors are infused through the distal end of the balloon into the left posterior basal artery, flushed with 1.0 mL of Opti-MEM™ to clear the dead space of the catheter and incubated for 20 rain. After incubation, the balloon is deflated, the catheter and introducer sheath removed and the right femoral vein is compressed to obtain haemostasis. This technique ensures deliver to the pulmonary vasculature and the alveolar septa. (see Muller et al., 1994, supra).

### Example 3

Neovascularisation/Treatment of Ischaemic Heart Injury, Artherosclerotic Plaques etc using Sox18

Delivery of recombinant Sox18 into arterial walls can also have utility in the stimulation of vascular smooth muscle cells to improve blood supply and flow in a several cardiovascular disorders including ischaemic heart injury and the neo-vascularisation of atherosclerotic plaques. This can be achieved using a similar double balloon intravascular catheter mediated gene transfer approach to that described above for Example 3. In support of this potential, double balloon intravascular catheter mediated gene transfer of FGF- 1 (Nabel, E.G. et al., 1993) and PDGF (Pompili, V. J. et al., 1995) into the femoral arteries has resulted in induced intimal hyperplasia, angiogenesis and matrix deposition.

Thus, Sox18 can be used to induce vascularisation of skeletal muscle tissue that is used to heal/patch weakened myocardial wells in cardiac injury patients.

### Example 4

Growth and Proliferation of Vascular Endothelial Cells using Sox18

Delivery of recombinant Sox18 into arterial walls during angioplasty can also have utility in the growth/proliferation of vascular endothelial cells that are denuded from the vessels by this process. This would aid in the generation of paracrine inhibitors of vascular smooth muscle migration and proliferation. Introduction of endothelial cell products after balloon injury has substantially inhibited cell proliferation, migration, matrix formation required for neointima formation (von der Leyen et al., 1995).

### Example 5

Tissue Engineering using Sox18

Sox18 can also have utility in the design and construction of artificial blood vessel and vascular grafts. Sox18 could be used to selectively support/promote/induce endothelial attachment to synthetic polymers that would aid in hemocompatibility. Artificial blood vessel and vascular grafts have been disclosed, for example, by Langer, R. and Vacanti, J. (1993).

Furthermore, Sox18 can have utility in inducing endothelialisation of vascular. grafts in vivo mediated by gene therapy protocols.

### Example 6

Treatment of Tumours

By way of background, solid tumours rely on blood vessels for growth and spreading by metastasis. Intervention using agents that interfere with blood vessel development holds great promise for therapy of a wide range of cancers. Blood vessels play an essential role in maintaining the supply of nutrients to every organ and tissue in the body. The formation of blood vessels de novo during embryo development is known as vasculogenesis, and involves the differentiation of endothelial cells from mesenchymal precursors and their organisation into tubular structures, the primary blood vessels. The development of capillary networks through sprouting of existing vessels is known as angiogenesis, and predominantly occurs in the embryo. The blood vessels form a stable network that in adults is usually modified only in response to injury or cyclic physiological changes.

Angiogenesis begins with the degradation of the basement membrane by proteases secreted by activated endothelial cells that migrate and proliferate, leading to the formation of solid endothelial cell sprouts into the stromal space. Vascular loops are then formed and capillary tubes develop with formation of tight junctions and deposition of new basement membrane.

Tumour Angiogenesis

During the development of solid tumours, angiogenesis can be characterised by two distinct stages. The prevascular stage is characterised by a tumour mass of 1-2 mm, which is separated from the vascular network and obtains nutrients and disposes of waste via diffusion. The prevascular stage is prolonged, occurring over several years in the case of breast cancer. The vascular stage is characterised by rapid tumour growth, expansion, invasion and metastasis. It is this stage at which cancers can become life threatening.

Several factors are necessary for the cascade of angiogenesis in tumours:

1. An increase in tumour cell number leading to increased metabolic demands and local hypoxia;

2. Increased expression of vascular growth factors, eg. vascular endothelial growth factor (VEGF), an endothelial cell mitogen;

3. Breakdown of the basement membrane to provide an avenue for blood vessel sprouting and invasion of tumour cells into the circulatory system.

So critical is the degree of vascularisation to tumour growth, survival and metastasis that this parameter has been used as an accurate prognostic indicator in ovarian cancer (Hollingsworth, H. et al, 1995; Gasparini, G. et al, 1996).

Control of Angiogenesis as a Cancer Therapy

Over the last decade, unprecedented interest has arisen in the prospect of cancer therapy by manipulating angiogenesis in the patient. In this regard, systemic administration of angiostatin has been shown to potently inhibit the growth of primary carcinomas in mice (O'Reilly, M. S., et al, 1996). An almost complete inhibition of tumour growth was observed without detectable toxicity or resistance. The carcinomas regressed to microscopic dormant foci in which tumour cell proliferation was balanced by apoptosis in the presence of blocked angiogenesis. Significantly, regression of primary tumours was not accompanied by toxicity. These data have recently been substantiated in clinical trials.

Preclinical data have also come from trials of the protein inhibitor endostatin. Like angiostatin, endostatin was shown to repeatedly shrink tumours; each round of shrinkage occurred at the same rate, suggesting that the endothelial cells do not become drug resistant. After 2 to 6 repetitions, depending on the cancer type, the cancers failed to recur (Boehm, T. et al,).

Clearly this strategy is a potentially powerful one in tumour management. Unique features of these treatments are the lack of side effects and of cumulative drug resistance. However, a significant weakness with agents such as endostatin and angiostatin is that the precise mechanism of action is unknown, which makes clinical approval and the design of analogues more challenging. Further research is needed into the basic molecular mechanisms of angiogenesis and new possible entry points for therapeutic intervention of tumour angiogenesis.

Treatment of Tumorigenesis using Sox 18

The inventors hypothesise that Sox18 as well as other subgroup F Sox gene such as Sox7 and Sox17, are important for the control of angiogenesis/vasculogenesis. Accordingly, it is believed that disruption of subgroup F Sox gene function leads to impaired vascularisation of tumours and hence retardation of their growth.

Pilot Study to Assess the Effect of Subgroup F Sox Antisense Oligonucleotides on Tumour Progression in Mice.

The inventors carry out a pilot study for management of tumour growth by direct suppression of Sox7, Sox17 and/or Sox18 activity in vivo. These studies employ a mouse tumour model as described above. Antisense oligonucleotides are injected directly into established tumours and the effect on tumour progression monitored. A precedent for such a study has been described (Stein, C. A. et al, 1993).

Substantial progress has been made in the development of oligonucleotides for therapeutic use. In particular, phosphorothioate-substituted oligonucleotides (PS oligonucleotides) have emerged as an attractive therapeutic agent, combining ease of bulk synthesis, stability in vivo due to nuclease-resistance, ability to be transported into target cells, and a high rate of retention in target cells and the body as a whole (Stein, C. A. et al, 1993). Stability is enhanced when PS substitutions are introduced at all phosphate positions in the oligo (D. Skingle, Pacific Oligos, pers. comm.). When PS oligonucleotides are administered in mice either intraperitoneally (IP) or intravenously (IV), their half-life of transportation from blood plasma to surrounding tissue is about 20 minutes, whereas their half-life in clearance from the body is some 34 hours (Stein, C. A. et al, 1993). IV injection is ideal for the proposed study since the target cells are in direct contact with the bloodstream.

Accordingly, Sox7, Sox17 and/or Sox18 antisense PS oligonucleotides are synthesised spanning their respective translation initiation sites. Tumours are induced by injection of melanoma cells into mice as described above. Once tumours are established (7 days after injection), mice are injected with 3 IV or IP doses of 40 (g of antisense oligo per gram of body weight, at 3-day intervals, as described (Kitajima, I. et al, 1992). Mice injected with sense oligonucleotide or saline serve as controls. Tumours are removed, cleaned and weighed 4, 8, 15 and 60 days after commencement of each treatment. At least 3 mice are analysed for each timepoint, and mean tumour weights in treated and untreated animals are compared by statistical analyses of variance and student's T test. Results are correlated to Sox7, Sox17 and/or Sox18 expression by Western blot analysis of SOX7, SOX17 and/or SOX18 levels in excised tumours.

Similar studies have been reported in which fibrosarcomas induced by injection of HTLV-I Tax-transformed fibroblasts in mice were suppressed by IP injection of antisense NF-κB PS oligonucleotides (Kitajima, I. et al, 1992). In this study, tumours almost completely regressed by 15 days of treatment, and did not return by 5 months after treatment. In contrast, untreated or sense-treated mice died between 8 and 12 weeks.

The inventors proposed pilot study involving Sox7, Sox17 and/or Sox18 support and extend the approaches described herein for establishing a role for Sox18 and for Sox7, Sox17 in tumour vasculogenesis and growth. The pilot study also serves as a basis for further studies in which the role of this gene in tumour metastasis can be explored more fully. Further, it provides preliminary data on which to base therapeutic approaches that target the Sox7, Sox17 and/or Sox18 pathway of endothelial cell stimulation.

### Example 7

Sox18 Mutations Underlie Cardiovascular and Hair Follicle Defects in RAGGED MICE

By way of background, ragged (Ra) is a semi-dominant mutation that arose spontaneously in a crossbred stock of mice (Carter, T. C. et al., 1954). Ra heterozygotes are viable and healthy with thin, ragged coats comprised of guard hairs and awls but lacking auchenes and zigzags (Carter, T. C. et al., 1954). Homozygotes almost completely lack vibrissae and coat hairs, display generalised oedema and cyanosis, rarely survive past weaning and, depending on the genetic background, may have an accumulation of chyle in the peritoneum (Carter, T. C. et al., 1954; Herbertson, B. M. et al., 1964; Wallace, M. E. 1979). Two other alleles of Ra have been reported: Ra^{j} , with a phenotype indistinguishable from Ra, and opossum (Ra^{Op}) (ref. 8,9). Ra^{Op} heterozygotes have a phenotype similar to that of Ra homozygotes, whilst Ra^{Op} homozygotes die by 11.5 days post coitum (dpc) (Green, E. L. et al, 1961; Mann, S. J., 1963). Detailed histological study of Ra homozygous embryos has revealed that the oedema is due to dysfunction of the cardiovascular system, typified by superficial haematomata and dilation, distension or rupture of peripheral blood vessels (Slee, J. 1957²) Coat defects are due to a reduction in the total number of hair follicles, with the later-forming follicles the most affected (Slee, J. 1957). Some follicles do form, but many show arrested development and are not associated with hair eruption (Slee, J. 1957).

Methods

In situ hybridisation.

We staged embryos of the outbred mouse strain CD1 by established morphological criteria (Theiler, K. 1972). Standard in situ hybridisation protocols (Christiansen, J. H. et al, 1995) were used. The Flk1, Ptc1 and Shh riboprobes have been described elsewhere (Yamaguchi, T. P. et al., 1993; Hahn, H. et al., 1996; Roelink, H. et al., 1995). The Sox18 in situ probe was transcribed from a Pst1/Xba1 cDNA fragment spanning a portion of the trans-activation domain and 3′ UTR (coding nucleotides 818-1673). Section data were generated by embedding embryos after whole-mount in situ hybridisation in paraffin and cutting 7-10 μM sections. Sections were collected on pre-cleaned slides (SuperFrost™, Menzel-Gläser), de-waxed in two changes of xylene and mounted in a xylene-compatible mountant (Mount-Quick, Daido Sangyo) and were viewed and photographed using Nomarski optics. The Flk1 and Flt1 knock-out embryos were provided by Janet Rossant and Andras Nagy, and have been described elsewhere (Shalaby, F. et al., 1995; Fong, G. H., et al., 1995).

Interspecific Backcross.

The interspecific backcross has been described previously (Abbott, C. et al., 1994). Briefly, Ra heterozygous female mice were crossed with Mus spretus males. F₁ female mice with the Ra phenotype were then crossed to C3H/HeH males. A 201 bp fragment of genomic DNA (between 1296 and 1097 bp upstream of the Sox18 initiator codon) was amplified using the PCR with primers 5′-ACCAATGACCCATGCTCCAG-3′ [SEQ ID NO: 40] and 5′- GCAGGCAGTAAT GTGGACA-3′ [SEQ ID NO: 41]. An HaeIII restriction site polymorphism in this region (present in Mus musculus but absent from M. spretus) was used to distinguish between PCR products from the Sox18 alleles of the two species.

DNA amplification and sequencing. Genomic DNAs were obtained from the Jackson Laboratory or extracted directly from Ra mouse tissue. The Sox18 open-reading frame and sole intron were amplified using PCR with either Taq or Pfu DNA polymerase. PCR products were sequenced after subcloning into pBSII KS+ or directly after purification on Centricon 100 columns (Amicon) according to the manufacturer's specifications. Sequencing was performed using an ABI Prism dye terminator system. Cycling was conducted in a DNA Thermal Cycler (Perkin Elmer Cetus) thus; 95° C. for 5 min; 25 cycles of 96° C. for 30 sec, 50° C. for 15 sec and 60° C. for 4 min. Various combinations of the following forward and reverse primers were used for the amplification and sequencing of Sox18.

Forward primers: Primer A 5′-TCCAAAGCCGCTGCCCTCTC CCATCATTA-3′ [SEQ ID NO: 42],
Primer B 5′-GCGGAATTCAGACCTA GCCCACACCAGCAGTCC-3′ [SEQ ID NO: 43],
Primer C 5′-GCGGAATTCACCA TGGGGGGCTCTGCGCTGGGG-3′ [SEQ ID NO: 44],
Primer D 5′-GCGAATTCACC ATGCAGAGATCGCCGCCCGGCTACG-3′ [SEQ ID NO: 45],
Primer E 5′-CAAAG CGTGGAAGGAGCTGAAC-3′ [SEQ ID NO: 46], Primer
F 5′-GCGAATTCCTGGG AGCCGGGTCTCTTGGTC-3′ [SEQ ID NO: 47], Primer N 5′-
GCGAATTCACCGGGAC CCGAGCTTCTGCTACG-3′ [SEQ ID NO: 48]. Reverse
primers: Primer G 5′-CAGAA TTCACCGTCGGCAGTTTGGCGCTCTCC-3′ [SEQ ID NO: 49],
Primer H 5′-GCGGAA TTCCTGTAGGCGAAGGGAGCCTG-3′ [SEQ ID NO: 50],
Primer I 5′-GCGAATT CTTATTTAGCTCCAGCCTCCGGACC-3′ [SEQ ID NO: 51],
Primer J 5′-CAGAGTG GGTAGCTCACGGAAG-3′ [SEQ ID NO: 52], Primer K 5′-
GCGAATTCA GCAGAAGCTCGGGTCCCGTGC-3′ [SEQ ID NO: 53], Primer L 5′-
GCGAATTCTTA TCTAGCCTGAGATGCAAGC-3′ [SEQ ID NO: 54], Primer M 5′-
TAGOCCACCA GCTCTAAAGGCTGTTGCATA-3′ [SEQ ID NO: 55],

analysed with Sequencher 3.0 software (Gene Codes). Mutations were confirmed by at least four independent rounds of PCR amplification and sequencing using DNA samples from at least two different animals of each mutant stock.

Trans-activation Assay.

The portion of Sox18 encoding the trans-activation domain (amino acids 253-345) was amplified directly from genomic DNAs using Pfu DNA polymerase with the primers 5′-GCGAATTCAGCAGAAGCTCGGGTCCCGTGC-3′ [SEQ ID NO: 56] and 5′-GCGAATTCCTGGAGCCGGGTCTCTTGGTC-3′ [SEQ ID NO: 57]. PCR products were digested with EcoRI and sub-cloned into the EcoRI site of the expression vector pGAL0. Cloning junctions were sequenced to ensure clones were in the correct frame. Transient transfections, CAT assays and thin layer chromatography were performed in triplicate as described previously (Hosking, B. M. et al., 1995).

Results

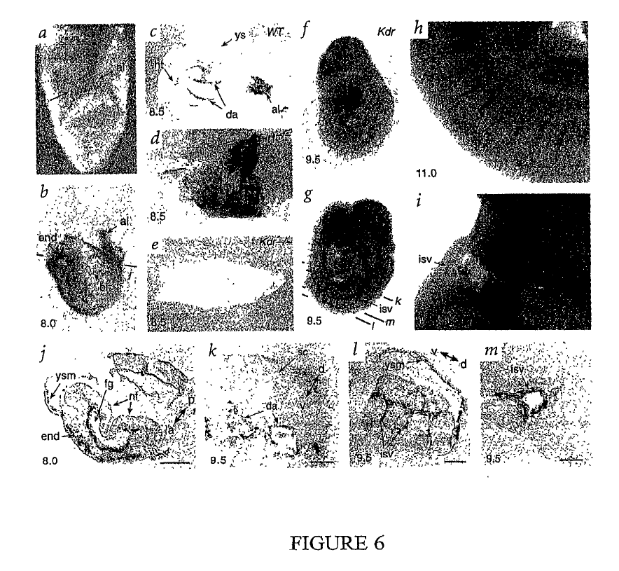

As a starting point for examining the relationship between Sox18 and ragged, the present inventors examined the expression of Sox18 mRNA during mouse embryo development by in situ hybridisation. Expression was first detected in the allantois and yolk sac blood islands at 7.5 dpc and persisted at these sites until 8.5 dpc (FIGS. 6a-c). At 8.0 dpc, expression was seen in the embryo proper, including cells fated to become the endocardium (FIG. 6b). At 8.5 dpc Sox18 expression was detected in the paired dorsal aortae and the heart (FIG. 6c). In addition to expression in vessels formed by vasculogenesis, such as the dorsal aortae, Sox18 expression was associated with those formed by angiogenesis, such as the intersomitic vessels (FIGS. 6f, h). Sox18 showed a spatial and temporal expression pattern strikingly similar to the early endothelial marker Flk1 at 9.5 dpc (FIGS. 6f, g). Sox18 expression was completely lacking in Flk1^{-/-} embryos, in which endothelial cell differentiation is blocked at an early stage (Shalaby, F. et al., 1995) (FIG. 6e), but occurs in Flt1^{-/-} embryos, in which endothelial cells differentiate but fail to organise into functional vessels (Fong, G. H. et al., 1995) (FIG. 6d). The lack of expression of Sox18 in Flk1-deficient embryos, along with the onset of Sox18 expression from as early as 7.5 dpc, indicate that Sox18 is associated with vascular endothelial development from the earliest stages.

From the foregoing, the inventors concluded that Sox18 expression is transient in the nascent vessels in all cases, consistent with a role for Sox18 in cell differentiation. Blood vessels at these stages of embryogenesis consist entirely of endothelial cells, and the data suggest that transient Sox18 expression is associated with endothelial cell differentiation and/or stimulation.

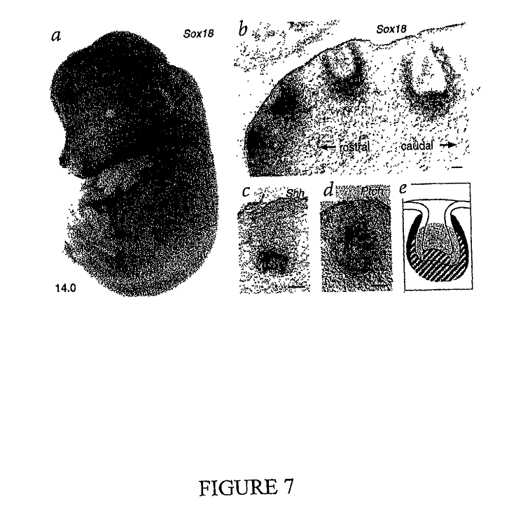

From 12.5 dpc Sox18 expression was observed in the developing vibrissae (sensory) follicles (FIGS. 6i, 7a) and persisted there until about 15.0 dpc (data not shown). Expression was observed in the first wave of pelage hair follicles at 14.0 dpc (FIG. 7a) and in nascent follicles in subsequent waves during embryogenesis (data not shown). Tissue sections revealed that Sox18 is expressed in the mesenchyme subjacent to the epithelial follicle placode, and persists in the mesenchymal cells surrounding the invaginating epithelium to the dermal papilla stage (FIGS. 7b, e). The mesodermal expression of Sox18 in the hair follicle contrasts with the expression of Shh (FIGS. 7c, e), which marks the invaginating epithelium of the developing follicle (Iseki, S. et al, 1996) and its receptor, Ptc1 (FIGS. 7d, e), which is expressed more diffusely in the developing follicle (St Jacques, B. et al, 1998). The transient expression of Sox18 in nascent blood vessels and hair follicles is reminiscent of the expression of Sox9 during chondrogenesis (Wright, E. et al., 1995; Bi, W. et al., 1999) and Sry during testis determination (Koopman, P. et al., 1990).

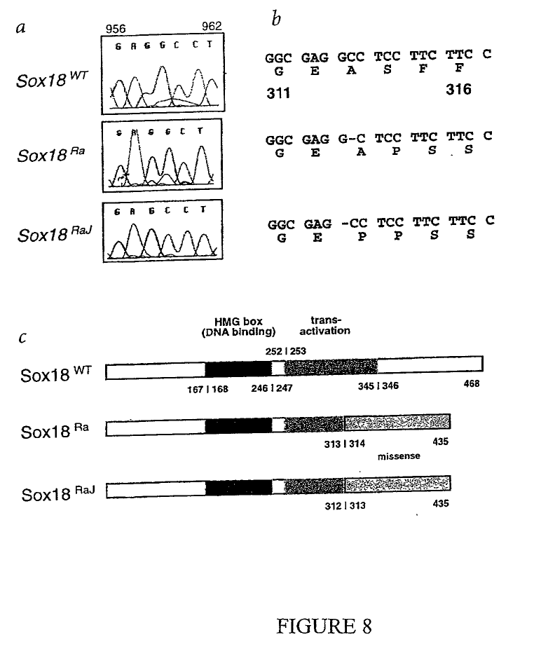

The inventors tested the genetic linkage between Sox18 and Ra in a mouse interspecific backcross segregating for the Ra phenotype, using a polymorphism between the Mus musculus and M. spretus Sox18 alleles. No recombinants between Sox18 and Ra were found among the 490 Ra/+ backcross mice tested (data not shown), indicating that the two loci are genetically inseparable. Sox18 from the Ra and Ra^{J} mice (SOX18^{Ra} and Sox18^{RaJ} respectively) was then screened for mutations by polymerase chain reaction (PCR) and sequencing. A cytosine residue at coding nucleotide position 960 was deleted in Sox18^{Ra} (FIGS. 8a, b). This mutation introduces a frameshift leading to missense coding for 122 amino acids and premature truncation of the SOX18 protein (FIG. 8c). Sequencing of Sox18^{RaJ} revealed that a guanine residue at coding nucleotide position 959 was deleted (FIGS. 8a, b). This mutation introduces a frameshift and missense coding for 123 amino acids and premature stop similar to that in Sox18^{Ra}. No further mutations were found in the open reading frame (ORF) of either Sox18^{Ra} or Sox18^{RaJ} and only silent polymorphisms were detected in Sox18 between the wild-type background strains C3H/HeSnJ, C57BL/6 and DBA/2J (data not shown). It remains to be determined whether mutations affect the structure, or indeed the regulation, of Sox18 in Sox18^{RaOp}; while mating studies have suggested that Ra and Ra^{Op} are allelic (Mann, S. J. 1963), it is formally possible that Ra^{Op} is a mutation in a closely linked gene acting in the same developmental pathway. The present data clearly identify Sox18 as the Ra gene.

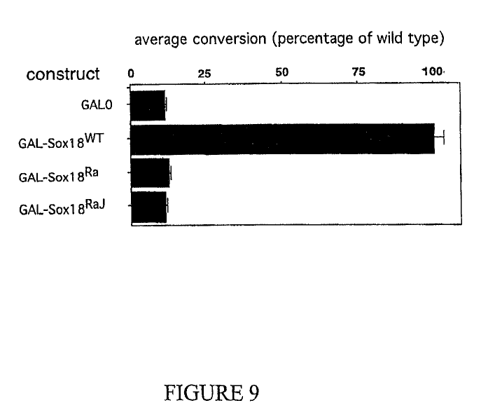

SOX18 has been shown to be a potent activator of transcription in a cell culture assay (Hosking, B. M. et al., 1995). Mutations in Sox18^{Ra} and Sox18^{RaJ} would be expected to truncate the trans-activation domains of the encoded proteins (FIG. 8c). To examine the biochemical consequences of these mutations, we tested the ability of the mutant proteins to activate transcription in a GAL4 assay. Fusion proteins expressed from clones pGAL-SOX18^{Ra} and pGAL-SOX18 , containing the SOX18 and SOX18 trans-activation domains respectively, failed to activate transcription above basal levels, while fusion protein from clone pGAL-SOX18 , containing the trans-activation domain of SOX18 from wild-type mice (SOX18 ), activated strongly (FIG. 9). These data indicate that SOX18 proteins from both Ra and Ra mice have lost their ability to function as activators of transcription.

Ra and Ra^{J} are semidominant mutations, with heterozygotes showing an intermediate phenotype between homozygous mutant and wild-type mice. The heterozygous phenotype may be due to haploinsufficiency for Sox18, as has been described for mutations in other Sox genes (Pingault, V. et al., 1998; Wagner, T. et al., 1994). Alternatively, since the Sox18 proteins from Ra and Ra^{J} contain the DNA-binding domain but not an active trans-activation domain, mutant proteins may act in a dominant-negative manner. Defective vasculogenesis and hair follicle development in Ra and Ra^{J} mice are likely to result from failure of SOX18 to activate target genes in these developmental pathways. The identification of these target genes furthers our understanding of both cardiovascular and hair follicle development.

In view of the above, the present inventors have used in vitro and in vivo protein-protein interaction assays to screen for interaction with a number of transcriptional factors and cofactors that have been implicated in vasculogenesis and angiogenesis, and that are directly involved in mediating the transcriptional effects of Sox18. In this way the inventors have already identified the MADS box transcription factor, MEF2C, as a putative interacting partner protein for SOX18 during vascular development. It has been demonstrated MEF2C is expressed in developing endothelial cells and smooth muscle cells, as well as the surrounding mesenchyme, during embryogenesis. Targeted deletion of the mouse MEF2C gene resulted in severe vascular abnormalities and lethality in homozygous mutants by embryonic day 9.5. Endothelial cells were present and were able to differentiate, but failed to organise normally into a vascular plexus, and smooth muscle cells did not differentiate in mutant embryos. These vascular defects resemble those in mice lacking VEGF or its receptor Flt-1 (6).

The present inventors have also found that mutant SOX18 proteins produced by Ra, RaJ, RaOp, and Ragl mice do not interact with MEF2C in an in vitro GST-pulldown assay. This last finding underscores the biological significance of the interaction between SOX18 and MEF2C, and further supports the hypothesis that Sox18 mutations in Ra mice act in a dominant-negative fashion.

### Example 8

Transcriptional Structure/Function Analysis of Sox18

Bacterially expressed and affinity purified SOX18 protein binds in a sequence specific manner to the DNA motif AACAAAG [SEQ ID NO: 58] that is recognised by all known members of the Sox gene family characterised to date. Furthermore, Sox 18 was capable of trans-activating gene expression in an AACAAAG-dependent manner. GAL4 hybrid analysis demonstrated that SOX18 is an efficient trans-activator of gene expression. We localised the activation domain to 93 amino acids between amino acids 252 and 345 in SOX18 (Hosking, B., et al., 1995, supra, denoted aa 160-255 in the publication). The effect of post-translational modification on the activity of transcription factors has been widely reviewed. The most studied of these modifications is phosphorylation. The inventors found that 8-Bromo cAMP and Okadaic Acid, which act on PKC and PKA pathways to increase protein phosphorylation, both dramatically increased the trans-activation by SOX18 (Hosking, B., et al., 1995, supra). Thus the phosphorylation status of SOX18 may play a key regulatory role in the activity of this protein. A computer scan (Signal Scan, ANGIS) of the 93aa activation domain suggests two potential protein kinase C (PKC) phosphorylation sites, at serine residues 277 and 280. A potential casein kinase II phosphorylation site was indicated at serine 280.

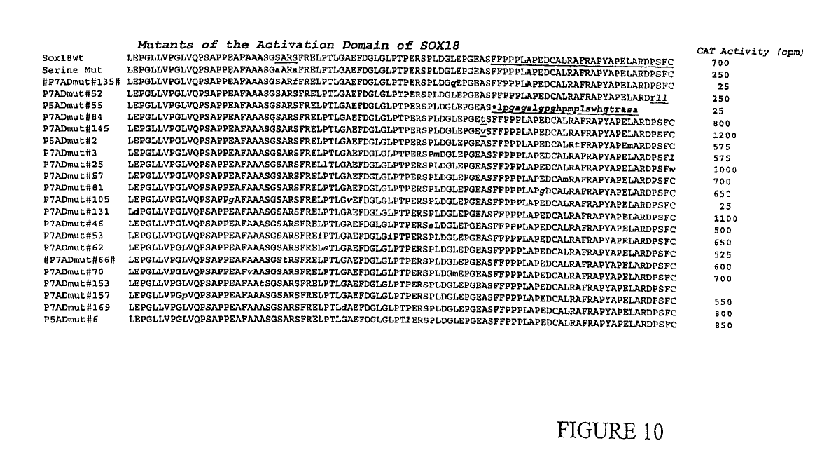

The inventors have also performed rigorous random mutagenesis of the activation domain to identify the critical residues involved in transcriptional activation (FIG. 10). Mutagenesis of the putative PKC site SARS resulted in reduced activity of the trans-activation domain, implicating these sites in SOX18 function (FIG. 10; Serine and #135 Mut, 3-20-fold, respectively). The significance of these potential CKII sites has yet to be determined. Furthermore, mutants, #52 and 55, demonstrated that the carboxy terminus of the SOX18 activation domain was also required for efficient trans-activation and complementation of the GAL4 DBD.

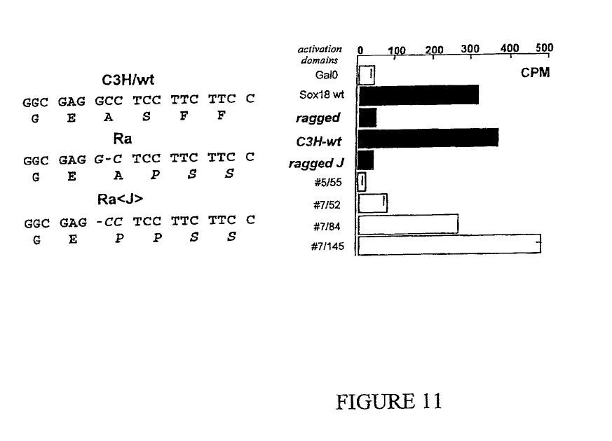

Sequencing of Sox18 DNA from Ra mice detected mutations in this locus suggesting that the locus was responsible for the Ra phenotype. The defect in Ra and the allelic mutant RaJ creates a frameshift in the trans-activation domain (FIG. 11). Fortuitously, the identified mutation was in the identical position as the frame shift mutation (#55) (FIG. 10) that was created by random mutagenesis, and was found to disrupt the ability of the activation domain to complement the GAL4 DBD in the transcription assay. This firmly suggested that the SOX18 protein from the ragged mutant mice was impaired in its ability to trans-activate gene expression.

Verification of this hypothesis was provided by cloning the SOX18 activation domain from the ragged and ragged J mice into the GAL4 hybrid system and demonstrating that its ability to trans-activate gene expression and complement the GAL4 DBD (in contrast to the wild type) was severely impaired (FIG. 11). Furthermore, it demonstrated that the carboxy terminal region of this domain was critical to function. Point mutants, #84 and #145, that changed the EAS sequence to either EtS or EvS had negligible effects on trans-activation by this region, consistent with the notion that the frameshift produced the effect rather than the specific amino acid mutation (FIG. 10). Moreover, in support of this hypothesis, the site-specific mutants, #2, 3, 25, 57 and 81 (FIG. 10) in the C-terminal region of the activation domain, functioned efficiently in the GAL4 hybrid system suggesting that the integrity of the activation domain was not compromised. These expression data raise the possibility of a regulatory relationship between Sox18 and Flk1.

### Example 9

A Expression of Sox18 during Wound Healing

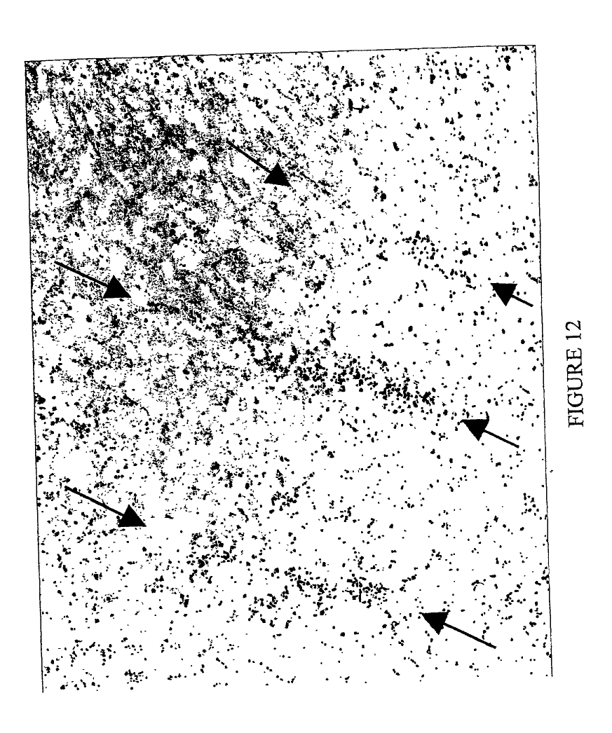

The inventors also examined the expression of Sox18 during wound healing by in situ hybridisation. Full-thickness excisional skin wounds were created in mice and removed during healing. Sox18 mRNA expression was detected in capillaries within the granulation tissue and co-localised with Flk1 and Col4a1 mRNA expression in endothelial cells (FIG. 12). Pre-existing capillaries in the subcutaneous tissue of the surrounding unwounded skin showed no Sox18 expression. Sox18 expression was found early during capillary sprouting in granulation tissue 5d after wounding and persisted throughout healing. These results suggest that Sox18 is involved in the induction of angiogenesis during wound healing and tissue repair, but not the maintenance of endothelial cells in undamaged tissue.

### Example 10

B Sox18 is Mutated in Ragged-like and Opossum Mice

Two further mutations in Sox18 were investigated in the two other known alleles, opossum (Ra^{op}) and ragged-like (Ragl). The Ragl mouse, like the other allelic variants of ragged, arose spontaneously among crossbred strains of mice, and is phenotypically similar to Ra and Rah mice. Heterozygotes of these three alleles are characterised by a thin, ragged coat and oedema at birth caused by vascular dysfunction, while homozygotes are naked and often die in utero or soon after birth (22-25). Ra^{op} represents a more severe class of mutant phenotype, with heterozygotes resembling homozygotes of the other alleles (Green, E. et al, 1961; Mann, S. 1963). Homozygous Ra^{op} animals die in utero at approximately 11.5 days post-coitum, most likely due to cardiovascular dysgenesis.

Methods

Sequencing

The open reading frame of Sox18 in Ragl and Ra^{op} DNA was amplified by PCR, cloned and sequenced as described (Pennisi, D. et al, 2000). The sequence was examined for variations compared to wild-type DNA from background strains of mice (Ra^{op}: C57BL/6J and DBA/2J; Ragl:C57BL/6J Aw-J and 1295). Genomic DNA from background mouse strains and two mice of each mutant stock was obtained from the Jackson Laboratory. To confirm mutations, three rounds of PCR were performed with Taq DNA polymerase (Roche) and one with Hifidelity™ PCR Supermix (Roche), and three clones from each round of PCR were sequenced on both strands.

Transcriptional trans-activation assay

The region of Sox18 downstream from the HMG box (nucleotides 757-1407) was amplified from wild-type and all four ragged mutant DNAs using the primers 5′-CTG GAG CCG GGC CTC TTG CTC-3′ [SEQ ID NO: 59] and 5′-GCG AAT TCT ATC TAG CCT GAG ATG CAA GC-3′ [SEQ ID NO: 60], and Pfu DNA polymerase (Stratagene). These fragments were then cloned into the mammalian expression vector, pGALO, and sequenced as described (Pennisi, D. et al, 2000). The expression constructs were transfected into COS-1 cells and luciferase (LUC) reporter assays were performed in triplicate as described (Chen S. et al, 2000).

Results

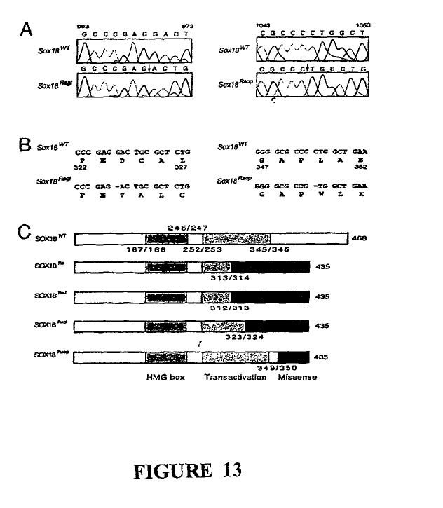

As shown herein, Sox18 is mutated in the Ra and Ra^{J} mice. The single base deletions identified in these alleles, designated Sox18^{Ra} and Sox18^{RaJ}, result in missense coding and truncation of the C-terminus of the SOX18 protein (FIG. 13). In the mutant forms of SOX18 (SOX18^{Ra} and SOX18^{RaJ}), the trans-activation domain is truncated, and its activity in an in vitro assay is completely abolished (Pennisi, D. et al, 2000).

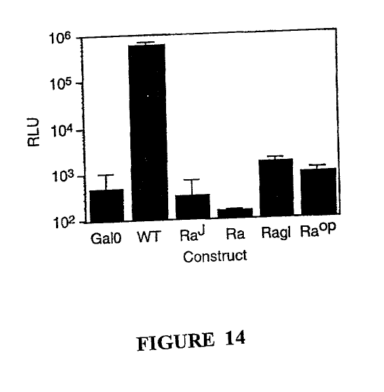

The inventors have also determined the structure of Sox18 in two other known ragged alleles, Ra^{op} and Ragl. Sequencing of Sox18 from DNA of Ragl and Ra^{op} mice revealed single base deletions in both cases (FIG. 13A). As in Sox18^{Ra} and Sox18^{RaJ}, these mutations are predicted to result in missense translation and premature termination of SOX18 at 435 amino acids, compared to the 468 amino acid wild-type protein (FIGS. 13B, C). The deletion of a guanine residue at coding nucleotide 970 in Sox18^{Ragl} causes truncation of the transcriptional activation domain, while the deletion of a cytosine residue at coding nucleotide 1048 in Sox18^{Raop} is beyond the previously determined limit of the trans-activation domain (Hosking, B. et al, 1995). Despite this, the trans-activation ability of both GAL4-SOX18^{Ragl} and GAL4-SOX18^{Raop} fusion proteins was compromised relative to that of wild-type GAL4-SOX18, as measured in a luciferase reporter assay (FIG. 14). It therefore appears that the trans-activation domain of SOX18 is truncated in all four mutant forms of SOX18, suggesting that it extends at least four amino acids further than previously determined (FIG. 13C).

The mutations in all four known allelic variants of ragged lie within a 90 by region of the gene (Sox18^{Ra} and Sox18^{RaJ} coding nucleotides 960 and 959, respectively; FIG. 13C), indicating that this may be a hotspot for mutations in Sox18. In addition, the clustering of mutations in this region provides insights into the molecular function of the SOX18 protein.

It is clear from the data presented herein that the two distinct classes of ragged phenotype correlate with two classes of mutations. Ra^{op} shows the most severe phenotype with missense coding from amino acid 350, while Ra, Ra^{j} and Ragl show a less severe phenotype with missense coding from amino acids 313 (Ra, Ra^{j}) and 324 (Ragl) (FIG. 13C). From the foregoing, it is proposed that a dominant negative mechanism may operate, whereby mutant Sox18 interferes with the function of another protein(s). In support of this possibility, Sox18 null mutant mice have a very mild phenotype, discussed infra, suggestive of functional redundancy, between the highly related Group F SOX proteins SOX7, SOX17 and SOX18. Any dominant negative effect of ragged mutations must be more severe in Ra^{op} than in the other ragged mutants. This situation is reminiscent of thyroid hormone resistance syndromes where the severity of the disease is dependent on the position of the mutation within the ligand-binding domain. In these syndromes the severity of the phenotype increases when more intact protein is present, due to changes in cofactor recruitment and molecular crosstalk (Refetoff, S. et al, 1993). The observations in the present study suggest that a similar mechanism may operate in the case of the SOX18 trans-activation domain, and implicate the 26 amino acid fragment (SEQ ID NO: 37) between the Ragl mutation and the Ra^{op} mutation in recruitment of cofactors by SOX18.

The highly conserved C-terminal region of SOX18 is replaced with missense sequence in all four ragged alleles. The inventors believe that this region of SOX18 is responsible for an important function of the protein that is, as yet, unknown, but is likely to be involved in interaction with other proteins.

### Example 11

C Mice Null for Sox18 are Viable and Display a Mild Coat Defect

The data presented above show that point mutations in Sox18 are the underlying cause of profound cardiovascular and hair follicle defects in ragged (Ra) mice (Pennisi. D et al, 2000). Ra heterozygotes have thin, ragged coats comprised of guard hairs, but lacking the later forming auchenes and zigzags (Carter, T. C. et al, 1954). Ra homozygotes, however, almost completely lack vibrissae and coat hairs, display generalised oedema and cyanosis and rarely survive past weening (Carter, T. C. et al, 1954). In a more severe Ra mutation, Ra^{Op}, heterozygotes typically show a similar phenotype to Ra homozygotes, whereas Ra^{Op} homozygotes die by 11.5 days post coitum (dpc) (Green, E. L. et al 1961; Mann, S. J. 1963). The coat defects in Ra mice are due to a reduction in the total number of hair follicles, with the later forming follicles the most affected (Slee, J. 1957).

In Ra mice, the Sox18 mutations lead to an intact DNA-binding domain but a non-functional trans-activation domain (Pennisi, D. et al 2000). Given the semi-dominant nature of the Ra mutations (Carter, T. C. et al 1954; Green, E. L. et al 1961), it was not clear whether the phenotype of Ra mice was due to haploinsufficiency of Sox18, as has been described for other SOX genes, or whether there was a dominant-negative effect from the mutant Sox18 protein. To address this question, the inventors produced mice null for Sox18.

Materials and Methods

Targeting of Sox18 and Production of chimeric Mice.

Overlapping genomic clones hybridising to Sox18 were obtained from a λ phage library constructed from 129/Sv genomic DNA partially digested with MboI cloned into the BamHI sites of a λ DashII vector (Philippe Soriano, personal communication). Four overlapping clones were mapped using restriction digests and Southern hybridisation. The 1.8 kb, 5′ flanking fragment was generated by high-fidelity PCR from a genomic clone to incorporate XbaI sites and facilitate sub-cloning. The 3′ flanking arm was an 11 kb fragment sub-cloned from a genomic clone. The 3′ and 5′ flanking sequences were cloned into a plasmid, pLoxPneo-1, that contains a neomycin resistance cassette (neo^{r}) driven by the PGK-1 promoter, all flanked by directional LoxP sites (AN, unpublished resource). The LoxP sites were included to allow the excision of intervening sequence in the presence of cre recombinase (Abremski, K et al 1984). A promoterless LacZ reporter cassette (Korn, R et al 1992) was sub-cloned in to the targeting vector so that an in frame SOX18-β-GAL fusion protein would be produced to facilitate gene expression studies. A thymidine kinase cassette (Mansour, S. L. et al 1988) was used in the targeting vector for counterselection in ES cells.

Gene targeting was performed in the RI ES cell line (Nagy, A. et al 1993) using standard protocols (Joyner, A. L. 1993), with the exception that ES cells were grown in the absence of feeder fibroblasts cells on gelatinised plastic tissue culture dishes in media supplemented with LIF and 40 μg of linearised targeting vector was used in each electroporation cuvette. G418-resistant ES cell clones were screened for homologous recombination using genomic Southern blots after HindIII digestion. An external probe was used to detect a native 12 kb band or a band of 8.5 kb in targeted alleles. Two independent ES cell clones were identified as homologous recombinants. Chimeric embryos were produced from them using CD1 donor embryos and the technique of morula aggregation (Wood, S. A. et al 1993), and transferred to pseudopregnant recipients as described by Hogan, B. et al 1994. Germline transmission of the targeted Sox18 allele was achieved with chimaeras produced from the two independently targeted ES cell lines. Analysis of the targeted mutation was conducted on mice and embryos of a 129/CD1 mixed genetic background.

Genotyping of Mice and Embryos.

Mice and embryos used in this study were genotyped by genomic DNA Southern hybridisation (as described above for screening ES cell clones) or by PCR on genomic DNA prepared from ear punches or tail clips as described by Joyner, A. L. et al 1993. To detect the targeted allele, the neo^{r}-specific primers neoR, 5′-CAA GCT CTT CAG CAA TAT CAC G-3′ [SEQ ID NO: 61], and neoF, 5′-ATC TCC TGT CAT CTC ACC TTG C-3′ [SEQ ID NO: 62], were used. To detect the wild-type Sox18 allele, the primers Sox18-Box A, 5′-CCA ACG TCT CGC CCA CCT CG-3′ [SEQ ID NO: 63], and Sox18-Box B, 5′-GCC GCT TCT CCG CCG TGT TC-3′ [SEQ ID NO: 64], were used. Mutant embryos were always genotyped by PCR amplification from a portion of the yolk sac or allantois that had been well rinsed in a large volume of PBS.

RT-PCR Analysis.

cDNA was produced in a reaction containing 1 μg of RNA, 1× “first strand” buffer (Gibco BRL: 50 mM Tris-HCl pH 8.3, 75 mM KCl, 3 mM MgCl2), 375 μM dNTPs, 100 mM DTT, 500 ng pd(N)₆ random primers (Pharmacia), 200 U of M-MLV reverse transcriptase (Gibco BRL) and RNase-free MilliQ™ water in a total volume of 30 μL. The reaction was incubated at 42° C. for 1 hour and 5 μL was used in a 25 μL PCR reaction. The primer pairs used for detection of neo^{r} transcripts were neoR, 5′-CAA GCT CTT CAG CAA TAT CAC G-3′ [SEQ ID NO: 65], and neoF, 5′-ATC TCC TGT CAT CTC ACC TTG C-3′ [SEQ ID NO: 66], for LacZ transcripts, LacZ A, 5′-CAG CAC ATC CCC CTT TCG CC-3′ [SEQ ID NO: 67], and LacZ B, 5′-CCA ACG CAG CAC CAT CAC CG-3′[SEQ ID NO: 68], for the 3′ portion of Sox18 downstream of the LacZ reporter and neo^{r} (encoding trans-activation domain), Sox 18-3′ A, 5′-GGC TTT CCG GGC ACC CTA TG-3′ [SEQ ID NO: 69], and Sox18-3′ B, 5′-AAG CGG TGG AGG GCA AGG AC-3′ [SEQ ID NO: 70], for the region of Sox18 encoding the HMG box, Sox18-Box A, 5′-CCA ACG TCT CGC CCA CCT CG-3′ [SEQ ID NO: 71], and Sox18-Box B, 5′-GCC GCT TCT CCG CCG TGT TC-3′ [SEQ ID NO: 72], for the 5′ region of Sox18 upstream of the LacZ reporter and neo^{r}, Sox18-5′ A, 5′-TGA GAC AGT GGG AGC AGA TGG-3′ [SEQ ID NO: 73], and Sox18-5′ B, 5′-GCA AAG CCA AGT ACG GAG GTC 3′ [SEQ ID NO: 74], and for GAPDH transcripts, GAPDH F, 5′-TCG GTG TGA ACG GAT TTG-3′ [SEQ ID NO: 75], and GAPDH R, 5′-ATT CTC GGC CTT GAC TGT-3′ [SEQ ID NO: 76].

Expression Studies.

Whole-mount immunohistochemistry was done using an anti-PECAM-1 (CD31) antibody (Pharmingen, Calif., USA) at a dilution of 1/250, using standard techniques described elsewhere Wheatley, S. C. et al, 1993.

Analysis of the Coat of Sox18^{-/-} Mice.

The coat of adult Sox18^{-/-} and control mice was analysed by visible appearance for colour, and photographed using standard colour-reversal film. To survey the relative abundance of the various hair types amongst the coat of Sox18^{-/-} and control mice, hairs were plucked in bunches from the mid-dorsum of adult mice, typically three months in age. A broad pair of forceps (Millipore) was used to obtain three samples from each test subject. Using a dissecting microscope, the numbers of each hair type were then counted according to the mouse hair classification of Dry, F. W. 1926. The total number of hairs from each subject varied between 160 and 380 hairs in order to give a statistically valid sampling of hair types. Photography of mouse hairs was performed on a dissecting stereomicroscope (Leica MZ8), using bright field illumination after dry-mounting hairs on a microscope slide with a cover slip and nail polish.

Results

Targeting of the Sox18 Gene.

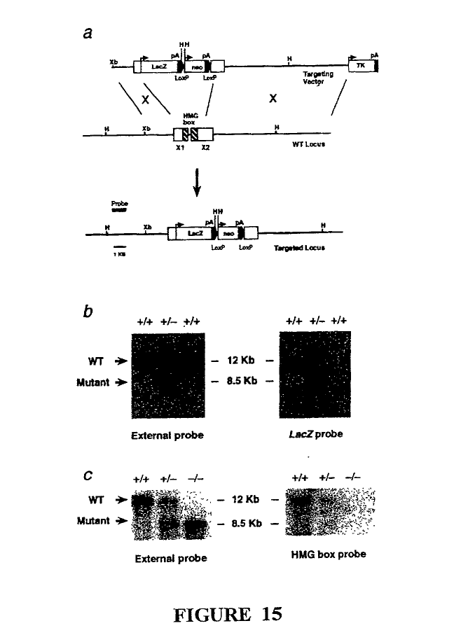

The gene targeting strategy was designed to produce mice null for Sox18 by removing the region encoding the HMG box (DNA-binding domain) and replacing it with a LacZ reporter cassette and a neo^{r} cassette, both of which contain transcription termination sequences. Southern blot analysis of genomic DNA from Sox18^{-/-} and Sox18^{+/-} mice confirmed that the Sox18 locus had been targeted as expected, with the region encoding the HMG box being removed and replaced by the LacZ and neo^{r} cassettes (FIG. 15). However, analysis of Sox18^{-/-} and Sox18^{+/-} embryos at 9.5dpc, a stage when Sox18 is expressed strongly in the developing vascular system Pennisi, D. J. et al, 2000, failed to show β-galactosidase staining (data not shown). Sequencing of the targeting vector confirmed that an inframe fusion protein should have been produced comprising the N-terminal 89 amino acids of SOX18 and β-galactosidase (data not shown). It is unclear why the LacZ reporter cassette is non-functional in Sox18^{-/-} and Sox18^{+/-} embryos, though one possibility is that the N-terminal 89 amino acids of SOX18 has interfered with the enzymatic activity of β-galactosidase.

RT-PCR Analysis of the Mutant Transcript.

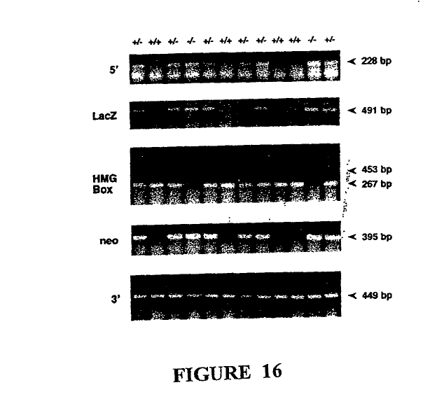

To analyse what transcripts were produced from the targeted Sox18 locus, RT-PCR was conducted on RNA from mutant and wild-type embryos. RT-PCR analysis confirmed that there were no transcripts from the HMG box domain-encoding region from the targeted allele (FIG. 16). As can be seen in FIG. 2, the pattern of neo^{r} and LacZ expression was also as expected, being present only in embryos carrying a targeted locus. Analysis of the region encoding the trans-activation domain (downstream of the LacZ and neo^{r} cassettes), however, revealed that this portion of the gene was transcribed from both wild-type and targeted loci. In addition, a portion of the coding region 5′ to the LacZ and neo^{r} cassettes, and common to both wild-type and targeted loci, appeared to be transcribed only from the targeted locus. It is known that this region is transcribed under normal circumstances, and that mRNA secondary structure does not interfere with reverse transcription in this region, since this sequence is present in cDNA clones derived by reverse transcription Dunn, T. L. et al, 1995. We are therefore unable to explain the lack of expression of the 5′ region of Sox18 observed in these experiments.

Sox18^{-/-} Mice are Viable and Fertile.

Germline transmission of the targeted Sox18 allele was achieved by breeding male chimaeras derived from the two independently targeted ES cell lines. Genotyping of litters from intercrosses of F₁ Sox18^{+/-} heterozygotes revealed Sox18^{-/-} mice that appeared in Mendelian ratios. Breeding studies also indicated that the Sox18^{-/-} mice could interbreed, proving they were fertile (data not shown).

Anatomical Analysis of Sox18^{-/-} Embryos.

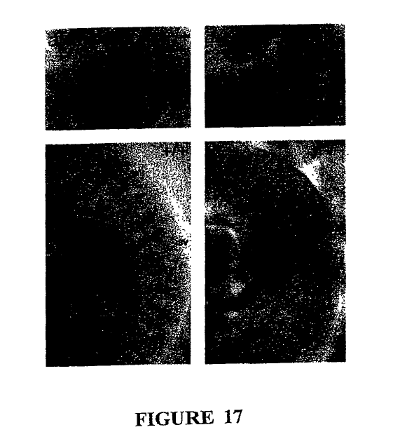

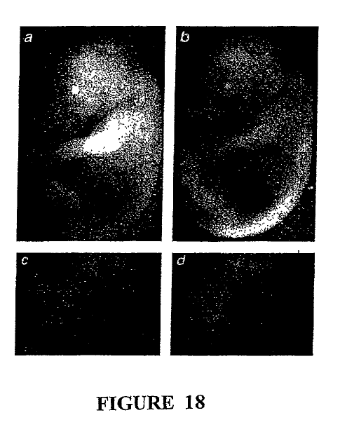

As Sox18^{-/-} mice appeared to have no reduced viability or fertility, there were likely to be no major cardiovascular defects associated with the targeted mutation. Immunohistochemistry using an antibody to the vascular endothelium marker, PECAM-1 (CD31), revealed that there were no major defects in the gross morphology of the heart of Sox18^{-/-} embryos (FIG. 17). In addition, the dorsal aortae, intersomitic vessels and vessels of the limb bud mesenchyme appeared grossly normal (FIG. 17). In addition there did not appear to be any haemorrhage or oedema in these embryos at 9.5 dpc. Likewise, at 14.5 dpc, no gross abnormalities nor haemorrhage or oedema were detected in Sox18^{-/-} embryos (FIG. 18). Furthermore, vibrissae follicles in Sox18^{-/-} embryos had developed in comparable numbers to that of littermates at the appropriate stage (FIG. 18).

Growth Rate of Sox18^{-/-} Mice.

As a general index of the wellbeing of Sox18^{-/-} mice, and to try to detect any subtle difference in the physiology of these mice, their growth rate relative to littermate controls was measured in terms of total weight. This analysis was performed using male littermates from both targeted lines. Over a two month period the mice were analysed, no statistically significant difference was observed between Sox18^{-/-} mice and Sox18^{+/-} and Sox18^{+/+} littermate controls at any stage (data not shown).

Coat Colour of Sox18^{-/-} Mice.

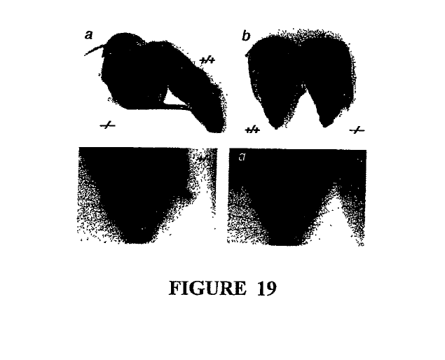

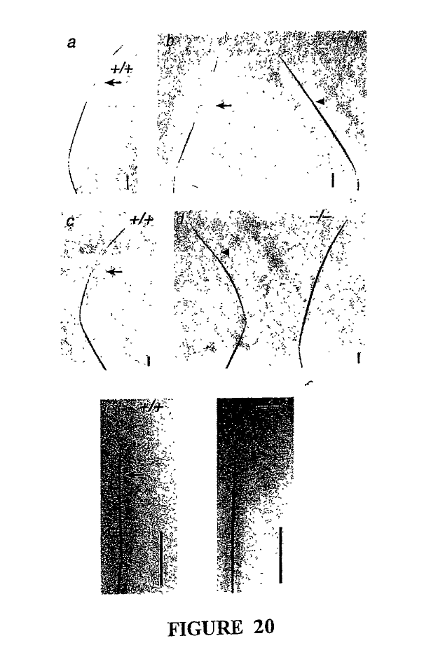

Segregation of alleles of coat colour genes amongst the stock of Sox18^{-/-} mice was observed due to the mixed genetic background of CD1 and 129/Sv. This facilitated the analysis of coat formation, with numerous pigmentation phenotypes, including albino, chinchilla and agouti, appearing in the stock of Sox18^{-/-} mice. Sox18^{-/-} mice had a slightly darker appearance than littermate controls on an agouti (FIG. 19) and chinchilla background (data not shown). Otherwise, no visible difference was detected in the coats of Sox18^{-/-} mice. Microscopic examination of the various hair types was conducted to further analyse the basis of the different coat pigmentation of Sox18^{-/-} mice. Again, this was done using mice with an agouti phenotype to facilitate examination of the sub-apical pheomelanin band typical of many agouti hairs. Examples of the four main types of pelage hairs, the guard hairs, awls auchenes and zigzags, appeared morphologically normal, though there was a difference in the pigmentation of many hair types between Sox18^{-/-} mice and littermate controls (FIG. 20). Guard hairs, which never have a sub-apical, pheomelanin band, appeared the same between Sox18^{-/-} mice and controls under microscopic examination (FIG. 20). Some awls from wild-type mice showed a sub-apical pheomelanin band, though most, like all those from Sox18^{-/-} mice, did not. Many auchenes and zigzags from Sox18^{-/-} mice showed either a reduced or completely absent pheomelanin band compared to controls.

Hair Formation in Sox18^{-/-} Mice.

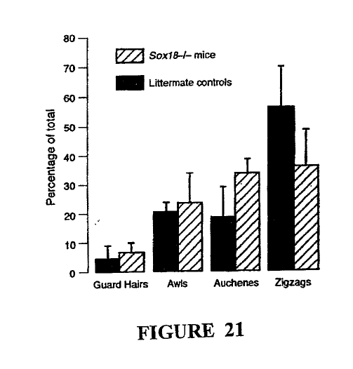

As some hair types are malformed and under-represented in Ra mice, namely the later forming auchenes and zigzag hairs, we conducted a survey of the proportion of pelage hair types amongst Sox18^{-/-} mice. From FIG. 21 it can be inferred that the proportion of guard hairs and awls was not significantly different between Sox18^{-/-} and littermate controls. There was a marked difference, however, in the proportion of zigzag hairs, which represented 56% of total hairs in wild-type mice to 36% in Sox18^{-/-} mice. It is worth noting that vibrissae hairs and follicles seemed normal at all stages in Sox18^{-/-} embryos and mice (FIGS. 18 and 19).

### Example 12

D Cloning and Functional Analysis of the Sox18 Gene

The results presented below illustrate the initial characterisation of the gene sequence of Sox18. Approximately 3.5 kb of the mouse gene locus has been sequenced and with this close scrutiny the inventors have elucidated a number of interesting facts. Most intriguingly, the inclusion of an intron within the encoded DNA binding domain, the exact position of which is reflected in other Sox gene sequences both within the same sub-group (specifically, Sox17 and Sox7 in group F) as well as a different one (Sox5 in group D). A detailed analysis via RT-PCR of the expression patterns of Sox18 is also described in the adult mouse, which shows predominant expression of Sox18 in the lung with moderate levels of expression observed in skeletal muscle, heart, intestine, spleen and kidney.

Materials and Methods

Cloning and automated cycle sequencing of the Sox 18 gene

The 855 bp PstI/XbaI fragment of Sox18 used to screen the murine genomic library (Lambda DashII vector, a gift from Philippe Soriano), has been reported before (Dunn etal., 1995). The radioactively labelled probe was generated using a Rediprime II Labelling Kit (Amersham) and the library was screened using standard procedures (Sambrook and Maniatis, 1989).

Sequencing of approximately 1620 bp of the 5′ flanking sequence was carried out via primer walking and subcloning. Briefly, the two primers chosen to complete the primer walking were, GMUQ450: 5′-GCTCCCTTTTTTCTTCCC-3′ [SEQ ID NO: 77] GMUQ480: 5′-GGAAAAGGAATGGGATTTGG-3′ [SEQ ID NO: 78]. Sub-clones were made using the restriction enzymes HaeIII, BglI and PvuII (Boehringer Mannheim). These restriction fragments were size fractionated on an agarose gel and purified with a Bandpure™ kit (Progen). The purified fragments were Klenow end-filled and ligated to pBS HincIII, these were then sequenced using M13 forward and reverse primers.

Sequencing of the pBS clones was carried out using double stranded DNA and automated sequencing performed with AmpliTaq™ DNA polymerase, FS, using ABI prism, BigDye terminator cycle sequencing. The ORF of Sox18 was sequenced completely on both strands using internal primers.

PCR-generated Random Mutagenesis

The technique used for mutagenic PCR was essentially as reported by (Rice et al, 1992), with the minor modifications as described by (Uppaluri and Towle, 1995). Briefly, in 50 mL, reaction mixtures contained 1 ng of template DNA to be mutagenised, 16 mM (NH4)2SO4, 67 mM Tris-HCl (pH8.8), 5 mM MgCl2, 0.5 mM MnCl2, 6.7 mM EDTA, 8.5 mg of Bovine Serum Albumin, 10 mM 2-Mercaptoethanol, 1 mM each dGTP, dTTP and dCTP, 400 mM dATP and 2.5U of Taq DNA polymerase (Pharmacia Biotech). Primers used at a final concentration of 1 mM, were GMUQ238: 5′-GCGAATTCCTGGAGCCGGGCCTCTTGCT-3′ [SEQ ID NO: 79] and GM-UQ239: 5′-GCGAATTCAGCAGAAGCTCGGGTCCCGTGC-3′ [SEQ ID NO: 80]. Cycling was carried out as follows: denaturation at 94° C. for 1 min, annealing at 55° C. for 1 min and extension at 72° C. for 2 min, for 25 cycles. A small aliquot (2 mL) of each mutagenic round was used to seed a further round of PCR mutagenesis with all new reaction components. Five or seven rounds were completed before the mutagenic PCR product was digested with EcoRI, gel purified and ligated to pBS cut with EcoRI. Single-stranded DNA was made for each clone (in excess of 200 clones) using standard protocols (Sambrook and Maniatis, 1989) and was sequenced as above.

RT-PCR for Sox18 Expression profile

RNA was prepared as for Northern Blotting, quantitated using the Ultraspec™ 2000, spectrophotometer (Pharmacia Biotech) and then quantitated by eye against a known quantity of RNA on a 1.2% agarose/1% formaldehyde gel. 5 mg of RNA from each tissue was then subjected to RT-PCR as outlined in the Superscript II manufacturers protocol (Life Technologies). Briefly, the RNA was resuspended with 250 ng of random primer in a total of 12 mL of DEPC treated water. This was then heated at 70° C. for 10 min and chilled on ice. First strand buffer was then added along with 10 mM DTT and a 0.5 mM dNTP mix, incubated at 25° C. for 10 min and then transferred immediately to 42° C. for 2 min before the addition of Superscript II enzyme. The incubation at 42° C. was extended for a further 50 min and the reaction inactivated at 70° C. for 15 min.

A maximum of 2 mL of this reaction volume was then used in PCR with either primer pair GMUQ238 and 239 (see above) or GMUQ529: 5′-GCGCGGCCTCCCTGTCACCAACG-3′ [SEQ ID NO: 81] and 530: 5′-CCAAAGGCGGTGGGAAGAAGGAG-3′ [SEQ ID NO: 82]. At all times a reaction control was used to check for PCR contamination as well as a control for misleading results from potential genomic DNA contamination of the RNA samples.

To test for the presence of RNA transcripts containing the intron, we used primers GMUQ401: 5′-GCGAATTCAC-CATGCAGAGATCGCCGCCCGGCTACG-3′ [SEQ ID NO: 83] and GMUQ503: 5′-GCGGAATTCCTGTAGGCGAAGG-GAGCCTG-3′ [SEQ ID NO: 84]. These primers only amplify cDNA that contains the intron. A misleading factor for this experiment is that the RNA from which the cDNA was made may contain contaminating genomic DNA, which would amplify as well. To overcome this difficulty we amplified the intron containing fragment in parallel, from equivalent amounts of cDNA and the respective RNA (a no RT control). If there was more product in the RT sample than the RNA control then in that tissue at least some of the mRNA was not spliced and would still contain the intron.

Results

Cloning and Sequencing of the Sox18 Gene

We screened a murine λ genomic library for Sox18. Four overlapping clones were obtained and a 3.3 kb XbaI fragment containing the entire open reading frame of 1592 bp as well as approximately 1750 bp of flanking 5′ and 3′ sequences was cloned into pBluescript II. This clone and subclones of it were used for all genomic sequences reported here.

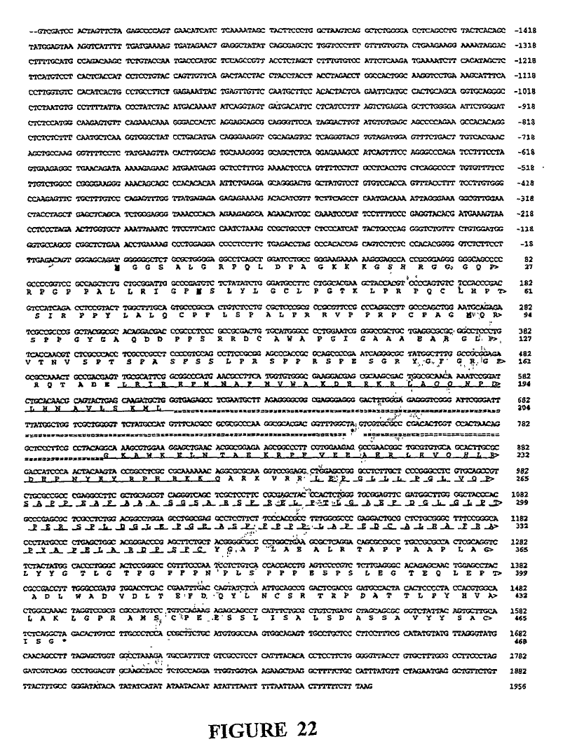

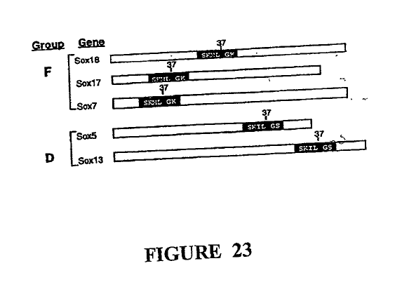

The DNA and amino acid sequence of Sox18 is depicted in FIG. 22. This sequence revealed a single intron located in the HMG box. This occurs at the precise amino acid position reported for Sox genes, 17, 5, 6 and 13 (FIG. 23). Furthermore, the C. elegans group D open reading frame W01C8.2 (Wegner, 1999) contains an intron at the same position as the mammalian genes, arguing for a conservation of intron positions across species and even throughout evolution. A partial Sox18 cDNA sequence has been described previously (Dunn, T L, et al., 1995; W097/04090). The present analysis reveals an additional in-frame 91 amino acids containing a further two methionine residues. Thus, for the first time, the entire ORF for murine Sox18 is presented, which encodes a protein of 468 amino acids.

The putative initiator methionine is in a relatively favourable context, as elucidated by Kozak (for review see Kozak, 1996). Of the two preferred (underscored) nucleotides at 23 and 14, the guanine at 14 is conserved, (nt 26)..GAGCA-GaugG.(nt 14). Furthermore, two other methionines which are encoded downstream of the first methionine have relatively weaker contexts, with no preferred nucleotides conserved for the methionine at nt 1117. Interestingly, the methionine at nt 1273 has a purine in the correct context (at nt 1270) although it is a guanine and not the preferred alanine. In addition there are multiple stop codons present in all three frames in the 5′ UTR. It is interesting to note that there are two small ORFs encoded upstream of the main initiator (at nt positions 2588 to 2472 and 2228 to 2100). Such ORFs may pose a problem for translation initiation at the initiator codon, only if they are encoded with a favourable context and do not lead to a terminator codon. In this case, they both have very weak contexts, with neither of the preferred nucleotides being present and both are only short ORFs terminating before translation initiation.

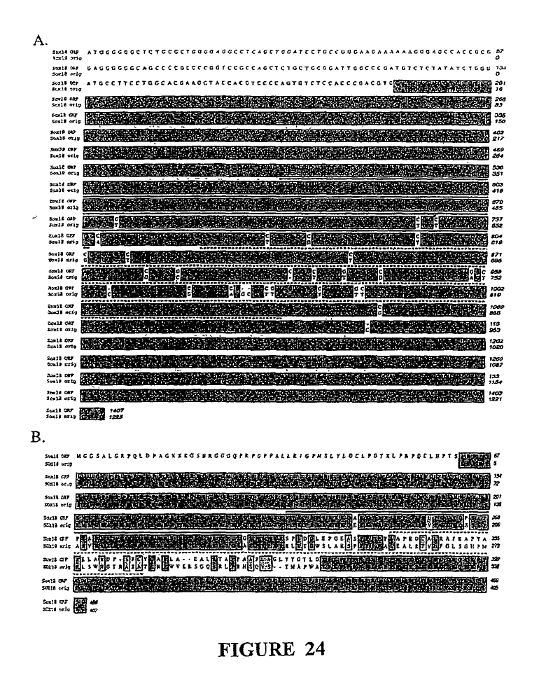

This situation is not unusual for important regulatory genes and it is known that genes encoding growth factors, receptors, transcription factors and the like may actually utilise factors such as upstream ORFs and less than efficient initiators to limit the effectiveness of translation as a way of controlling their expression (for review see Kozak, 1991).The presence of these elements in the gene sequence strongly suggest that here we describe for the first time the complete open reading frame for murine Sox18.The Sox18 gene encodes a protein of 468 amino acids. FIG. 24A, B present a comparison of the new, denoted ‘Sox18 ORF’ and previously published (Dunn et al., 1995), denoted ‘Sox18 orig’ nucleotide and amino acid sequences, respectively.

Haf-2 is the Human Orthologue of Sox18

In 1996, the isolation and characterisation of two human cDNAs that were shown to bind the HE2 region of the human heavy chain (IgH) enhancer was reported (Stevens et al., 1996). These cDNAs were designated HAF-1 and -2(HMG-box activating factors) and were found to be expressed in both B lymphocytes and other cells (HeLa). Both proteins were found to be transcriptional activators in a Ga14 hybrid assay and could regulate transcription via bind-ing to a minimal promoter derived from the IgH enhancer. HAF-1 was reported to be the human homologue of mouseSox17 with an amino acid similarity in the HMG region of 92%, whereas HAF-2 similarly had a reported identity of 96% with the Sox8 HMG domain.

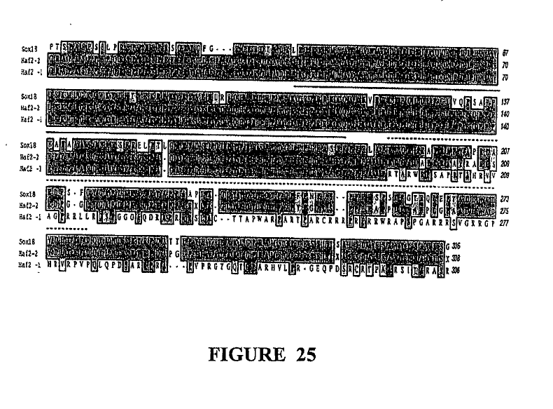

A comparison of the new Sox18 and the HAF-2 DNA and protein sequence (denoted Haf-2-1 in FIG. 25), particularly within the HMG box, left little doubt that the two clones were murine/human orthologues. There was remarkable DNA and amino acid sequence similarity, differing by only three amino acids out of 80, but outside of this region the identity broke down almost completely. However, when the nucleotide and amino acid sequences of both Sox18 andHAF-2 were compared directly by alignment with Macvector using the ClustalW algorithm (Thompson et al., 1994), it was found that with the addition of a number of frame shifts in the HAF-2 sequence, the amino acid sequence (denotedHaf-2-2) became remarkably similar to Sox18. The resulting deduced protein shows a similarity with Sox18 of 84% as compared to 57% for the original.3.3. Analysis of the Sox18 activation domain by random mutagenesis: the amino acids between 313 and 345 are critical to the integrity of the Sox18 activation domain Sox18 is capable of trans-activating gene expression in an AACAAAG-dependent manner. Furthermore, GAL4 hybrid analysis has demonstrated that Sox18 is an efficient trans-activator of gene expression (Hosking et al., 1995). We localised the activation domain to 92 amino acids between amino acids 253 and 345 in Sox18. This region was denoted as al. 60-255 by Hosking et al. (1995) Hosking et al., (1995). Further, we demonstrated that 8-Bromo cAMP and okadaic acid, which stimulate PKA and inhibit serine/threonine protein phosphatases, respectively, both increased the trans-activity of Sox18. Thus the phosphorylation status of Sox18 may play a key regulatory role in the activity of this protein. A computer scan (Subsequence Search, MacVector) of the 92 aa activation domain suggests two potential protein kinase C (PKC) phosphorylation sites, at serine residues 278 and 281. A potential casein kinase II (CKII) phosphorylation site was indicated at serine 281.

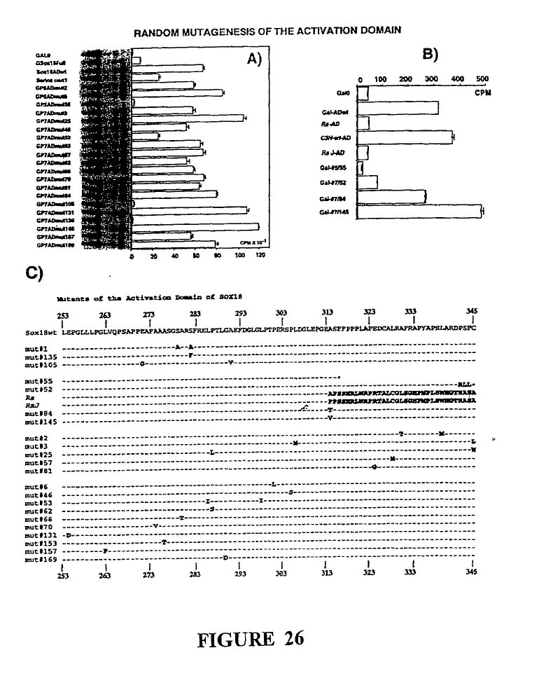

A rigorous random mutagenesis of the activation domain has been performed to identify the critical residues involved in transcriptional activation (FIG. 26). The mutated activation domains were subcloned downstream of the GAL4 DBD, and examined for their ability to trans-activate the GAL4 dependent reporter, G5E1bCAT (FIG. 26C). Mutagenesis of the putative twin PKC and CKII site SARS to AARA (Serinemut1) or to SARF (mut #135) resulted in reduced activity of the trans-activation domain, implicating these sites in Sox18 function (FIG. 26; mut#1 and #135, 3 and 20-fold reduction, respectively). The significance of these overlapping, potential PKC and CKII sites, in spatiotemporal expression, has yet to be determined.

Furthermore, mutants, #52 and #55, demonstrated that the carboxy terminus of the Sox18 activation domain was necessary for efficient trans-activation and complementation of the GAL4 DBD (FIG. 26). Mutant #55 introduced a stop codon after amino acid residue 314, that suggested residues between amino acids 315 and 346 were required for the function of the Sox18 activation domain. Mutant #52 that introduced changes between amino acids 342 and 345 also compromised effective trans-activation. Curiously, this mutation occurs at a potential cAMP protein kinase site changing the amino acid sequence from ARDS to ARDR. It has been described above that Sox18 is mutated in the classical mouse mutant ragged (Ra) (Pennisi et al., 2000). Interestingly, the defects in Ra and the allelic mutant RaJ create frameshifts in the trans-activation domain from amino acids 314 and 313, respectively, that impairs its function (see FIG. 26B). The ragged transcriptional defects are consistent with the data from Mut#55 that introduced a stop codon at position 315. These data reinforced that the region between amino acid residues 315 and 346 was necessary for effective trans-activation.

This observation was further validated by assaying the activity of the Sox18 activation domain from the Ra and RaJ mice in the GAL4 hybrid system, relative to the mutants, 55 and 52. As additional controls we assayed the point mutants #84 and #145, i.e. A313T and A313V (that changed the identical residue as the mutation in the Ra mice without the subsequent frameshift). This experimental analysis verified that the Ra, #52 and #55 mutants compromised the function of the activation domain. However, mutants #84 and #145, had negligible effects on trans-activation by this region, consistent with the notion that the frameshift produced the effect rather than the mutation of the specific residue (FIG. 26B). Moreover, in support of this hypothesis, the site specific mutants, #2, 3, 25, 57 and 81 in the C-terminal region of the activation domain, functioned efficiently in the GAL4 hybrid system suggesting that the integrity of the activation domain was not compromised by point mutations in this region.

The other point mutants 6, 46, 53, 62, 66, 70, 131, 153,157, and 169 had negligible effects on the effective function of the activation domain. In summary the analysis indicated that the SARS potential phosphorylation site and the amino acid residues between position 313 and 345 were critical to Sox18 function.

Expression of Sox18 in the Adult Mouse

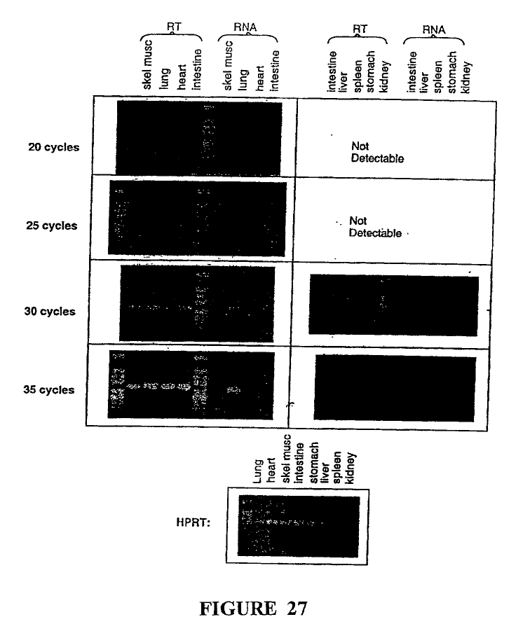

It has been demonstrated via Northern analysis, that Sox18 mRNA is predominantly expressed in lung tissue (Dunn et al., 1995). Weak expression was detected in skeletal muscle, heart and intestine. To complement and extend the study of the expression profiles of Sox18 in the adult mouse, the distribution of Sox18 mRNA expression was examined by RT-PCR (see FIG. 27). RT-PCR analysis demonstrated that highest expression in the adult mouse occurs in the lung tissue with moderate levels in heart and skeletal muscle, intestine, spleen and kidney. A low level of expression was also evidenced in the stomach, with no detectable amount in the adult liver.

In addition, we further employed RT-PCR to elucidate whether in any of the tissues studied, the intron was expressed in the mature transcript. If this was the case then the predicted protein would contain sufficient amino acid residues of the HMG box to bind to DNA sequence-specifically (data not shown), but would not be able to trans-activate expression of downstream genes. Thus this potential protein may be used as a dominant negative regulator for Sox18 regulated gene expression. We have determined that the intron does not appear in the mature transcript for any of the adult mouse tissues studied (data not shown).

### Example 13

E Structure, Mapping and Expression of Human Sox18

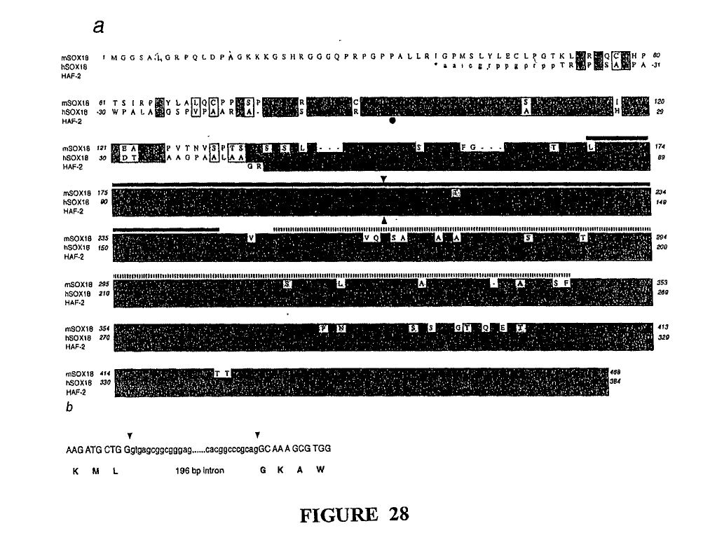

The inventors sought to characterise human SO18. Searches of the GenBank database revealed the existence of a human expressed sequence tag (EST) clone (AI744846, Genome Systems Inc, Berkeley, Miss., USA) with significant homology to murine Sox18. Both strands of this 1730 bp EST clone was sequenced using the ABI Prism BigDye Terminator system (ABI, Foster City, Calif., USA). Cycling was conducted in a DNA thermal cycler (Perkin Elmer Cetus) thus: 95° C. for 5 minutes, twenty five cycles of 96° C. for 30 s, 50° C. for 15 s and 60° C. for 4 min, with the following primers: primer A, 5′-GCCCAGAGGAGAGCAGC-3′ [SEQ ID NO: 85]; primer B, 5′-GCGCCGCAAGAA GCAGG-3′ [SEQ ID NO: 86]; primer C, 5′-GCTGCTCTCCTCTGGGC-3′ [SEQ ID NO: 87]; primer D, 5′-CAGGAGGCCGGGCTCCA-3′; [SEQ ID NO: 88] primer E, 5′-GCCCGCCGCCGATCTGT-3′ [SEQ ID NO: 89]; primer F, 5′-GCCCGTTCCACC TCCCA-3′ [SEQ ID NO: 90]; primer G, 5′-CCCTACGCGCCCACCGA-3′ [SEQ ID NO: 91]; primer H, 5′-TCGGTGGGCGCGTAGGG-3′ [SEQ ID NO: 92]; M13 Forward, 5′-GTAAAACGACGGCCAGT-3′ [SEQ ID NO: 93] and M13 Reverse, 5′-CAGGAAACAG CTATGAC-3′ [SEQ ID NO: 94]. Sequences were analysed with Sequencher 3.0 software (Gene Codes Corporation, Ann Arbor, Mich., USA). This sequence has been assigned GenBank accession number AF270652. In view of the high level of sequence identity with mouse Sox18 (FIG. 28a), it was concluded that the human sequence represents human Sox18.

The human sequence is shorter at the 5′ end by 150 bp (50 amino acids) than the extended mouse Sox18 coding sequence we have recently determined (Hosking et al., manuscript submitted; GenBank accession number AF288518; FIG. 28a). However, intensive efforts involving library screening and 5′-RACE PCR have failed to provide further 5′ human cDNA sequence. Searching high-throughput human genome sequence databases (http://www.ncbi.nlm.nih.gov/BLAST/) revealed two clones from human Chromosome (Chr) 20 homologous to human Sox18. One of these (Sanger Centre clone RP11-238J15; GenBank AL356790) overlaps with the 5′ end of the EST clone, and provides an additional 37 bp of 5′ sequence bounded by an in-frame termination codon TGA (FIG. 28a). Homology between the human and mouse sequences breaks down shortly 5′ to the first methionine codon in the human sequence (FIG. 28a). These observations suggest that the cDNA sequence presented here contains the entire coding sequence of human SOX18, and that the mouse and human proteins differ at their N-termini.

It has previously been reported that HMG box-activating factor 2 (HAF-2) is expressed in both B cells and non-B cells, and that the protein product encoded by this gene binds the human Ig heavy chain enhancer and is a potent activator of transcription (Stevens et al. 1996). HAF-2 shows extremely high homology to Sox18 at the DNA level, though the reported amino acid sequence varied considerably from that of Sox18 outside the HMG box. This partial HAF-2 clone was resequenced and the corrected amino acid sequence was found to be identical to human SOX18 apart from two amino acids at the N-terminus (FIG. 28a). It therefore appears that HAF-2 is human Sox18.

Comparison of cDNA and genomic sequence (Sanger Centre clone RP13-152015; AL355803) indicates the presence of a single intron of 196 bp (FIG. 28b). The position of the intron in human SOX18 is similar to intron sites in mouse Sox18, -5, -7, -13 and -17 (Kanai et al. 1996; Wunderle et al. 1996; Roose et al. 1999; Taniguchi et al. 1999, suggesting that this subset of SOX genes shares a common ancestry.

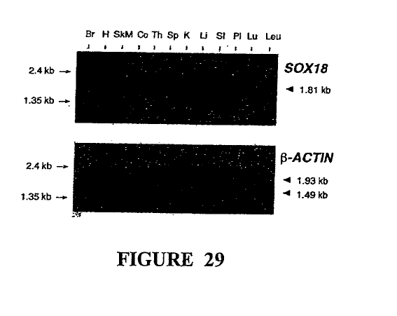

Sox18 expression was investigated by hybridisation of a human multi-tissue northern filter (Clontech, Palo Alto, Calif., USA). Strong expression was observed in the heart, with signals also detected in brain, skeletal muscle, spleen, kidney, liver, small intestine, placenta and lung (FIG. 29). Weaker signals were also detected in colon and thymus. In all tissues where expression was seen, a single transcript of 1.81 kb was observed, indicating that SOX18 is not alternatively spliced. It is perhaps surprising that no Sox18 expression was detected in peripheral blood leucocytes in this study given that Stevens et al. (1996) reported expression and cloning of HAF-2 from B-cells. This may, however, represent low levels of expression not detectable by northern hybridisation. The widespread pattern of Sox18 expression in human tissues may represent blood vessel remodelling that is occurring in these tissues, given the strong expression of Sox18 in developing blood vessels as described herein, or may represent another function for SOX18.

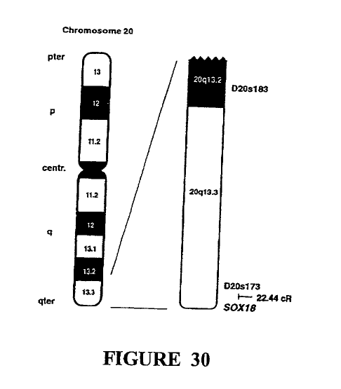

The chromosomal localisation of Sox18 was also determined using the GeneBridge™ 4 human-hamster radiation hybrid panel (Human Genome Mapping Project, UK). PCR was performed for the detection of human SOX18 amongst the panel DNA samples using human Sox18-specific primers (primer 1, 5′-CCGACGTGGACCTCACCG AGTTCG-3′ [SEQ ID NO: 95]; primer 2 5′-AGGTGGCCAGAAGCCCAGGAAGGG-3′ [SEQ ID NO: 96]). Cycling was conducted in a DNA Thermal Cycler (Perkin Elmer Cetus) thus: 95° C. for 5 min; thirty cycles of 95° C. for 30 s, 67° C. for 30 s and 72° C. for 1 min; 72° C. for 10 min. PCR products were separated by electrophoresis on 1.5% agarose gels and visualised with ethidium bromide under ultra-violet light. Visible bands were scored as positive for Sox18. Resulting score data from the panel were analysed by software accessed through the world-wide web at http://www@genome.wi.mit.edu. Sox18 was localised to the long arm of Chr 20, 22.44 cR distal from the anonymous marker D20s173 (LOD score greater than 13). This places SOX18 close to the telomere at 20q13.3 (FIG. 30). This is a region of conserved synteny with distal mouse Chr 2, where mouse Sox18 has previously been mapped (Greenfield et al. 1996). Orthologues of the genes Acra4 and Gnas, which flank Sox18 in the mouse genome, have also been mapped to human 20q13.3 (Levine et al. 1991; Anand and Lindstrom 1992; Juppner et al. 1998), further confirming this map location for SOX18.

The disclosure of the priority Austaralian patent application No. PQ6457 filed on Mar. 24, 2000 is incorporated herein by reference in its entirety. The disclosure of every patent, patent application, and publication cited herein is also incorporated herein by reference in its entirety. The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

Abbott, C. etal., 1994, Genomics, 20: 94-98

Abremski, K., and R. Hoess. 1984. Journal of Biological Chemistry, 259:1509-1514.

Adams et al., 1993, Cancer Res. 53: 4026-4034

Adelman et al. 1983, DNA 2:183

Algin et al 1994, Tetrahedron Letters 35: 9633-9636

Altschul et al., 1997, Nucl. Acids Res. 25:3389

Anand, R., Lindstrom, J. 1992, Genomics, 13: 962-967.

Andrews, et al. 1990, Munksgaard, Copenhagen, 28: 145-165.

Appel et al. 1992, Immunomethods 1: 17-23

Ausubel et al., John Wiley & Sons, Inc. 1994-1998

Balkenhohl et al. 1996, Angew. Chem. Int. Ed. Engl. 35: 2288-2337

Barany et al. 1979, In The Peptides, eds. E. Gross and J. Meienhofer (Academic Press: N.Y.), 2: 3-254

Barr et al., 1991, Science 254: 1507-1512

Bell, D M, et al., 1997, Nature Genetics 16: 174-178

Bertling et al., 1991, Biotech. Appl. Biochem. 13: 390-405

Bi, W., Deng, J. M., Zhang, Z., Behringer, R. R. & de Crombrugghe, B., 1999, Nature Genet. 22, 85-89.

Boehm, T., Folkman, J., Browder, T., and O'Reilly, M. S., Nature, 390: 404-407

Bowles, J. and Koopman, P., 1996, Gene Families: Structure, Function, Genetics and Evolution (R S Holmes and H A Lim, Eds.) World Scientific Publishing Co., Singapore. pp. 177-191.

Bullock et al., 1987, BioTechniques, 5: 376-379

Calabretta et al., 1993, Cancer Treat. Rev. 19: 169-179

Carmeliet, P. et al., 1996, Nature, 380: 435-439.

Carter et al. 1986, Nucl. Acids. Res., 13: 4331

Carter, T. C. & Phillips, R. J. S. Ragged, 1954, J. Hered. 45: 151-154.

Cattanach, B. M., Kirk, M., 1985, Nature, 315: 496-498.

Chang, M W et al., 1995, Science 2671: 518-622

Chen et al, 1987, Mole. Cell Biochem. 7: 2745-2752

Chen, S., Dowhan, D., Hosking, B., and Muscat, G., 2000, Genes Dev., 14: 1209-1228

Christiansen, J. H., et al., 1995, Mech. Dev., 51: 341-350

Coligan et al., 1991, Current Protocols in Immunology (John Wiley & Sons, Inc.)

Coligan et al., Current Protocols in Protein Science (John Wiley & Sons, Inc. 1995-1997, in particular Chapters 1, 5 and 6.

Craik, C. S., 1985, Science 228: 291-297

Creighton, The Proteins, W. H. Freeman & Co., N.Y.; Chothia, 1976, J. Mol. Biol., 150: 1

Cronin, et al., 1988, Biochem. 27: 4572-4579

Cumber et al., 1992, J. Immunol. 149: 120-126

Davis et al., Microbiology (Harper and Row: New York, 1980), pages 237, 245-247, and 274

Davies & Riechmann, 1994, FEBS Lett. 339: 285-290

Deveraux et al. 1984, Nucleic Acids Research 12, 387-395

Dhawan et al., 1991, Science 254 1509-1512

Dooley and Houghten, 1993, Proc. Nat. Acad. Sci. U.S.A. 90: 10811-10815

Dry, F. W. 1926, Journal of Genetics, 16:287-340.

Dumont, D. J. et al., 1994, Genes Dev., 8: 1897-1909

Dunn T L, Mynett-Johnson L, Wright E M, Hosking B M, Koopman P A, and Muscat G E O, 1995, Gene, 161:223-225

Eichler and Houghten, 1993, Biochemistry 32: 11035-11041

Eichler et al. 1995, Medicinal Research Reviews 15(6): 481-496

Fodor et al., (1991, Science 251:767-777

Fong, G. H., Rossant, J., Gertsenstein, M. & Breitman, M. L., 1995, Nature 376, 66-70.

Foster, J. W., M. A. Dominguez-Steglich, S. Guioli, C. Kwok, P. A. Weller, J. Weissenbach, S. Mansour, I. D. Young, P. N. Goodfellow, J. D. Brook, and A. J. Schafer. 1994, Nature, 372:525-530.

Furka et al. 1988, 14th Int. Congr. Biochem., Prague, Czechoslovakia 5: 47; 1991, Int. J. Pept. Protein Res. 37: 487-493

Gasparini, G., Bonoldi, E., Viale, G. et al, 1996, Int. J. Cancer., 21(69): 205

Gerit, J. A. 1987, Chem. Rev. 87: 1079-1105

Geysen et al. 1986, Mol. Immunol. 23: 709-715

Geysen 1986, Immun. Today 6: 364-369

Glockscuther et al. Biochem. 29: 1363-1367

Green, E. L. & Mann, S. J.,. J. Hered. 52, 223-227 (1961).

Greenfield, A., Dunn, T., Muscat, G. & Koopman, P,. Genomics 36, 558-559 (1996).

Gubbay, J., Collignon, J., Koopman, P., Capel, B., Economou, A., Munster-berg, A., Vivian, N., Goodfellow, P., Lovell-Badge, R., 1990, Nature, 346: 245-250.

Hahn, H. et al., 1996, J. Biol. Chem., 271: 12125-12128.

Hamers-Casterman et al. 1993, Nature. 363: 446-448

Herbertson, B. M. & Wallace, M. E., 1964, J. Med. Genet., 1: 10-23.

Hogan, B., R. Beddington, F. Costantini, and E. Lacy. 1994. Manipulating the Mouse Embryo: A Laboratory Manual, 2nd ed. Cold Spring Harbour Laboratory Press, Cold Spring Harbour, New York.

Hol,. et al., 1989, Roberts, S. M. (ed.); Royal Society of Chemistry; pp. 84-93.

Hol, W. G. J. 1986, Agnew Chem. Int. Ed. Engl. 25: 767-778

Hol, W. G. J. 1989, Arzneim-Forsch. 39: 1016-1018

Hollingsworth, H., Kohn, E., Steinberg, S. M. et al, 1995, Am J. Pathol., 147: 33

Hosking, B. M., Muscat, G. E. O., Koopman, P. A., Dowhan, D. H. & Dunn, T. L., 1995, Nucleic Acids Res. 23: 2626-2628.

Houghten et al. 1991, Nature 354: 84-86; 1992, BioTechniques 13: 412-421

Hruby et al 1994, Reactive Polymers 22: 231-241

Iseki, S. et al., 1996, Biochem. Biophys. Res. Commun. 218: 688-693

Joyner, A. L. 1993. Gene targeting: a practical approach. Oxford University Press, Oxford, UK.

Jüppner, H., Schipani, E., Bastepe, M., Cole, D. E., Lawson, M. L., Mannstadt, M., Hendy, G. N., Plotkin, H., Koshiyama, H., Koh, T., Crawford, J. D., Olsen, B. R., Vikkula, M., 1998, Proc. Natl. Acad. Sci. USA, 95: 11798-11803.

Kanai, Y., Kanai-Azuma, M., Noce, T., Saido, T. C., Shiroishi, T., Hayashi, Y., Yazaki, K., 1996, J. Cell Biol., 133: 667-681.

Kates et al 1993, Tetrahedron Letters 34: 1549-1552

Kazal et al, 1996, Nature Medicine 2:753-759

Kitajima, I., Shinohara, T., Bilakovics, J., Brown, D. A., Xu, X., and Nerenberg, M., 1992, Science, 258: 1792-1795

Knowles, J. R., 1987, Science 236: 1252-1258

Köhler and Milstein 1975, Nature 256, 495-497

Koopman, P. et al., 1990, Nature, 348: 450-452

Korn, R., M. Schoor, H. Neuhaus, U. Henseling, R. Soininen, J. Zachgo, and A. Gossler., 1992, Mechanisms of Development, 39: 95-109.

Kostelny et al., 1992, J. Immunol. 148: 1547-1553

Kozak, M., 1991, J. Cell Biol, 115: 887-903.

Kozak, M., 1996, Mamm. Genome, 7: 563-574.

Krebber et al. 1997, J. Immunol. Methods; 201(1): 35-55

Ku & Schultz, 1995, Proc. Natl. Acad. Sci. USA, 92: 652-6556

Kuhlbrodt, K., Herbarth, B., Sock, E., Hermans-Borgmeyer, I., and Wegner, M., 1998, J. Neurosci, 18: 237-250

Lam et al. 1991, Nature 354: 82-84

Langer, R. and Vacanti, J. 1993, Science 260: 920-926

Lardner et al. U.S. Pat. No. 5,223,409

Leatherbarrow, R. 1986, J. Prot. Eng. 1: 7-16

Leifer et al., 1993, Proc. Natl. Acad. Sci. USA 90(4): 1546-1550

Levine, M. A., Modi, W. S., O'Brien, S. J. 1991, Genomics, 11: 478-479.

Lin, Q. et al., 1998, Development, 125: 4565-4574

Liu et al., 1996, J. Am. Chem. Soc. 118:1587-1594

Lowman, et al. 1991, Biochem. 30: 10832-10838

Mann, S. J., 1963, Genet. Res. 4: 1-11.

Mansour, S. L., K. R. Thomas, and M. R. Capecchi., 1988. Nature, 336: 348-352.

Markland, et al. 1991, Gene 109: 13-19

Marlowe 1993, Biorganic & Medicinal Chemistry Letters 3: 437-44

McMurray et al 1994, Peptide Research 7: 195-206

McPherson, A. 1990, Eur. J. Biochem. 189: 1-24

Miller, 1992, Nature 357: 455-460

Mosman, 1983, J. Immunol. Meth. 65: 55-63

Muller, D W M et al. 1994, Circulation Research 75: 1039-1049

Mulligan, R. C., 1993 Science 260: 926-932

Munder et al. 1999, Appl. Microbiol. Biotechnol. 52(3): 311-20

Nabel, R. et al. 1990, Science 249: 1285-1287; and 1993, Nature 362: 844-846

Nabel, E. G. et al., 1993, Nature 362: 844-846

Nagy, A., J. Rossant, R. Nagy, W. Abramow-Newerly, and J. C. Roder, 1993, Proc. Natl. Acad. Sci. USA, 90: 8424-8428.

N g, L J, et al., 1997, Developmental Biology 183: 108-121

O'Danos et al. 1988, Proc. Natl. Acad. Sci. USA 85: 6460-6464

Ohno, T. et al. 1994, Science 265: 781-785

O'Reilly, M. S., Holmgren, L., Chen, C., and Folkman, J., 1996, Nature Medicine, 2: 689-692

Pack P. Plünckthun, 1992, Biochem. 31: 1579-1584

Pallin and Tam 1995, J. Chem. Soc. Chem. Comm. 2021-2022

Pennisi, D., Gardner, J., Chambers, D., Hosking, B., Peters, J., Muscat, G., Abbott, C., and Koopman, P., 2000, Nature Genet., 24: 434-437

Phizicky and Fields, 1994, Microbiol. Rev. 59(1): 94-123

Pingault, V. et al., 1998, Nature Genet. 18: 171-173

Pinilla et al. 1992, BioTechniques 13: 901-905; 1993, Gene 128: 71-76

Plückthun et al 1996, In Antibody engineering: A practical approach. 203-252

Pompili, V. J. et al., 1995, Arteriosclerosis Thrombosis and Vascular Biology 15: 2254-2264

Price et al 1987, Proc. Natl. Acad. Sci. USA 84: 156-159

Priestle, J., 1988, J. Mol. Graphics 21: 572

Puri, M. C. et al., 1995, J. Embo, 14: 5884-5891

Reiter et al. 1994¹, J. Biol. Chem. 269: 18327-18331

Reiter et al. 1994², Biochem. 33: 5451-5459

Reiter et al 1994³. Cancer Res. 54: 2714-2718

Refetoff, S., Weiss, R., and Usala, S., 1993, Endocr. Rev., 14: 284-335

Rice, G. C., Goeddel, D. V., Cachianes, G., Woronicz, J., Chen, E. Y., Williams, S. R.,

Leung, D. W., 1992, Proc. Natl. Acad. Sci. USA, 89: 5467-5471.

Robbins et al., 1998, Trends Biotechnol. 16: 35

Roberge et al 1995, Science 269: 202

Roberts, et al. 1992, Proc. Natl. Acad. Sci. (U.S.A.) 89: 2429-2433

Roelink, H. et al., 1995, Cell, 81: 445-455

Roose, J., Korver, W., de Boer, R., Kuipers, J., Hurenkamp, J., Clevers, H., 1999, Genomics, 57: 301-305.

Rose et al., 1991, BioTech. 10: 520-525

Salmons et al., 1993, Hum. Gen. Ther. 4: 129-141

Sambrook, J., Maniatis, T., 1989. Molecular Cloning: A Laboratory Manual, second ed., Cold Spring Harbour Press, Cold Spring Harbour, Laboratory Press, New York. in particular Sections 16 and 17

Schepers, G, et al., Nucleic Acids Research, in press. Accepted January 2000.

Schmidt and Langer 1997, J. Peptide Res. 49: 67-73

Sekiya, I, et al., 2000, J. Biol. Chem., in press. Accepted February 2000

Shalaby F, Rossant J, Yamaguchi T P, et al., 1995, Nature, 376: 62-66.

Shaw, W. V., 1987, Biochem. J. 246: 1-17

Shigekawa et al., 1988, BioTech. 6: 742-751

Slee, J., 1957, J. Genet. 55: 100-121.

Slee, J. 1957², J. Genet. 55, 570-584.

Smith, G. P. 1985, Science 228: 1315-1317

Smith, et al. 1990, Science 248: 1126-1128

Sooknanan et al., 1994, Biotechniques 17:1077-1080

St Jacques, B. et al., 1998, Curr. Biol. 8: 1058-1068

Stein, C. A. and Cheng, Y -C., 1993, Science, 261: 1004-1012

Stevens, S., Ordentlich, P., Sen, R., Kadesch, T., 1996, J. Immunol,. 157(8): 3491-3498.

Taniguchi, K., Hiraoka, Y., Ogawa, M., Sakai, Y., Kido, S., Aiso, S., 1999, Biochim Biophys Acta, 1445: 225-231.

Theiler, K. The House Mouse (Springer-Verlag, Berlin, 1972)

Thompson, J. D., Higgins, D. G., Gibson, T. J., Clustal, W., 1994, Nucleic Acids Res,. 22: 4673-4680.

Tumelty etal 1994,J. Chem. Soc. Chem. Comm. 1067-1068

Tyagi et al., 1996, Proc. Natl. Acad. Sci. USA 93: 5395-5400

Uppaluri, R., Towle, H. C., 1995, Mol. Cell Biol., 15: 1499-1512.

Viera et al. 1987, Methods Enzymol. 153:3

von der Leyen et al., 1995, Proc. Natl. Acad. Sci. USA 92: 1137-1141

Wagner T, Wirth J, Meyer J, Zabel B, Held M, Zimmer J, Pasantes J, Bricarelli FD, Keutel J, Hustert E, Wolf U, Tommerup N, Schempp W, and Scherer G., 1994, Cell, 79:1111-1120

Wallace, M. E., 1979, Heredity, 43: 9-18

Ward et al. 1989, Nature 341: 544-546

Watson, J. D. et al., “Molecular Biology of the Gene”, Fourth Edition, Benjamin/Cummmings, Menlo Park, Calif., 1987

Webber et al. 1995, Mol. Immunol. 32: 249-258

Wegner, M., 1999, Nucleic Acids Res. 27: 1409-1420

Wells et al. 1985, Gene, 34: 315

Wells et al., 1986, Philos. Trans. R. Soc. London SerA, 317: 415

Wheatley, S. C., C. M. Isacke, and P. H. Crossley., 1993, Development, 119: 295-306.

Wilks, et al., 1988, Science 242: 1541-1544

Winter and Milstein 1991, Nature 349:293

Wolff et al., 1990, Science 247: 1465-1468

Wood, S. A., N. D. Allen, J. Rossant, A. Auerbach, and A. Nagy, 1993, Nature, 365: 87-89.

Wright E, Hargrave M R, Christiansen J, Cooper L, Kun J, Evans T, Gangadharan U, Greenfield A, and Koopman P., 1995, Nature Genet, 9: 15-20

Wright E M, Snopek B, Koopman P., 1993, Nucleic Acids Research, 21: 744.

Wunderle, V. M.; Critcher, R.; Ashworth, A.; Goodfellow, P. N. 1996, Genomics 36: 354-358

Xie, W. F., et al., 1999, J. Bone Miner. Res., 14: 757-763

Yamaguchi, T. P. et al., 1993, Development, 118: 489-498

Yang, Z Y et al., 1995, Nature Medicine 1: 1052-1056

Young et al. 1998, Nat. Biotechnol. 16(10): 946-50

Zhu et al., 1993, Science 261: 209-212

Zoller et al. 1987, Nucl. Acids Res., 10: 6487

What is claimed is:

1. An isolated polypeptide comprising the sequence set forth in any one of SEQ ID NOs 2, 15 and 18, a biologically active fragment of any of said sequence having at least 6 amino acids in length, or a variant of any of said sequence having at least 85% sequence identity thereto, with the proviso that said fragment of SEQ ID NO: 2 comprises a contiguous sequence of amino acids contained within the sequence set forth in SEQ ID NO: 7.

2. An isolated polypeptide comprising the sequence set forth in SEQ ID NO: 18, or a biologically active fragment of said sequence having at least 6 amino acids in length, or a variant of said sequence having at least 85% sequence identity thereto.

3. The polypeptide of claim 2, wherein the biologically active fragment is at least 8 amino acid in length.

4. The polypeptide of claim 2, wherein the biologically active fragment is selected from the group consisting of residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 233-240, 241-248, 249-256, 257-264, 265-272, 273-280, 281-288, 289-296, 297-304, 305-312, 313-320, 321-328, 329-336, 337-344, 345-352, 353-360, 361-368, 369-376, 377-384, 385-392, 393-400, 401-408, 409-416, 417-424, 425-432, 433-440, 441-448, 449-456, 457-464, and 261-468 of SEQ ID NO: 18.

5. The polypeptide of claim 2, wherein the biologically active fragment comprises at least one domain selected from the group consisting of a SOX18 HMG box domain, SOX18 trans-activation domain, SOX18 conserved C terminal domain, and a portion of said domain having at least 6 amino acids in length.

6. The polypeptide of claim 5, wherein the conserved C terminal domain comprises the sequence set forth in SEQ ID NO: 30.

7. The polypeptide of claim 5, wherein the conserved C terminal domain comprises the sequence set forth in SEQ ID NO: 27.

8. The polypeptide of claim 5, wherein the portion comprises the sequence set forth in SEQ ID NO: 28 or 29.

9. The polypeptide of claim 5, wherein the HMG box domain comprises the sequence set forth in SEQ ID NO: 23.

10. The polypeptide of claim 5, wherein the trans-activation domain comprises the sequence set forth in SEQ ID NO: 25.

11. An isolated polypeptide comprising the sequence set forth in SEQ ID NO: 15, or a biologically active fragment of said sequence having at least 6 amino acids in length, or a variant of said sequence having at least 85% sequence identity thereto.

12. The polypeptide of claim 11, wherein the biologically active fragment is at least 8 amino acid in length.

13. The polypeptide of claim 12, wherein the biologically active fragment is selected from the group consisting of residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 233-240, 241-248, 249-256, 257-264, 265-272, 273-280, 281-288, 289-296, 297-304, 305-312, 313-320, 321-328, 329-336, and 333-340 of SEQ ID NO: 15.

14. The polypeptide of claim 11, wherein the biologically active fragment comprises at least one domain selected from the group consisting of a SOX18 HMG box domain, SOX18 trans-activation domain, SOX18 conserved C terminal domain, and a portion of said domain having at least 6 amino acids in length.

15. The polypeptide of claim 14, wherein the conserved C terminal domain comprises the sequence set forth in SEQ ID NO: 30.

16. The polypeptide of claim 14, wherein the conserved C terminal domain comprises the sequence set forth in SEQ ID NO: 27.

17. The polypeptide of claim 14, wherein the portion comprises the sequence set forth in SEQ ID NO: 28 or 29.

18. The polypeptide of claim 14, wherein the HMG box domain comprises the sequence set forth in SEQ ID NO: 23.

19. The polypeptide of claim 14, wherein the trans-activation domain comprises the sequence set forth in SEQ ID NO: 25.

20. An isolated polypeptide comprising the sequence set forth in any one of SEQ ID NO: 2, a biologically active fragment of a sequence set forth in SEQ ID NO: 2 having at least 6 amino acids in length, or a variant of a sequence set forth in said SEQ ID NO: 2 having at least 85% sequence identity thereto, with the proviso that said biologically active fragment of SEQ ID NO: 2 comprises a contiguous sequence of amino acids contained within the sequence set forth in SEQ ID NO: 7.

21. The polypeptide of claim 20, wherein the biologically active fragment is at least 8 amino acid in length.

22. The polypeptide of claim 21, wherein the biologically active fragment is selected from the group consisting of residues 1-8, 9-16, 17-24, 25-32, 33-40, 41-48, 49-56, 57-64, 65-72, 73-80, 81-88, and 84-91 of SEQ ID NO: 7.

23. The polypeptide of claim 20, wherein the biologically active fragment comprises at least one domain selected from the group consisting of a SOX18 HMG box domain, SOX18 trans-activation domain, SOX18 conserved C terminal domain, and a portion of said domain at least 6 amino acids in length.

24. The polypeptide of claim 23, wherein the conserved C terminal domain comprises the sequence set forth in SEQ ID NO: 30.

25. The polypeptide of claim 23, wherein the conserved C terminal domain comprises the sequence set forth in SEQ ID NO: 13.

26. The polypeptide of claim 23, wherein the portion comprises the sequence set forth in SEQ ID NO: 28 or 29.

27. The polypeptide of claim 23, wherein the HMG box domain comprises the sequence set forth in SEQ ID NO: 9.

28. The polypeptide of claim 23, wherein the trans-activation domain comprises the sequence set forth in SEQ ID NO: 11.

29. The polypeptide of claim 1, wherein said variant is distinguished from the sequence set forth in any one of SEQ ID NOs 2, 15 and 18, or biologically active fragments thereof, by the substitution of at least one amino acid.

30. The polypeptide of claim 29, wherein said substitution is a conservative substitution.

31. An isolated polynucleotide comprising a nucleotide sequence encoding a member selected from the group consisting of the amino acid sequences set forth in SEQ ID NO: 2, 15 or 18, a biologically active fragment of said amino acid sequence having at least 6 amino acids in length, a variant of said amino acid sequences having at least 85% sequence identity thereto, with the proviso that said fragment of SEQ ID NO: 2 comprises a contiguous sequence of amino acids contained within the sequence set forth in SEQ ID NO: 7.

32. The polynucleotide of claim 31, wherein the polynucleotide comprises the sequence set forth in any one of SEQ ID NOs: 1, 3, 5, 14, 16, 17, 19 and 21 ,or a fragment of any one of SEQ ID NOs: 1, 3, 5, 14, 16, 17, 19 and 21 thereof having at least 18 nucleotides in length, with the proviso that said fragment of any of SEQ ID NOs: 1, 3 or 5 comprises a contiguous sequence of nucleotides contained within the sequence set forth in SEQ ID NO.6.

33. The polynucleotide of claim 31, wherein the polynucleotide comprises a sequence that hybridizes under high stringency conditions to the sequence set forth in any one of SEQ ID NOs 1, 3, 5, 14, 16, 17, 19 and 21, or to a fragment f any one of SEQ ID NOs 1, 3, 5, 14, 16, 17, 19 and 21 thereof having at least 18 nucleotides in length.

34. An isolated polynucleotide comprising the sequence set forth in SEQ ID NO. 17, or a fragment thereof having at least 18 nucleotides in length.

35. An isolated polynucleotide which hybridizes under high stringency conditions to the sequence set forth in SEQ ID NO. 17, or a to fragment thereof having at least 18 nucleotides in length.

36. An isolated polynucleotide encoding a polypeptide which modulates an activity selected from the group consisting of cell differentiation, vasculogenesis, angiogenesis, hair follicle development, cell proliferation and tumorigenesis, wherein said polynucleotide hybridizes under high stringency conditions to the sequence set forth in SEQ ID NO: 17, or to a fragment thereof at least 18 nucleotides in length.

37. An isolated polynucleotide comprising the sequence set forth in SEQ ID NO: 19, or a fragment thereof having at least 18 nucleotides in length.

38. An isolated polynucleotide which hybridizes under high stringency conditions to the sequence set forth in SEQ ID NO. 19, or a to fragment thereof having at least 18 nucleotides in length.

39. An isolated polynucleotide encoding a polypeptide which modulates an activity selected from the group consisting of cell differentiation, vasculogenesis, angiogenesis, hair follicle development, cell proliferation and tumorigenesis, wherein said polynucleotide hybridizes under high stringency conditions to the sequence set forth in SEQ ID NO. 19, or to a fragment thereof having at least 18 nucleotides in length.

40. An isolated polynucleotide comprising the sequence set forth in SEQ ID NO. 21, or a fragment thereof having at least 18 nucleotides in length.

41. An isolated polynucleotide which hybridizes under high stringency conditions to the sequence set forth in SEQ ID NO. 21, or a to fragment thereof having at least 18 nucleotides in length.

42. An isolated polynucleotide encoding a polypeptide which modulates an activity selected from the group consisting of cell differentiation, vasculogenesis, angiogenesis, hair follicle development, cell proliferation and tumorigenesis, and which hybridizes under high stringency conditions to the sequence set forth in SEQ ID NO. 21, or to a fragment thereof having at least 18 nucleotides in length.

43. An isolated polynucleotide comprising the sequence set forth in SEQ ID NO. 1, or a fragment thereof having at least 18 nucleotides in length, with the proviso that said fragment comprises a contiguous sequence of nucleotides contained within the sequence set forth in any one of SEQ ID NOs 6, 38 and 39.

44. An isolated polynucleotide which hybridizes under high stringency conditions to the sequence set forth in SEQ ID NO.1, or a to fragment thereof having at least 18 nucleotides in length, with the proviso that said fragment comprises a contiguous sequence of nucleotides contained within the sequence set forth in any one of SEQ ID NO. 6, 38 and 39.

45. An isolated polynucleotide encoding a polypeptide which modulates an activity selected from the group consisting of cell differentiation, vasculogenesis, angiogenesis, hair follicle development, cell proliferation and tumorigenesis, and which hybridizes under high stringency conditions to the sequence set forth in SEQ ID NO: 1, or to a fragment thereof having at least 18 nucleotides in length, with the proviso that said fragment comprises a contiguous sequence of nucleotides contained within the sequence set forth in any one of SEQ ID NOs 6, 38 and 39.

46. An isolated polynucleotide comprising the sequence set forth in SEQ ID NO. 3, or a fragment thereof having at least 18 nucleotides in length, with the proviso that said fragment comprises a contiguous sequence of nucleotides contained within the sequence set forth in any one of SEQ ID NOs 6 and 38.

47. An isolated polynucleotide which hybridizes under high stringency conditions to the sequence set forth in SEQ ID NO. 3, or a to fragment thereof having at least 18 nucleotides in length, with the proviso that said fragment comprises a contiguous sequence of nucleotides contained within the sequence set forth in any one of SEQ ID NOs. 6 and 38.

48. An isolated polynucleotide encoding a polypeptide which modulates an activity selected from the group consisting of cell differentiation, vasculogenesis, angiogenesis, hair follicle development, cell proliferation and tumorigenesis, and which hybridizes under high stringency conditions to the sequence set forth in SEQ ID NO. 3, or to a fragment thereof having at least 18 nucleotides in length, with the proviso that said fragment comprises a contiguous sequence of nucleotides contained within the sequence set forth in any one of SEQ ID NOs 6 and 38.

49. An isolated polynucleotide comprising the sequence set forth in SEQ ID NO. 5, or a fragment thereof having at least 18 nucleotides in length, with the proviso that said fragment comprises a contiguous sequence of nucleotides contained within the sequence set forth in SEQ ID NO. 6.

50. An isolated polynucleotide which hybridizes under high stringency conditions to the sequence set forth in SEQ ID NO. 5, or a to fragment thereof at least 18 nucleotides in length, with the proviso that said fragment comprises a contiguous sequence of nucleotides contained within the sequence set forth in SEQ ID NO: 6.

51. An isolated polynucleotide encoding a polypeptide which modulates an activity selected from the group consisting of cell differentiation, vasculogenesis, angiogenesis, hair follicle development, cell proliferation and tumorigenesis, and which hybridizes under high stringency conditions to the sequence set forth in SEQ ID NO. 5, or to a fragment thereof having at least 18 nucleotides in length, with the proviso that said fragment comprises a contiguous sequence of nucleotides contained within the sequence set forth in SEQ ID NO. 6.

52. An isolated polynucleotide which hybridizes under high stringency conditions to the sequence set forth in SEQ ID NO. 5, or a fragment thereof having at least 24 nucleotides in length.

53. An isolated polynucleotide encoding a polypeptide which modulates an activity selected from the group consisting of cell differentiation, vasculogenesis, angiogenesis, hair follicle development, cell proliferation and tumorigenesis, and which hybridizes under high stringency conditions to the sequence set forth in SEQ ID NO. 5, or a fragment thereof having at least 24 nucleotides in length.

54. A vector including a polynucleotide comprising a nucleotide sequence encoding a member selected from the group consisting of the amino acid sequence set forth in SEQ ID NOs. 2, 15 or 18, a biologically active fragment of any of said sequence set forth in SEQ ID NOs. 2, 15 or 18 having at least 6 amino acids in length, a variant of said amino acid sequence having at least 85% sequence identity thereto, with the proviso that said fragment of SEQ ID NO: 2 comprises a contiguous sequence of amino acids contained within the sequence set forth in SEQ ID NO: 7.

55. A vector including a polynucleotide comprising the sequence set forth in any one of SEQ ID Nos. 1, 3, 5, 14, 16, 17, 19 and 21 or a fragment thereof having at least 18 nucleotides in length, with the proviso that said fragment of SEQ ID Nos. 1, 3 or 5 comprises a contiguous sequence of nucleotides contained within the sequence set forth in SEQ ID NO. 6.

56. An expression vector including a polynucleotide comprising a nucleotide sequence encoding a member selected from the group consisting of the amino acid sequence set forth in SEQ ID NOs. 2, 15 or 18, a biologically active fragment of said amino acid sequence having at least 6 amino acids in length, a variant of said amino acid sequence having at least 85% sequence identity thereto, with the proviso that said fragment of SEQ ID NO. 2 comprises a contiguous sequence of amino acids contained within the sequence set forth in SEQ ID NO. 7, wherein said polynucleotide is operably linked to a regulatory polynucleotide.

57. A vector including a polynucleotide comprising the sequence set forth in any one of SEQ ID Nos. 1, 3, 5, 14, 16, 17, 19 and 21 or a fragment thereof having at least 18 nucleotides in length, with the proviso that said fragment of SEQ ID NO. 1, 3 or 5 comprises a contiguous sequence of nucleotides contained within the sequence set forth in SEQ ID NO. 6, wherein said polynucleotide is operably linked to a regulatory polynucleotide.

58. A host cell containing the expression vector of claim 56 or claim 57.

59. A method of producing a recombinant polypeptide comprising the sequence set forth in any one of SEQ ID Nos. 2, 15 and 18, or a biologically active fragment of said sequence having at least 6 amino acids in length, or a variant of said sequence having at least 85% sequence identity thereto, with the proviso that said fragment of SEQ ID NO. 2 comprises a contiguous sequence of amino acids contained within the sequence set forth in SEQ ID NO. 7, said method comprising:
culturing a host cell containing the vector of claim 56 or claim 57 such that said recombinant polypeptide is expressed from said polynucleotide; and
isolating said recombinant polypeptide.

60. A method for modulating at least one activity selected from the group consisting of cell differentiation, vasculogenesis, angiogenesis, hair follicle development, cell proliferation and tumorigenesis, said method comprising introducing into a cell a member selected from the group consisting of the polypeptide sequence set forth in any one of SEQ ID Nos. 2, 15 and 18, a biologically active fragment of said sequence having at least 6 amino acids in length, or a variant of said sequence having at least 85% sequence identity thereto, with the proviso that said fragment of SEQ ID NO: 2 comprises a contiguous sequence of amino acids contained within the sequence set forth in SEQ ID NO. 7, and a polynucleotide from which said polypeptide, fragment, or variant can be translated.

61. The method of claim 60, comprising introducing into said cell a member selected from the group consisting of a polypeptide and a polynucleotide, wherein said polypeptide is selected from the group consisting of a SOX7 polypeptide, a SOX17 polypeptide, a biologically active fragment of said SOX7 polypeptide, a biologically active fragment of said SOX17 polypeptide, a variant of said SOX7 polypeptide, a variant of said SOX17 polypeptide, a variant of said SOX7 polypeptide fragment, a variant of said SOX17 polypeptide fragment, and wherein said polynucleotide comprises a nucleotide sequence from which said polypeptide can be translated.

62. The method of claim 61, wherein the SOX7 polypeptide comprises the sequence set forth in SEQ ID NO 34, or a variant having at least 70% sequence identity to SEQ ID NO. 34.

63. The method of claim 61, the SOX17 polypeptide comprises the sequence set forth in SEQ ID NO. 36, or a variant having at least 70% sequence identity to said SEQ ID NO. 36.

64. A method of screening for an agent which modulates at least one activity selected from the group consisting of cell differentiation, vasculogenesis, angiogenesis, hair follicle development, cell proliferation and tumorigenesis, cell proliferation and tumorigenesis, said method comprising:
contacting a preparation comprising a member selected from the group consisting of the polypeptide sequence set forth in any one of SEQ ID NOs. 2, 15 and 18, a biologically active fragment of said sequence having at least 6 amino acids in length, or a variant of said sequence having at least 85% sequence identity thereto, with the proviso that said fragment of SEQ ID NO. 2 comprises a contiguous sequence of amino acids contained within the sequence set forth in SEQ ID NO. 7, and a genetic sequence encoding said polypeptide, fragment or variant, with a test agent; and
detecting a change in the level and/or functional activity of said member or an expression product of said genetic sequence.

65. A method of screening for an agent which modulates at least one activity selected from the group consisting of cell differentiation, vasculogenesis, angiogenesis, hair follicle development, cell proliferation and tumorigenesis, said method comprising:
contacting a preparation comprising a member selected from the group consisting of a SOX7 polypeptide, a biologically active fragment of said polypeptide having at least 6 amino acids in length, or a variant of said polypeptide having at least 70% sequence identity thereto, and a genetic sequence encoding said polypeptide, fragment or variant, with a test agent; and
detecting a change in the level and/or functional activity of said member or an expression product of said genetic sequence.

66. The method of claim 65, wherein the SOX7 polypeptide comprises the sequence set forth in SEQ ID NO. 34.

67. A method of screening for an agent which modulates at least one activity selected from the group consisting of cell differentiation, vasculogenesis, angiogenesis, hair follicle development, cell proliferation and tumorigenesis, said method comprising:
contacting a preparation comprising a member selected from the group consisting of a SOX17 polypeptide, a biologically active fragment of said polypeptide having at least 6 amino acids in length, or a variant of said polypeptide having at least 70% sequence identity thereto, and a genetic sequence encoding said polypeptide, fragment or variant, with a test agent; and
detecting a change in the level and/or functional activity of said member or an expression product of said genetic sequence.

68. The method of claim 67, wherein the SOX7 polypeptide comprises the sequence set forth in SEQ ID NO. 36.

69. A method for detecting a specific polypeptide or polynucleotide sequence, comprising detecting a sequence of:
SEQ ID NO. 2, or a fragment thereof, having at least 6 amino acids residues in length comprising a contiguous sequence of amino acids contained within the sequence set forth in SEQ ID NO. 7;
SEQ ID NOs. 1, 3 or 5, or a fragment of SEQ ID NOs. 1, 3 or 5 thereof, having at least 18 nucleotides in length comprising a contiguous sequence of nucleotides contained within the sequence set forth in SEQ ID NO. 6;
SEQ ID NO. 18, or a fragment thereof having at least 6 amino acids residues in length;
SEQ ID NOs: 17, 19 or 21, or a fragment of SEQ ID Nos. 17, 19 or 21 thereof having at least 18 nucleotides in length;
SEQ ID NO. 15, or a fragment thereof having at least 6 amino acids residues in length; or
SEQ ID NOs 14, or 16, or a fragment of SEQ ID NOs 14, or 16 thereof having at least 18 nucleotides in length.

70. A method of detecting a member selected from the group consisting of the polypeptide sequence set forth in any one of SEQ ID Nos. 2, 15 and 18, a biologically active fragment of said sequence having at least 6 amino acids in length, or a variant of said sequence having at least 85% sequence identity thereto, with the proviso that said fragment of SEQ ID NO: 2 comprises a contiguous sequence of amino acids contained within the sequence set forth in SEQ ID NO. 7, said method comprising:
contacting a test polypeptide with a MEF2C polypeptide in a biological sample; and
detecting the presence of a complex comprising the MEF2C polypeptide and said test polypeptide in said biological sample.

71. An antigen-binding molecule that is specifically immuno-interactive with a member selected from the group consisting of a polypeptide comprising the sequence set forth in any one of SEQ ID Nos. 2, 15 and 18, and a biologically active fragment of said sequence having at least 6 amino acids in length, with the proviso that said biologically active fragment of SEQ ID NO: 2 comprises a contiguous sequence of amino acids contained within the sequence set forth in SEQ ID NO: 7.

72. A method of detecting in a biological sample a member selected from the group consisting of the polypeptide sequence set forth in any one of SEQ ID Nos. 2, 15 and 18; a biologically active fragment of said sequence having at least 6 amino acids in length, and a variant of said sequence having at least 85% sequence identity thereto, with the proviso that said fragment of SEQ ID NO: 2 comprises a contiguous sequence of amino acids contained within the sequence set forth in SEQ ID NO: 7, said method comprising:
contacting the sample with the antigen-binding molecule of claim 71; and
detecting the presence of a complex comprising said antigen-binding molecule and said member in said sample.

73. A method for detecting a polypeptide comprising the sequence set forth in any one of SEQ ID Nos. 2, 15 and 18, a biologically active fragment of said sequence having at least 6 amino acids in length, or a variant of said sequence having at least 85% sequence identity thereto, with the proviso that said fragment of SEQ ID NO. 2 comprises a contiguous sequence of amino acids contained within the sequence set forth in SEQ ID NO. 7, said method comprising
detecting expression in a cell of a polynucleotide encoding said polypeptide.

74. A method for detecting an activity selected from the group consisting of cell differentiation, vasculogenesis, angiogenesis and hair follicle development, comprising:
detecting in a biological sample an expression product from at least one subgroup F SOX polynucleotide.

75. The method of claim 74, wherein the subgroup F SOX polynucleotide encodes a polypeptide selected from the group consisting of SOX7, SOX17 and SOX18.

76. An agent which modulates at least one activity selected from the group consisting of cell differentiation, vasculogenesis, angiogenesis, hair follicle development, cell proliferation and tumorigenesis, wherein said agent is obtained or identified by a method comprising:
contacting a preparation comprising a member selected from the group consisting of a subgroup F SOX polypeptide, a biologically active fragment of said polypeptide having at least 6 amino acids in length, a variant of said polypeptide having at least 85% sequence identity thereto, and a genetic sequence encoding said polypeptide, fragment or variant, with a test agent; and
detecting a change in the level and/or functional activity of said member or an expression product of said genetic sequence.

77. The agent of claim 76, wherein the subgroup F SOX polypeptide is selected from the group consisting of SOX7, SOX17 and SOX18.

78. A method for modulating at least one activity selected from the group consisting of cell differentiation, vasculogenesis, angiogenesis, hair follicle development, cell proliferation and tumorigenesis, said method comprising introducing into said cell the agent of claim 76 for a time and under conditions sufficient to modulate the level and/or functional activity of said SOX polypeptide.

79. The method of claim 78, wherein the agent increases the level and/or functional activity of said SOX polypeptide.

80. The method of claim 79, wherein the activity that is modulated is selected from the group consisting of cell differentiation, vasculogenesis, angiogenesis, and hair follicle development.

81. The method of claim 78, wherein the agent decreases the level and/or functional activity of said SOX polypeptide.

82. The method of claim 81, wherein the activity that is modulated is preferably cell proliferation or tumorigenesis.

83. A composition for treatment and/or prophylaxis of at least one condition selected from the group consisting of artherosclerosis, cancer, restenosis, pulmonary disease, tissue injury and hair loss, comprising at least one member selected from the group consisting of a polypeptide comprising a subgroup F SOX polypeptide, a biologically active fragment of said polypeptide, a variant of said polypeptide, a variant of said fragment, a polynucleotide comprising a nucleotide sequence from which said polypeptide can be translated, and an agent that enhances the level and/or functional activity of said polypeptide, together with a pharmaceutically acceptable carrier, wherein said agent has been obtained or identified by a method comprising:
contacting a preparation comprising a member selected from the group consisting of a subgroup F SOX polypeptide, a biologically active fragment of said polypeptide having at least 6 amino acids in length, or a variant of said polypeptide having at least 85% sequence identity thereto, and a genetic sequence encoding said polypeptide, fragment or variant, with a test agent; and
detecting an increase in the level and/or functional activity of said member or an expression product of said genetic sequence.

84. The composition of claim 83, wherein the subgroup F SOX polypeptide is selected from the group consisting of SOX7, SOX17 and SOX18.

85. A composition for treatment and/or prophylaxis of tumorigenesis, comprising an agent that reduces the level and/or functional activity of at least one subgroup F SOX polypeptide, together with a pharmaceutically acceptable carrier, wherein said agent has been obtained or identified by a method comprising:
contacting a preparation comprising a member selected from the group consisting of a subgroup F SOX polypeptide, a biologically active fragment of said polypeptide at least 6 amino acids in length, or a variant of said polypeptide having at least 85% sequence identity thereto, and a genetic sequence encoding said polypeptide, fragment or variant, with a test agent; and
detecting a reduction in the level and/or functional activity of said member or an expression product of said genetic sequence.

86. The composition of claim 85, wherein the subgroup F SOX polypeptide is selected from the group consisting of SOX7, SOX17 and SOX18.

87. A composition comprising one or more agents that enhances the level and/or functional activity of at least two subgroup F SOX polypeptides, wherein said one or more agents are obtained or identified by a method comprising:
contacting a preparation comprising a member selected from the group consisting of a subgroup F SOX polypeptide, a biologically active fragment of said polypeptide at least 6 amino acids in length, or a variant of said polypeptide having at least 85% sequence identity thereto, and a genetic sequence encoding said polypeptide, fragment or variant, with a test agent; and
detecting an increase in the level and/or functional activity of said member or an expression product of said genetic sequence.

88. The composition of claim 87, wherein the subgroup F SOX polypeptide is selected from the group consisting of SOX7, SOX17 and SOX18.

89. A composition comprising one or more agents that enhances the level and/or functional activity of all subgroup F SOX polypeptides, wherein the or each agent has been obtained or identified by a method comprising:
contacting a preparation comprising a member selected from the group consisting of a subgroup F SOX polypeptide, a biologically active fragment of said polypeptide at least 6 amino acids in length, or a variant of said polypeptide having at least 85% sequence identity thereto, and a genetic sequence encoding said polypeptide, fragment or variant, with a test agent; and
detecting an increase in the level and/or functional activity of said member or an expression product of said genetic sequence.

90. The composition of claim 89, wherein the subgroup F SOX polypeptide is selected from the group consisting of SOX7, SOX17 and SOX18.

91. A method for treatment and/or prophylaxis of at least one condition selected from the group consisting of artherosclerosis, cancer, restenosis, pulmonary disease, tissue injury and hair loss, said method comprising administering to a patient in need of such treatment a therapeutically effective amount of at least one member selected from the group consisting of a polypeptide selected from the group consisting of a subgroup F SOX polypeptide, a biologically active fragment of said polypeptide, a variant of said polypeptide, a variant of said fragment, a polynucleotide comprising a nucleotide sequence from which said polypeptide can be translated, and an agent that enhances the level and/or functional activity of said polypeptide, and optionally together with a pharmaceutically acceptable carrier, wherein said agent has been obtained or identified by a method comprising:
contacting a preparation comprising a member selected from the group consisting of a subgroup F SOX polypeptide, a biologically active fragment of said polypeptide at least 6 amino acids in length, or a variant of said polypeptide having at least 85% sequence identity thereto, and a genetic sequence encoding said polypeptide, fragment or variant, with a test agent; and
detecting an increase in the level and/or functional activity of said member or an expression product of said genetic sequence.

92. A method for treatment and/or prophylaxis of tumorigenesis, comprising administering to a patient in need of such treatment a therapeutically effective amount of one or more agents that reduce the level and/or functional activity of at least one subgroup F SOX polypeptide, and optionally together with a pharmaceutically acceptable carrier, wherein said agent has been obtained or identified by a method comprising:
contacting a preparation comprising a member selected from the group consisting of a subgroup F SOX polypeptide, a biologically active fragment of said polypeptide having at least 6 amino acids in length, or a variant of said polypeptide having at least 85% sequence identity thereto, and a genetic sequence encoding said polypeptide, fragment or variant, with a test agent; and
detecting a reduction in the level and/or functional activity of said member or an expression product of said genetic sequence.

93. A method for modulating an activity selected from the group consisting of cell differentiation, vasculogenesis, angiogenesis, hair follicle development, cell proliferation and tumorigenesis, comprising:
contacting a cell with an agent, which modulates the level and/or functional activity of a subgroup F SOX polypeptide, for a time and under conditions sufficient to modulate said activity.

94. A method for promoting, augmenting or otherwise enhancing cell differentiation, comprising:
contacting a cell with an agent, which increases the level and/or functional activity of at least one subgroup F SOX polypeptide, for a time and under conditions sufficient to promote, augment or otherwise enhance cell differentiation.

95. A method for delaying, repressing or otherwise inhibiting cell proliferation or tumongenesis, comprising:
contacting a cell with an agent, which increases the level and/or functional activity of at least one subgroup F SOX polypeptide, for a time and under conditions sufficient to delay, repress or otherwise inhibit cell proliferation.
